(19) United States
(12) Patent Application Publication
(10) Pub. No.: US 2026/0103494 A1
(43) Pub. Date: Apr. 16, 2026

(54) GENE THERAPY TREATMENT

(71) Applicant: UNIVERSITY OF SHEFFIELD, Sheffield (GB)

(72) Inventors: Mimoun AZZOUZ, Sheffield (GB); Christopher WEBSTER, Sheffield (GB)

(73) Assignee: UNIVERSITY OF SHEFFIELD, Sheffield (GB)

(21) Appl. No.: 19/116,588

(22) PCT Filed: Oct. 12, 2023
(86) PCT No.: PCT/EP2023/078427

§ 371 Date: Mar. 28, 2025

(30) Foreign Application Priority Data
Oct. 14, 2022 (GB) 2215198.9

(51) Int. Cl. C07K 14/47; A61K 38/17; A61K 48/00; A61P 25/28; C12N 15/86; G01N 33/68

(52) U.S. Cl. CPC C07K 14/47; A61K 38/1709; A61K 48/0058; A61P 25/28; C12N 15/86; G01N 33/6896; C12N 2750/14143; C12N 2830/008; C12N 2830/50; G01N 2800/28; G01N 2800/52

(57) ABSTRACT

This disclosure concerns transcription cassettes comprising nucleic acid molecules comprising a nucleotide sequence encoding AP-4 subunits; vectors comprising said transcription cassettes; pharmaceutical compositions comprising said vector; and vectors or compositions for use in the treatment of AP-4-Hereditary Spastic Paraplegia.

### FIELD OF THE DISCLOSURE

This disclosure concerns transcription cassettes comprising nucleic acid molecules comprising a nucleotide sequence encoding at least one subunit of the heterotetrametric adaptor protein complex 4 (AP-4); vectors comprising said transcription cassettes; pharmaceutical compositions comprising said vector; and vectors or compositions for use in the treatment of AP-4 hereditary spastic paraplegias.

### BACKGROUND TO THE DISCLOSURE

Hereditary Spastic Paraplegias (HSPs) are a family of rare inherited, progressive, lower-limb spasticity disorders with an overall prevalence of 0.5-5.5 individuals per 100,000. Hereditary spastic paraplegia (HSP) in young patients is often characterised by weakness and spasticity (stiffness) of the legs and can later in life lead to further complications and may require the assistance of a cane, walker or wheelchair. There are a variety of genetic types of HSP such as autosomal dominant, autosomal recessive, X-linked, and maternally inherited (mitochondrial) forms with the autosomal dominant form most commonly found affecting between 75-80% of HSP patients. A variety of diagnostic methods for the identification of mutations in genes responsible for different forms of HSP, such as autosomal-recessive HSP (AR-HSP) caused by mutations in genes KIAA1840 (U.S. Pat. No. 10,519,503) or ZFYVE26 (US2017152562), or autosomal-dominant HSP caused by mutations in SPG3A, are disclosed in CN1958605.

AP-4-associated hereditary spastic paraplegia (AP-4-HSP), sometimes known as AP-4 deficiency syndrome or Adaptor protein complex 4 (AP-4) deficiency, is caused by loss-of-function mutations in any one of the four genes encoding the protein subunits of the AP-4 adaptor complex [10]. AP-4-HSP is autosomal recessive in nature. AP-4-HSP that is caused by mutations in the AP4B1 gene is sometimes called spastic paraplegia type 47 (SPG47) or hereditary spastic paraplegia 47 (HSP47) and it results in a significant decrease in AP4B1 protein levels [2]. AP-4-HSP may also be caused by mutations in the three other AP-4 subunits: AP4M1 mutations cause AP-4-HSP which is sometimes called SPG50 or HSP50, AP4E1 mutations cause AP-4-HSP which is sometimes called SPG51 or HSP51 and AP4S1 mutations cause AP-4-HSP which is sometimes called SPG52 or HSP51. AP-4-HSP characteristics are very similar regardless of the gene in which the causative mutations occur. The onset of AP-4-HSP usually occurs in early childhood and results in spasticity, intellectual disability from moderate to severe, impaired or absent speech, microencephaly, seizures, a shy character and in severe cases tetraplegia [11]. AP-4-HSP has so far been characterised in 199 children worldwide [1], however, incidents are most likely underreported. AP-4-HSP is progressive and there are no disease-modifying treatments. There is therefore a need to develop new therapies to improve patient outcomes for those suffering from AP-4-HSP.

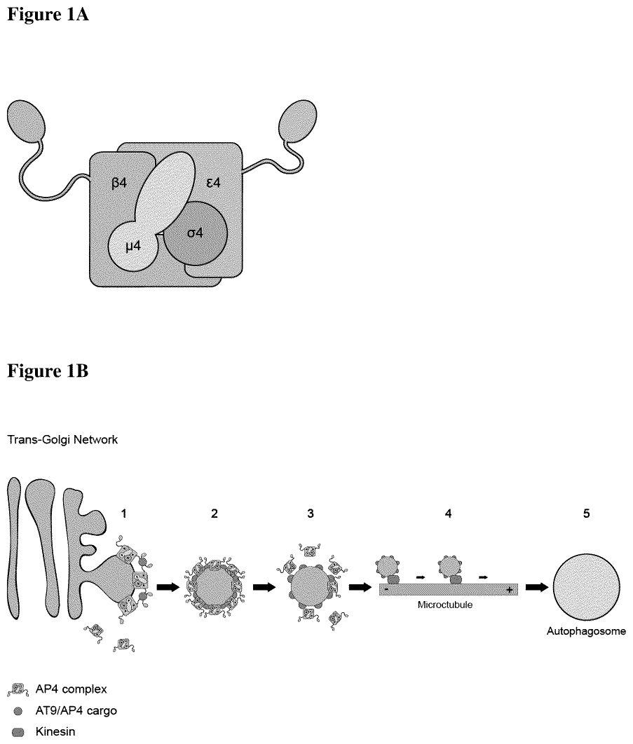

AP4B1 is one component of the AP-4 heterotetramer (FIG. 1A). The complete AP-4 complex is composed of two large adaptins (epsilon-type subunit AP4E1 and the beta-type subunit AP4B1), a medium adaptin (mu-type subunit AP4M1) and a small adaptin (sigma-type AP4S1). The AP-4 complex forms a non clathrin-associated coat on vesicles departing the trans-Golgi network (TGN) and may be involved in the targeting of proteins from the trans-Golgi network to the endosomal-lysosomal system (FIG. 1B). It is also involved in protein sorting to the basolateral membrane in epithelial cells and the proper asymmetric localization of proteins in neurons. AP-4 positive TGN derived vesicles are essential for correct spatial formation of autophagosomes and loss of the AP-4 complex can therefore impair autophagosome formation in the distal axon. Thus, the AP-4 complex is key to normal functioning in the brain.

Adeno-associated virus (AAV) vectors are known in the art and offer, when compared to retroviral or lentiviral vectors, a variety of advantages such as their mild immune response, capability to infect a broad range of cells and that the desired DNA is not integrated into the genome resulting in potential disruption and knock out of other genes but is stored extrachromosomal in the cell. AAV comprises single-stranded DNA genome of approximately 4.8 kilobases (kb) comprising three genes with coding sequences flanked by inverted repeats which are required for genome replication and packaging. Uses of AAVs and modified AAV vectors are known in the art and disclosed in WO2019/032898, WO2020041498 or WO2019/028306. AAV vectors have completed a variety of phase I and II clinical trials for the delivery of genes in the treatment of cystic fibroses and congestive heart failure and approved therapies for the treatment of spinal muscular atrophy.

In our co-pending application WO2021/205028, the content of which is incorporated in its entirety, we disclose transcription cassettes comprising nucleic acid molecules encoding AP-4 polypeptides.

We herein disclose optimised expression vectors that include AP-4 nucleic acid molecules operably linked to expression control sequences adapted for expression in mammalian neurones, for example motor neurones, and the use of the modified expression vectors to deliver and functionally replace dysfunctional AP-4 proteins in the prevention or treatment of symptoms associated with HSPs. This disclosure relates to the development of modified vectors, for example AAV vectors, enhanced AAV vectors, including nucleic acid molecules encoding proteins of the AP-4 complex.

### STATEMENTS OF INVENTION

According to an aspect of the invention there is provided an isolated nucleic acid molecule comprising: a transcription cassette comprising in a 5′ to 3′ direction between first and second inverted repeat sequences:
i) a promoter adapted for expression in a mammalian neurone wherein said promoter is associated with an enhancer nucleotide motif;
ii) an intron nucleotide sequence; and
iii) a polyadenylation signal nucleotide sequence; wherein said cassette further comprises a nucleic acid molecule comprising a nucleotide sequence that encodes at least one protein of the AP-4 complex.

In a preferred embodiment of the invention said enhancer motif is a CMV enhancer.

In a preferred embodiment of the invention said CMV enhancer motif comprises or consists of the nucleotide sequence in SEQ ID NO: 1, or polymorphic nucleotide sequence variant thereof,

Preferably said hybrid intron comprises or consists of the nucleotide sequence in SEQ ID NO: 2, or polymorphic sequence variant thereof.

In a preferred embodiment of the invention said polyadenylation signal is a growth hormone (GH) polyadenylation signal.

Preferably, said GH polyadenylation signal comprises or consists of the nucleotide sequence in SEQ ID NO: 4, or polymorphic sequence variant thereof.

In a preferred embodiment of the invention said promoter is the chicken beta actin promoter.

Preferably, said chicken beta actin promoter comprises or consists of the nucleotide sequence in SEQ ID NO: 3.

Preferably, said chicken beta actin promoter comprises or consists of the nucleotide sequence in SEQ ID NO: 28.

In a preferred embodiment of the invention said transcription cassette comprises or consists of the nucleotide sequence in SEQ ID NO: 9.

A polymorphic sequence variant is a sequence that varies from a reference sequence by one or more nucleotides bases, for example, 2, 3, 4, 5 or more bases.

In a preferred embodiment of the invention said expression cassette comprises a nucleic acid molecule comprising a nucleotide sequence selected from the group consisting of:
i) a nucleotide sequence, or polymorphic sequence variant, as set forth in SEQ ID NO: 15 (AP4B1);
ii) a nucleotide sequence wherein said sequence is degenerate as a result of the genetic code to the nucleotide sequence defined in (i);
iii) a nucleic acid molecule the complementary strand of which hybridizes under stringent hybridization conditions to the sequence in SEQ ID NO: 15 (AP4B1) wherein said nucleic acid molecule encodes a polypeptide that forms a complex with polypeptides comprising the AP-4 complex;
iv) a nucleotide sequence that encodes a polypeptide comprising an amino acid sequence as represented in SEQ ID NO: 16 (AP4B1);
v) a nucleotide sequence that encodes a polypeptide comprising an amino acid sequence wherein said amino acid sequence is modified by addition deletion or substitution of at least one amino acid residue as represented in iv) wherein said polypeptide forms a complex with polypeptides comprising the AP-4 complex.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, NY, 2001); and Tijssen, Laboratory Techniques in Biochemistry and Molecular Biology-Hybridization with Nucleic Acid Probes Part I, Chapter 2 (Elsevier, New York, 1993). The Tm is the temperature at which 50% of a given strand of a nucleic acid molecule is hybridized to its complementary strand. The following is an exemplary set of hybridization conditions and is not limiting:

Very High Stringency (allows sequences that share at least 90%, 91%, 92%, 93%, 94%, 95%, 96%, 97%, 98% or 99% identity to hybridize)
Hybridization: 5×SSC at 65° C. for 16 hours
Wash twice: 2×SSC at room temperature (RT) for 15 minutes each
Wash twice: 0.5×SSC at 65° C. for 20 minutes each

High Stringency (allows sequences that share at least 80%, 81%, 82%, 83%, 84%, 85%, 86%, 87%, 88%, or 89% identity to hybridize)
Hybridization: 5×-6×SSC at 65° C.-70° C. for 16-20 hours
Wash twice: 2×SSC at RT for 5-20 minutes each
Wash twice: 1×SSC at 55° C.-70° C. for 30 minutes each

Low Stringency (allows sequences that share at least 50%, 55%, 60%, 65%, 70% or 75% identity to hybridize)
Hybridization: 6×SSC at RT to 55° C. for 16-20 hours
Wash at least twice: 2×-3×SSC at RT to 55° C. for 20-30 minutes each.

In a preferred embodiment of the invention said expression cassette comprises a nucleic acid molecule comprising a nucleotide sequence selected from the group consisting of:
i) a nucleotide sequence, or polymorphic sequence variant, as set forth in SEQ ID NO: 17 (AP4E1);
a nucleotide sequence wherein said sequence is degenerate as a result of the genetic code to the nucleotide sequence defined in (i);
iii) a nucleic acid molecule the complementary strand of which hybridizes under stringent hybridization conditions to the sequence in SEQ ID NO: 17 (AP4E1);
wherein said nucleic acid molecule encodes a polypeptide that forms a complex with polypeptides comprising the AP-4 complex;
iv) a nucleotide sequence that encodes a polypeptide comprising an amino acid sequence as represented in SEQ ID NO: 18 (AP4E1);
v) a nucleotide sequence that encodes a polypeptide comprising an amino acid sequence wherein said amino acid sequence is modified by addition deletion or substitution of at least one amino acid residue as represented in iv) wherein said polypeptide forms a complex with polypeptides comprising the AP-4 complex.

In a preferred embodiment of the invention said expression cassette comprises a nucleic acid molecule comprising a nucleotide sequence selected from the group consisting of:
i) a nucleotide sequence, or polymorphic sequence variant, as set forth in SEQ ID NO: 19 (AP4M1);
ii) a nucleotide sequence wherein said sequence is degenerate as a result of the genetic code to the nucleotide sequence defined in (i);
iii) a nucleic acid molecule the complementary strand of which hybridizes under stringent hybridization conditions to the sequence in SEQ ID NO: 19 (AP4M1);
wherein said nucleic acid molecule encodes a polypeptide that forms a complex with polypeptides comprising the AP-4 complex;
iv) a nucleotide sequence that encodes a polypeptide comprising an amino acid sequence as represented in SEQ ID NO: 20 (AP4M1);
v) a nucleotide sequence that encodes a polypeptide comprising an amino acid sequence wherein said amino acid sequence is modified by addition deletion or substitution of at least one amino acid residue as represented in iv) wherein said polypeptide forms a complex with polypeptides comprising the AP-4 complex.

In a preferred embodiment of the invention said expression cassette comprises a nucleic acid molecule comprising a nucleotide sequence selected from the group consisting of:
i) a nucleotide sequence, or polymorphic sequence variant, as set forth in SEQ ID NO: 21 (AP4S1);
ii) a nucleotide sequence wherein said sequence is degenerate as a result of the genetic code to the nucleotide sequence defined in (i);
iii) a nucleic acid molecule the complementary strand of which hybridizes under stringent hybridization conditions to the sequence in SEQ ID NO: 21 (AP4S1);
wherein said nucleic acid molecule encodes a polypeptide that forms a complex with polypeptides comprising the AP-4 complex;
iv) a nucleotide sequence that encodes a polypeptide comprising an amino acid sequence as represented in SEQ ID NO: 22 (AP4S1);
a nucleotide sequence that encodes a polypeptide comprising an amino acid sequence wherein said amino acid sequence is modified by addition deletion or substitution of at least one amino acid residue as represented in iv) wherein said polypeptide forms a complex with polypeptides comprising the AP-4 complex.

In a preferred embodiment of the invention said cassette is adapted for expression in a motor neurone.

In a preferred embodiment of the invention said nucleic acid molecule comprises or consists of a nucleotide sequence as represented in SEQ ID NO: 15, or polymorphic sequence variant thereof.

In a preferred embodiment of the invention there is provided a nucleotide sequence or polymorphic sequence variant thereof, that encodes a polypeptide comprising an amino acid sequence as represented in SEQ ID NO: 16.

In a preferred embodiment of the invention said nucleic acid molecule comprises or consists of a nucleotide sequence as represented in SEQ ID NO: 17, or polymorphic sequence variant thereof.

In a preferred embodiment of the invention there is provided a nucleotide sequence or polymorphic sequence variant thereof that encodes a polypeptide comprising an amino acid sequence as represented in SEQ ID NO: 18.

In a preferred embodiment of the invention said nucleic acid molecule comprises or consists of a nucleotide sequence as represented in SEQ ID NO: 19, or polymorphic sequence variant thereof.

In a preferred embodiment of the invention there is provided a nucleotide sequence or polymorphic sequence variant thereof that encodes a polypeptide comprising an amino acid sequence as represented in SEQ ID NO: 20.

In a preferred embodiment of the invention said nucleic acid molecule comprises or consists of a nucleotide sequence as represented in SEQ ID NO: 21, or polymorphic sequence variant thereof.

In a preferred embodiment of the invention there is provided a nucleotide sequence or polymorphic sequence variant thereof, that encodes a polypeptide comprising an amino acid sequence as represented in SEQ ID NO: 22.

A polypeptide as herein disclosed may differ in amino acid sequence by one or more substitutions, additions, deletions, truncations that may be present in any combination. Among preferred variants are those that vary from a reference polypeptide by conservative amino acid substitutions. Such substitutions are those that substitute a given amino acid by another amino acid of like characteristics. The following non-limiting list of amino acids are considered conservative replacements (similar): a) alanine, serine, and threonine; b) glutamic acid and aspartic acid; c) asparagine and glutamine d) arginine and lysine; e) isoleucine, leucine, methionine and valine and f) phenylalanine, tyrosine and tryptophan. Most highly preferred are variants that retain or enhance the same biological function and activity as the reference polypeptide from which it varies. In one embodiment, the polypeptides have at least 70% identity, even more preferably at least 75% identity, still more preferably at least 80%, 85%, 90%, 95% identity, and at least 99% identity the full-length amino acid sequence or nucleotide sequence illustrated herein.

In an alternative preferred embodiment of the invention said promoter is a constitutive promoter.

In a further alternative embodiment of the invention said promoter is a regulated promoter, for example an inducible or cell specific promoter.

In a preferred embodiment of the invention said promoter is selected from the group consisting of: chicken beta actin (CBA) promoter, chicken beta actin hybrid (CBh) promoter, CAG promoter, JeT promoter, neuronal and glial specific promoters including synapsin 1, Hb9, MeP229 and GFAP promoter sequences, as well as AP-4 subunit specific promoter regions including AP4B1, AP4E1, AP4M1 and AP4S1.

In a preferred embodiment of the invention said promoter is chicken beta actin hybrid (CBh) promoter as set forth in SEQ ID NO: 9.

In an alternative preferred embodiment of the invention said promoter is chicken beta actin hybrid (CBh) promoter as set forth in SEQ ID NO: 28.

In an alternative preferred embodiment of the invention said promoter is the JeT promoter comprising or consisting of the nucleotide sequence in SEQ ID NO: 5.

In an alternative preferred embodiment of the invention said promoter is the hSyn promoter comprising or consisting of the nucleotide sequence in SEQ ID NO: 6.

In an alternative preferred embodiment of the invention said promoter is the MeP229 promoter comprising or consisting of the nucleotide sequence in SEQ ID NO: 7.

In an alternative preferred embodiment of the invention said promoter is the AP4B1 promoter comprising or consisting of the nucleotide sequence in SEQ ID NO: 8.

“Promoter” or “transcription promoter” are art recognised and, for the sake of clarity, includes the following features which are provided by example only, and not by way of limitation. Enhancer elements are cis acting nucleic acid sequences often found 5′ to the transcription initiation site of a gene (enhancers can also be found 3′ to a gene sequence or even located in intronic sequences). Enhancers, for example CMV enhancers, function to increase the rate of transcription of the gene to which the enhancer is linked. Enhancer activity is responsive to trans acting transcription factors (polypeptides) which have been shown to bind specifically to enhancer elements. The binding/activity of transcription factors (please see Eukaryotic Transcription Factors, by David S Latchman, Academic Press Ltd, San Diego) is responsive to several physiological/environmental cues and can be constitutive or regulatable and also cell/tissue specific. Promoter elements also include so called TATA box and RNA polymerase initiation selection (RIS) sequences which function to select a site of transcription initiation. These sequences also bind polypeptides which function, inter alia, to facilitate transcription initiation selection by RNA polymerase.

As used herein, first nucleic acid comprising a promoter sequence and second nucleotide sequence encoding a polypeptide are said to be “operably” linked when they are covalently linked in such a way as to place the expression or transcription of the second nucleic acid molecule under the control of the first nucleic acid molecule comprising regulatory sequences. If it is desired that the coding sequences be translated into a functional protein, two DNA sequences are said to be operably linked if induction of a promoter in the 5′ regulatory sequences result in the transcription of the coding sequence and production of mRNA. Thus, a promoter region would be operably linked to a coding sequence if the promoter region were capable of effecting transcription of that DNA sequence such that the resulting transcript IS translated into the desired protein or polypeptide.

According to a further aspect of the invention there is provided an expression vector comprising a transcription cassette according to the invention.

Viruses are commonly used as vectors for the delivery of exogenous genes. Commonly employed vectors include recombinantly modified enveloped or non-enveloped DNA and RNA viruses, for example baculoviridiae, parvoviridiae, picornoviridiae, herpesveridiae, poxviridae, adenoviridiae, picornaviridae or retroviridae e.g., lentivirus. Chimeric vectors may also be employed which exploit advantageous elements of each of the parent vector properties (See e.g., Feng, et al (1997) Nature Biotechnology 15:866-870). Such viral vectors may be wild-type or may be modified by recombinant DNA techniques to be replication deficient, conditionally replicating or replication competent. Conditionally replicating viral vectors are used to achieve selective expression in particular cell types while avoiding untoward broad-spectrum infection. Examples of conditionally replicating vectors are described in Pennisi, E. (1996) Science 274:342-343; Russell, and S. J. (1994) Eur. J. of Cancer 30A (8): 1165-1171.

Preferred vectors are derived from the adenoviral, adeno-associated viral or retroviral genomes.

In a preferred embodiment of the invention said expression vector is a viral based expression vector.

In a preferred embodiment of the invention said viral based vector is an adeno-associated virus [AAV].

In a preferred embodiment said viral based vector is selected from the group consisting of: AAV2, AAV3, AAV6, AAV13; AAV1, AAV4, AAV5, AAV6, AAV9 and rhAAV10.

In a preferred embodiment of the invention said viral based vector is AAV9.

In a preferred embodiment of the invention said viral based vector is an enhanced AAV9 vector, for example a PHP-b vector.

In a preferred embodiment of the invention said AAV vector is based on a single stranded AAV virus.

In an alternative embodiment of the invention said AAV vector is based on a self-complementary AAV virus.

Naturally occurring AAV serotypes typically comprise a single stranded genome which during natural infection is replicated to form a double stranded AAV viral genome. This is a rate limiting step in AAV replication and expression. A recombinant form of AAV is referred to as self-complementary AAV which comprise both a sense and antisense genomic strands that are adapted for immediate expression and replication. The viral based vector can comprise the gene encoding kanamycin resistance or for therapy can lack the gene encoding kanamycin resistance.

In a preferred embodiment of the invention said viral based vector comprises the nucleotide sequence set forth in SEQ ID NO: 10.

In a preferred embodiment of the invention said viral based vector comprises the nucleotide sequence set forth in SEQ ID NO: 11.

In a preferred embodiment of the invention said viral based vector comprises the nucleotide sequence set forth in SEQ ID NO: 12.

In a preferred embodiment of the invention said viral based vector comprises the nucleotide sequence set forth in SEQ ID NO: 13.

In a preferred embodiment of the invention said viral based vector comprises the nucleotide sequence set forth in SEQ ID NO: 14.

In a preferred embodiment of the invention said viral based vector comprises the nucleotide sequence set forth in SEQ ID NO: 23.

In a preferred embodiment of the invention said viral based vector comprises the nucleotide sequence set forth in SEQ ID NO: 24.

In a preferred embodiment of the invention said viral based vector comprises the nucleotide sequence set forth in SEQ ID NO: 25.

In a preferred embodiment of the invention said viral based vector comprises the nucleotide sequence set forth in SEQ ID NO: 26.

In a preferred embodiment of the invention said viral based vector comprises the nucleotide sequence set forth in SEQ ID NO: 27.

Preferably, said viral based vector sequence selected from the group consisting of SEQ ID NO 10-14 and 23-27 lacks the kanamycin resistance gene and is flanked by 5′ and 3′ Inverted Terminal Repeat (ITR) sequences. As known in the prior art, only the sequences between the ITRs are packaged into the clinical viral vector and delivered to patients. The ITR flanked transgene encoded within the recombinant AAV persists subsequently as an episome in the nucleus of the transduced cell such as non-dividing neuronal cells and providing long term expression.

In an alternative preferred embodiment of the invention said viral based vector is a lentiviral vector.

According to a further aspect of the invention there is provided a pharmaceutical composition comprising an expression vector according to the invention and an excipient or carrier.

The expression vector compositions of the present invention are administered in pharmaceutically acceptable preparations. Such preparations may routinely contain pharmaceutically acceptable concentrations of salt, buffering agents, preservatives, compatible carriers and supplementary therapeutic agents. The expression vector compositions of the invention can be administered by any conventional route, including injection or by gradual infusion over time.

The expression vector compositions of the invention are administered in effective amounts. An “effective amount” is that amount of the expression vector that alone, or together with further doses, produces the desired response. In the case of treating a disease, the desired response is inhibiting the progression of the disease. This may involve only slowing the progression of the disease temporarily, although more preferably, it involves halting the progression of the disease permanently. This can be monitored by routine methods. Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner.

These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The expression vector compositions used in the foregoing methods preferably are sterile and contain an effective amount of expression vector according to the invention for producing the desired response in a unit of weight or volume suitable for administration to a patient. The doses of vector administered to a subject can be chosen in accordance with different parameters, in particular in accordance with the mode of administration used and the state of the subject. Other factors include the desired period of treatment. If a response in a subject is insufficient at the initial doses applied, higher doses (or effectively higher doses by a different, more localized delivery route) may be employed to the extent that patient tolerance permits. Other protocols for the administration of vector compositions will be known to one of ordinary skill in the art, in which the dose amount, schedule of injections, sites of injections, mode of administration and the like vary from the foregoing. The administration of compositions to mammals other than humans, (e.g., for testing purposes or veterinary therapeutic purposes), is carried out under substantially the same conditions as described above. A subject, as used herein, is a mammal, preferably a human, and including a non-human primate, cow, horse, pig, sheep, goat, dog, cat or rodent.

When administered, the expression vector compositions of the invention are applied in pharmaceutically acceptable amounts and in pharmaceutically acceptable compositions. The term “pharmaceutically acceptable” means a non-toxic material that does not interfere with the effectiveness of the biological activity of the active agent. Such preparations may routinely contain salts, buffering agents, preservatives, compatible carriers, and optionally other therapeutic agents' (e.g., those typically used in the treatment of the specific disease indication). When used in medicine, the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically acceptable salts can be prepared as alkaline metal or alkaline earth salts, such as sodium, potassium or calcium salts.

The pharmaceutical compositions containing the expression vectors according to the invention may contain suitable buffering agents, including acetic acid in a salt; citric acid in a salt; boric acid in a salt; and phosphoric acid in a salt. The pharmaceutical compositions also may contain, optionally, suitable preservatives, such as: benzalkonium chloride; chlorobutanol; parabens and thimerosal.

The expression vector compositions may conveniently be presented in unit dosage form and may be prepared by any of the methods well-known in the art of pharmacy. All methods include the step of bringing the active agent into association with a vector which constitutes one or more accessory ingredients. The preparation may be formulated according to known methods using suitable dispersing or wetting agents and suspending agents. The sterile injectable preparation also may be a sterile injectable solution or suspension in a non-toxic parenterally acceptable diluent or solvent, for example, as a solution in 1, 3-butanediol.

Among the acceptable solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, PA.

According to a further aspect of the invention there is provided an expression vector according to the invention for use as a medicament.

According to a further aspect of the invention there is provided an expression vector according to the invention for use in the treatment of AP-4 Hereditary Spastic Paraplegias in a subject.

Preferably, said subject is a paediatric subject.

Paediatric subjects include neonates (0-28 days old), infants (1-24 months old), young children (2-6 years old) and prepubescent [7-14 years old].

In a preferred embodiment of the invention said AP-4-HSP is SPG47

In a preferred embodiment of the invention said AP-4-HSP is SPG50.

In a preferred embodiment of the invention said AP-4-HSP is SPG51.

In a preferred embodiment of the invention said AP-4-HSP is SPG52.

Spastic Paraplegia (SPG) is used interchangeably with Hereditary Spastic Paraplegia (HSP), thus SPG47 is HSP47, SPG50 is HSP50, SPG51 is HSP51 and SPG52 is HSP52.

According to a further aspect of the invention there is provided a cell transfected with an expression vector according to the invention.

In a preferred embodiment of the invention said cell is a neurone.

In a preferred embodiment of the invention said neurone is a motor neurone.

According to a further aspect of the invention there is provided a method to treat or prevent AP-4 Hereditary Spastic Paraplegias comprising administering a therapeutically effective amount of an expression vector according to the invention to prevent and/or treat Hereditary Spastic Paraplegias.

In a preferred method of the invention said AP-4-HSP is Spastic Paraplegia type 47 (SPG47).

In a preferred method of the invention said AP-4 HSP is Spastic Paraplegia type 50 (SPG50).

In a preferred method of the invention said AP-4 HSP is Spastic Paraplegia type 51 (SPG51). In a preferred method of the invention said AP-4 HSP is Spastic Paraplegia type 52 (SPG52).

According to a further aspect of the invention there is provide a method for measuring the efficacy of the treatment of hereditary spastic paraplegia in a subject wherein said subject is treated with an expression vector or the pharmaceutical composition according to the invention, said method comprising:
a) measuring the level of neurofilament L (NFL) in a biological sample obtained from the subject suffering from hereditary spastic paraplegia prior to administration of the expression vector according or the pharmaceutical composition according to the invention, and
b) comparing said levels to the levels of NFL in a biological sample obtained from the subject suffering from hereditary spastic paraplegia after the administration of the expression vector or the pharmaceutical composition according to the invention, and wherein
i) if the NFL levels are lower when compared to the levels obtained in step a) the treatment with the expression vector or composition is paused, or
ii) if the levels are substantially the same as the levels in step a) the treatment with the expression vector or composition according to the invention is continued.

In a preferred method of the invention said expression vector is selected from a group consisting of SEQ ID NO 10, 11, 12, 13, 14, 23, 24, 25, 26 and 27.

In a preferred method of the invention said sample under b) is obtained between 1, 2, 3, 4, 5 or 6 days or 1, 2, 3 or-4 weeks after administration.

According to a further aspect of the invention there is provided a method for measuring the efficacy of the treatment of hereditary spastic paraplegia in a subject suffering from hereditary spastic paraplegia wherein said subject is treated with an expression vector or pharmaceutical composition according to the invention, said method comprising:
a) measuring the level of neurofilament L (NFL) in a biological sample obtained from the subject suffering from hereditary spastic paraplegia and treated with said expression vector or composition, and
b) comparing said level with that of control subjects.

In a preferred method according to the invention, said expression vector is selected from a group consisting of SEQ ID NO 10, 11, 12, 13, 14, 23, 24, 25, 26 and 27.

In a preferred method of the invention said sample under a) is obtained between 1, 2, 3, 4, 5 or 6 days or 1, 2, 3 or-4 weeks after the treatment with the expression vector or pharmaceutical composition according to the invention

In a preferred method of the invention said method further comprises step c) wherein when said levels in a biological sample obtained from the subject suffering from hereditary spastic paraplegia are the same or lower than the NFL levels in a biological sample obtained from the control subject the treatment is effective and paused.

In a preferred method of the invention said method further comprises step c) wherein when said levels in a biological sample obtained from the subject suffering from hereditary spastic paraplegia are higher than the NFL levels in a biological sample obtained from the control subject the treatment is ineffective and the treatment with an expression vector or the pharmaceutical composition according to the invention is continued.

Throughout the description and claims of this specification, the words “comprise” and “contain” and variations of the words, for example “comprising” and “comprises”, means “including but not limited to”, and is not intended to (and does not) exclude other moieties, additives, components, integers or steps. “Consisting essentially” means having the essential integers but including integers which do not materially affect the function of the essential integers.

Throughout the description and claims of this specification, the singular encompasses the plural unless the context otherwise requires. Where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

FIG. 1—The AP4 complex and function: (A) Illustration of the AP4 heterotetramer complex, comprised of the large beta and epsilon-type adaptins (B4 and &4, termed AP4B1 and AP4E1), the medium mu-type adaptin (APu4, termed AP4M1) and the small sigma adaptin (APo4, termed AP4S1). (B) Illustration of the AP4 complex function 1) AP4 heterotetramers are recruited to the trans-Golgi network (TGN) and in turn recruit their cargo proteins, including ATG9. 2) clatherin negative vesicles bud from the TGN. 3) The AP4 complex is shed from the vesicles and recycled back to the TGN for further vesicle formation. 4) Remaining vesicles are bound by Kinesin motor proteins and transported in an anterograde direction along microtubules to the cell periphery or distal neuronal compartments. 5) ATG9 vesicles assemble to promote autophagosome formation;

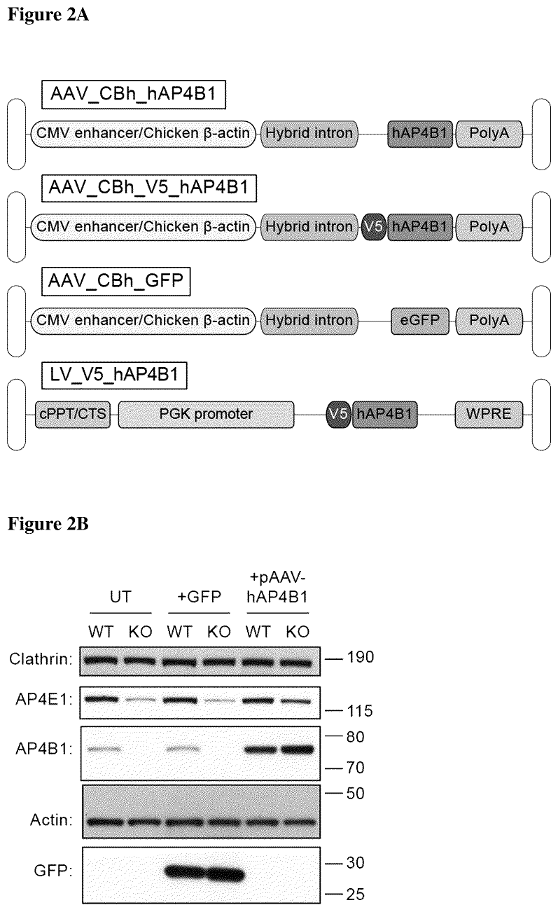

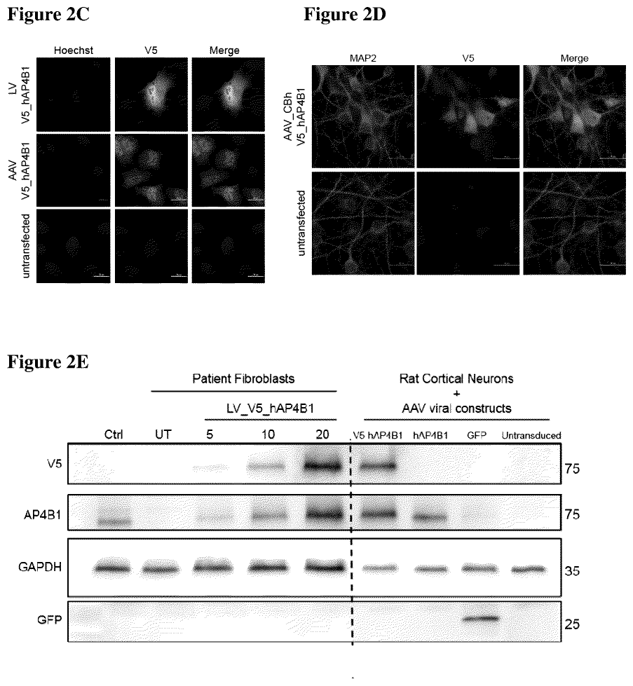

FIG. 2—Design and validation of gene therapy vectors for AP4B1 gene replacement. (A) Schematic illustration of the AAV and LV designed and already in place. (B) Representative western blot of control (WT) or AP4B1-knockout (KO) HeLa cell lysates after transfection with plasmids expressing GFP (+GFP) or hAP4B1 (+AAV-hAP4B1). Expression of hAP4B1 in KO cell lines rescues missing AP4B1 protein expression. Rescue of AP4B1 expression also restores expression of AP4E1 subunit protein levels to WT levels. (C) Validation of AAV-expressing V5 tagged AP4B1 in AP4B1−/−Hela cells. (D) Non-transgenic rat cortical neurons stained with cortical neuron marker MAP2 (Red channel) and V5 (Green channel) primary antibodies, 10 days post-treatment with either 300,000 vg/cell AAV9-V5_hAP4B1. (E) Representative western blot of non-disease control fibroblasts (Ctrl), and SPG47 patient fibroblasts both untreated (UT) and treated with increasing amounts of LV-V5_hAP4B1 showing rescue of hAP4B1 expression in patient mutant lines (left side of dashed line). Representative western blot of non-transgenic rat cortical neurons treated with 400,000 vg/cell AAV9 viral vectors: AAV9-V5_SPG47 (hAP4B1); AAV9-SPG47 (hAP4B1); AAV9-GFP; non-transduced cells;

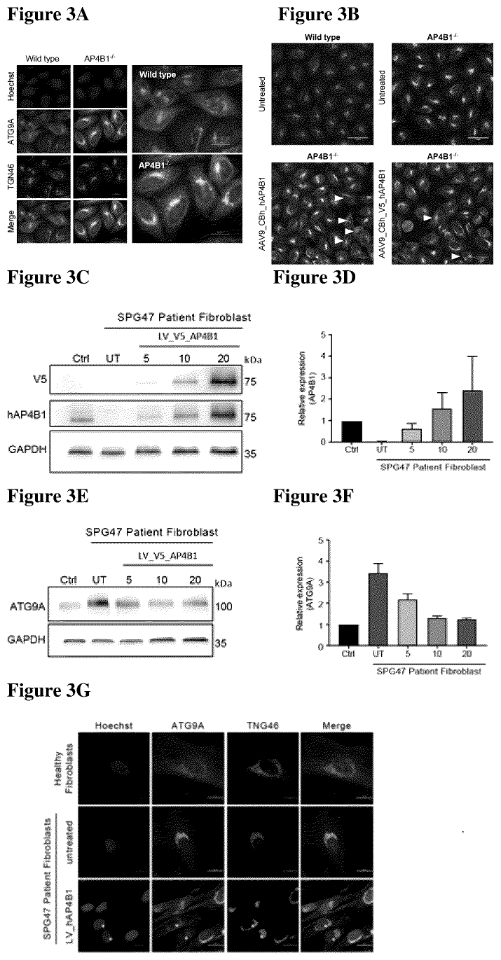

FIG. 3—Use of ATG9A as readout to assess efficacy of AB4B1 gene replacement. (A) illustration of the mislocalisation of ATG9A to the TGN trans-golgi network (TGN) in CRISPR generated Hela knockout cell model. ATG9A mislocalisation in AP4B1−/−Hela cells is rescued after transfection with AAV9 constructs encoding AP4B1 (B). Representative western blot of LV transduced patient cells shows rescue of hAP4B1 expression and detection of V5 tag (C), quantified in (D). Transduced patient cells also show rescue of ATG9A expression (E), quantified in (F). Data is presented as mean+/−standard error of the mean (SEM), n=3. Data analysed by one-way ANOVA followed by post-hoc Dunnett's multiple comparisons test with respect to Ctrl. Stars indicate p≤0.05 (*); p<0.0001 (****); ns=not significant. (G) Lentiviral vector (LV)-mediated correction of ATG9A mislocalisation in SPG47 patient primary fibroblast cells. SPG47 patient cells marked with white asterisks show rescue of mislocalised ATG9A after treatment with LV-V5_hAP4B1;

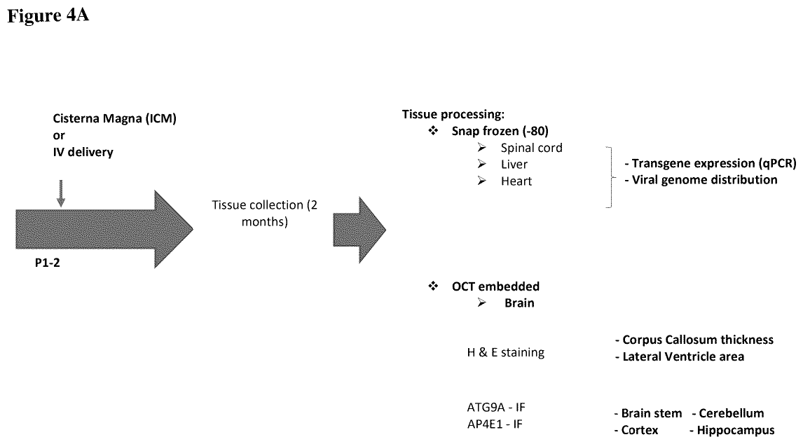

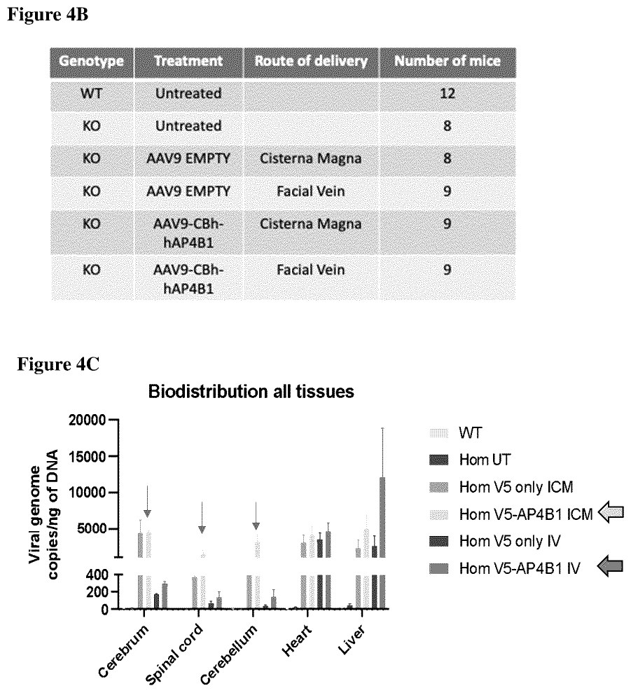

FIG. 4—Proof-of-Concept Study 1: Biochemical and anatomical assessment. Aim was to evaluate impact of AAV9-CBh-AP4B1 gene replacement on key biochemical and anatomical deficits identified in Ap4b1−/−. (A) Study design. AAV9 Injections in P1 mice as per the two main paradigms of gene therapy delivery: intra-CSF delivery through intra-cisterna magna (ICM) or intravenous (IV) delivery through facial vein. (B) Sample size used in the study. (C) Intra-cisterna magna delivery of AAV9-V5 only and AAV9-V5-hAP4B1 is superior to intravenous delivery in transducing all areas of the CNS. Viral biodistribution as determined by qPCR in cerebrum, spinal cord, and cerebellum. N=3 for each tissue, results are displayed as mean±SEM.

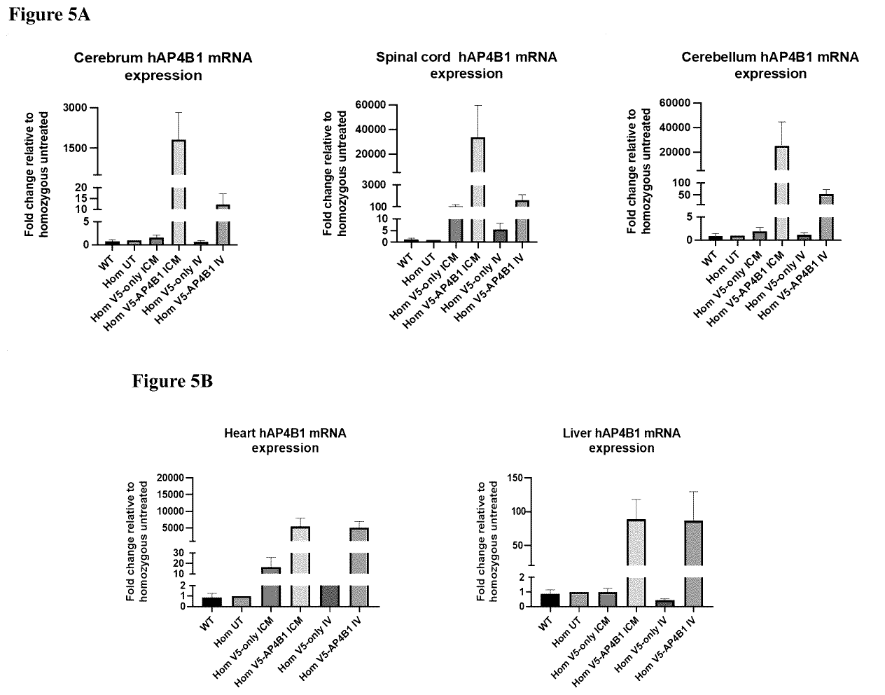

FIG. 5—Intra-cisterna magna delivery of AAV9-V5-hAP4B1 is superior to intravenous delivery in inducing hAP4B1 mRNA expression in all areas of the CNS. RT-qPCR of hAP4B1 cDNA in cerebrum, spinal cord, and cerebellum. N=3 for each tissue, results are displayed as mean±SEM.

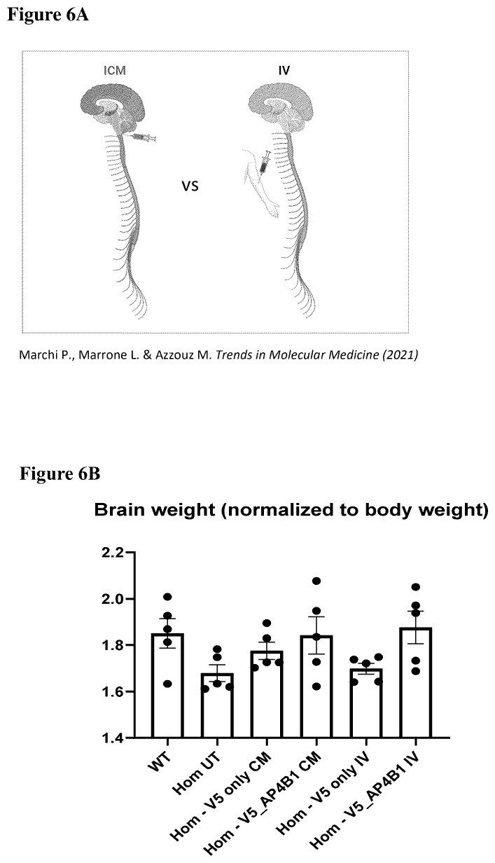

FIG. 6—AAV9-CBh-hAP4B1 gene therapy mediate rescue of brain weight deficit in Ap4b1−/− mouse model. Mice were treated with AAV9 empty control (V5) or AAV9 expressing V5 tagged hAP4B1 viruses. Hom=Ap4b1−/−.

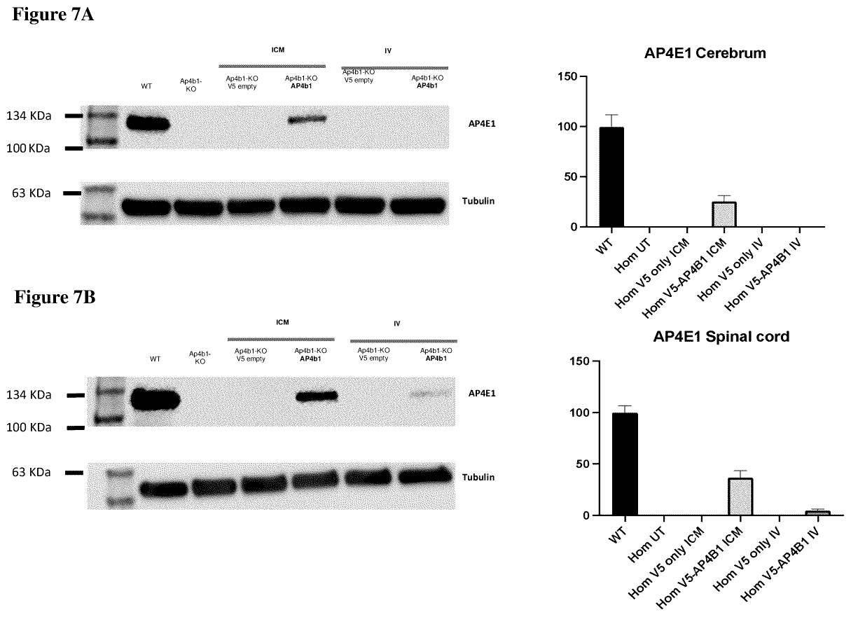

FIG. 7. ICM delivery of AAV9-V5-hAP4B1 rescues mouse AP4E1 protein levels in the CNS and is superior to intravenous delivery. Western blots of protein extracts showing rescue of mouse AP4E1 levels in the cerebrum (A), and spinal cord (B) of mice treated with ICM injection of AAV9-V5-hAP4B1. Intravenous delivery induced detectable AP4E1 rescue only in spinal cord. N=3 for each tissue, quantification data shown as mean±SEM.

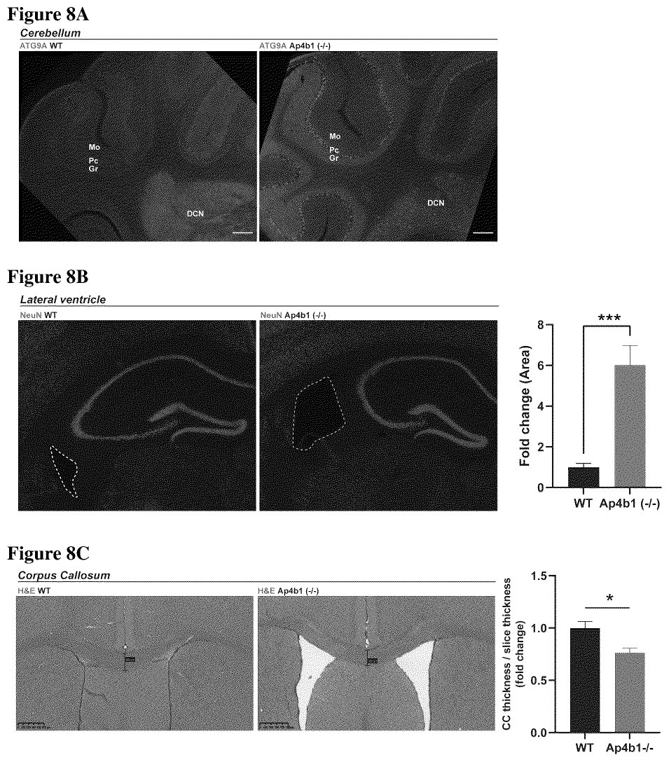

FIG. 8. Mislocalised ATG9A, enlarged lateral ventricles and reduced corpus callosum thickness in the brains of Ap4b1 (−/−) mice. Immunostaining of ATG9A in cerebellar sections shows mislocalisation and increased expression in the Purkinje cell layer and deep cerebellar nuclei of Ap4b1 (−/−) mice (A). NeuN stained coronal brain sections with lateral ventricle highlighted by dashed line in Ap4b1 (−/−) mice. Graph shows quantification of lateral ventricle area as a fold change with respect to WT animals. N=3, analysed by Kolmogorov-Smirnov test. *** p≤0.001 (B). Coronal sections stained with Hematoxylin and eosin (H&E) show reduced corpus callosum thickness in Ap4b1 (−/−) mice. Graph shows quantification of corpus thickness normalised to slice thickness as a fold change with respect to WT animals. Data shown as mean±S. E. M, n=3, analysed by Kolmogorov-Smirnov test. * p<0.05 (C). Scale bar: 150 μm (A); 500 μm (C). Mo=molecular layer, Pc=Purkinje cell layer, Gr=granular cell layer, DCN=deep cerebellar nuclei, H&E=hematoxylin and eosin, ATG9A=autophagy-related protein 9A, CC=corpus callosum.

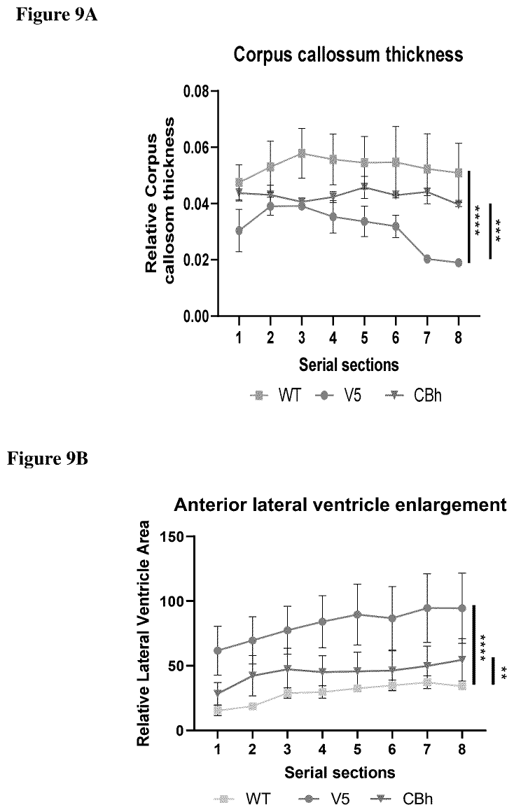

FIG. 9. AAV9-mediated AP4B1 gene replacement led to the rescue of (A) corpus callosum thickness and (B) enlarged lateral ventricles in Ap4b1−/−mouse model.

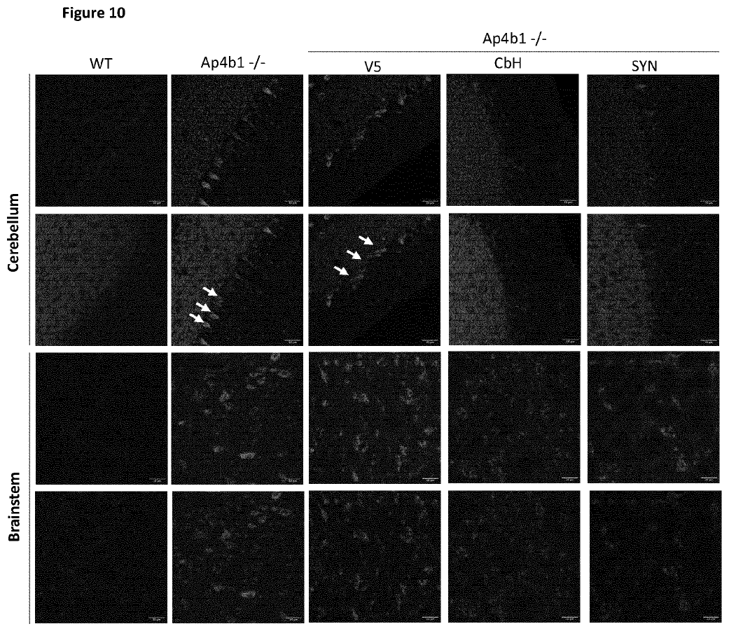

FIG. 10. AAV9 gene replacement of AP4B1 in SPG47 mouse model restore normal localisation of ATG9A in the cerebellum and brainstem.

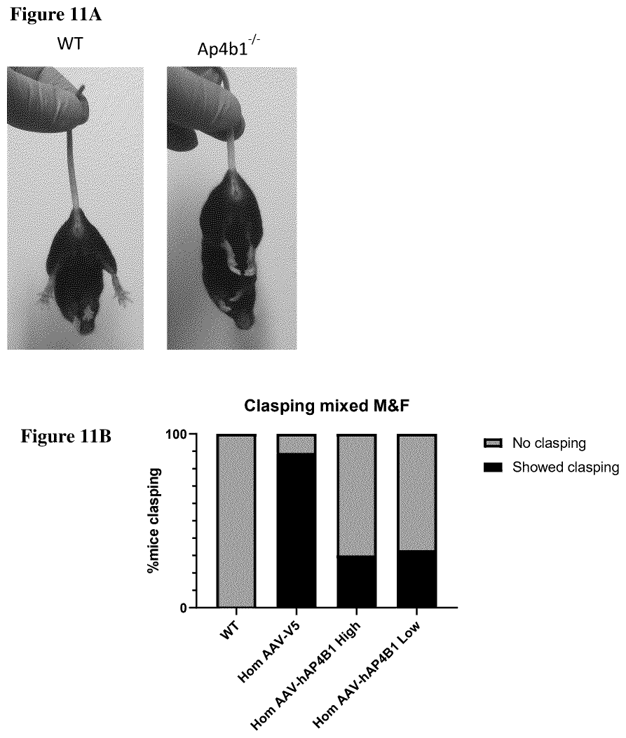

FIG. 11. Ap4b1 (−/−) mice exhibit hindlimb clasping. Representative non-clasping (WT) and clasping (Ap4b1 (−/−)) images are shown (A). Percentage of WT and Ap4b1 (−/−) mice who show hindlimb clasping at 3 months of age (B). AAV9-CBh-AP4B1 treatment reduces clasping phenotype in Ap4b1−/−.

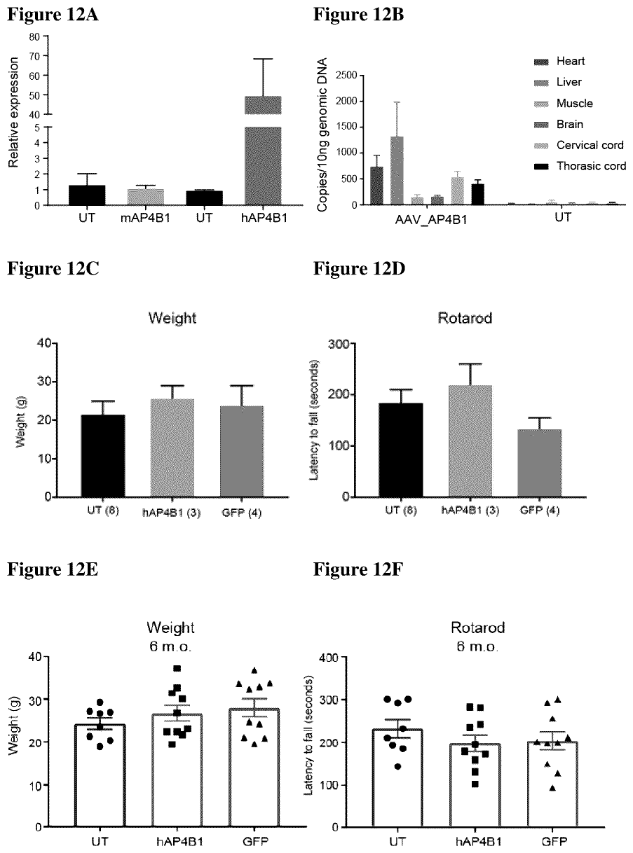

FIG. 12. Pilot safety study in wild type mice. Mice treated with AAV9-CBh-AP4B1 delivered via ICM show no sign of side effects up to 6 months. mRNA transgene expression (A) and distribution of viral genome (B) were assessed in the brain, spinal cord and peripheral organs. AAV gene therapy had no adverse effect on body weight and functional motor coordination in mice was measured by rotarod at 4 weeks (B) and 6 months post treatment (C). n=5

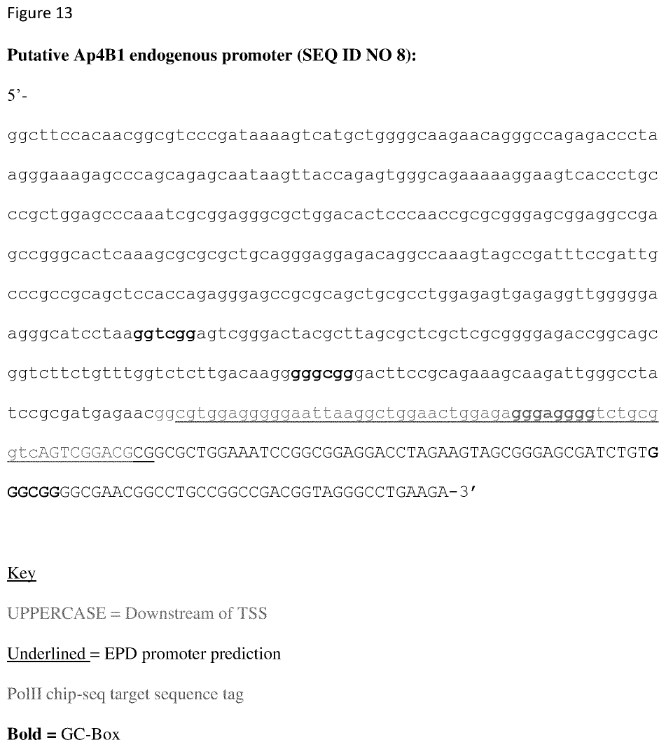

FIG. 13 describes AP4B1 endogenous promoter sequence;

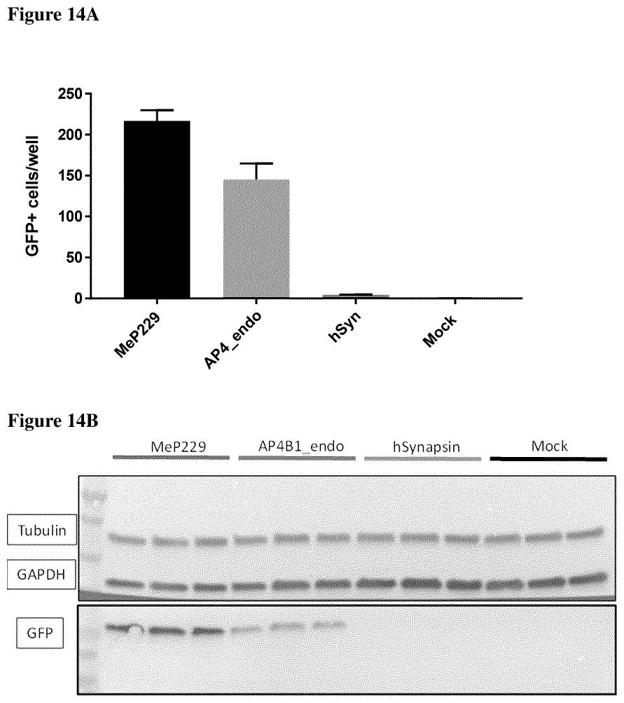

FIG. 14 GFP expression in Hela cells under the control of the MeP229, AP4 and hSyn promoters. The term “mock” indicates the control with no expression of GFP. As shown, expression under all three promoters was detected. Experiment was performed using three replicas for each sample. Data presented as mean±SD.

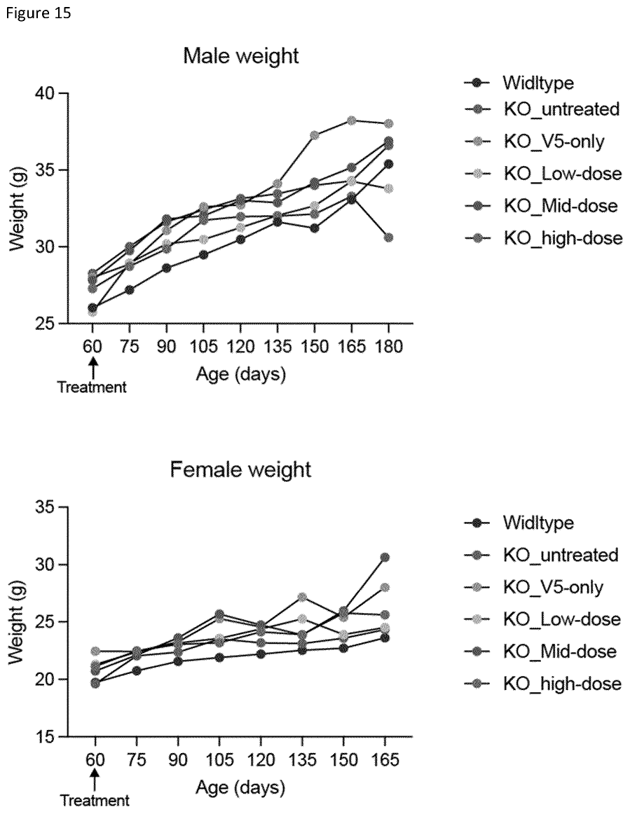

FIG. 15: Age-dependant weight gain in AAV9-CBh-hAP4B1 treated mice up to 180 days. Treatment ˜p60. (Females have only reached 165 days).

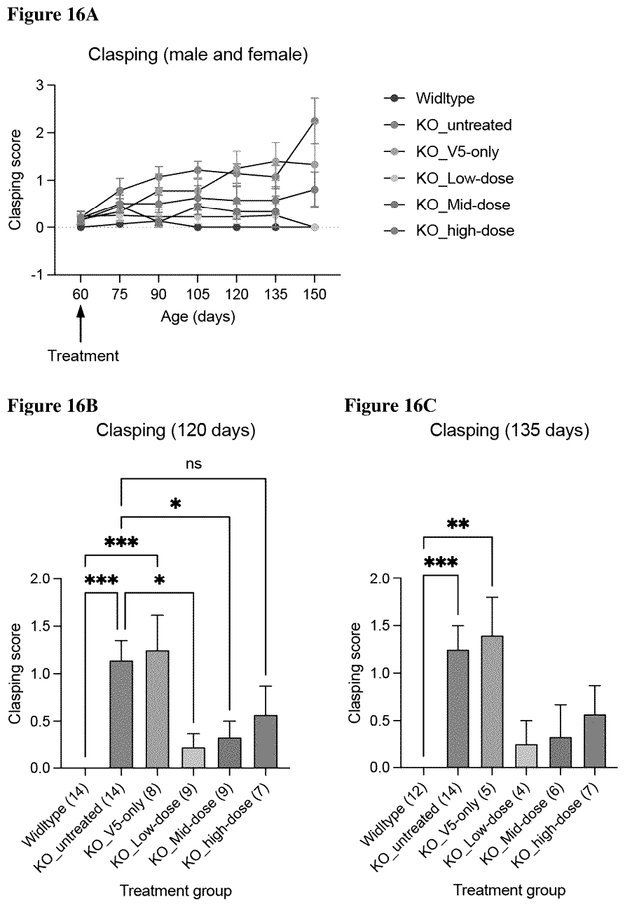

FIG. 16: Hind limb clasping of treated mice. A. shows a progression of clasping severity over time in SPG47 mice (ap4b1−/−) untreated and V5_only treated. Wildtype mice do not show progression in hind limb clasping throughout this period. All 3 treatment groups are showing a reduction in hind limb progression severity. B and C are extracted clasping data from one timepoint, 120 days of age or 135 days of age respectively. Both graphs clearly show the reduction in hind limb clasping severity with all treatments.

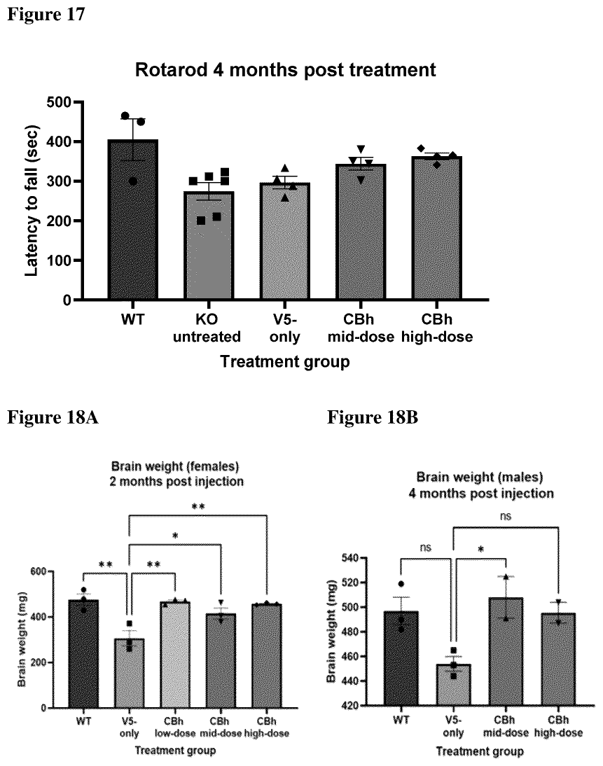

FIG. 17: Rotarod latency to fall at 4 months post treatment (˜p180).

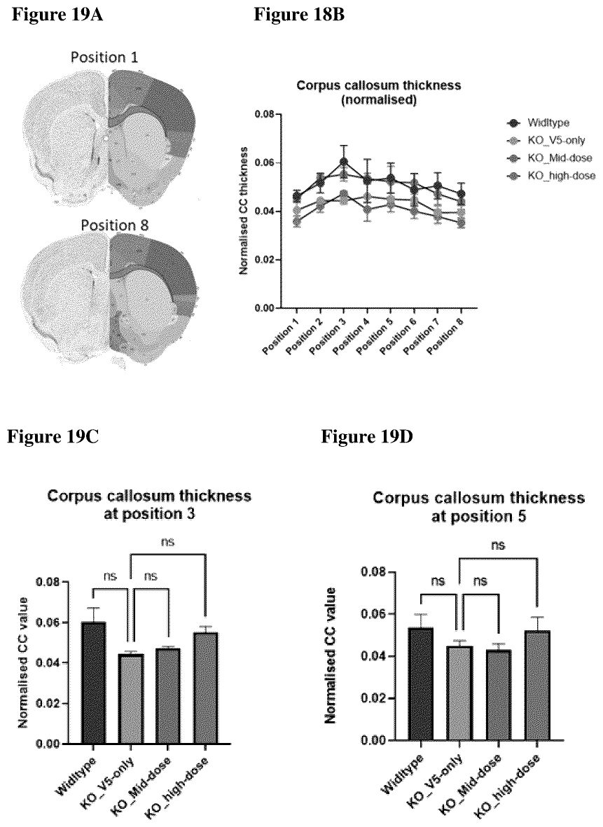

FIG. 18: Brain weight after brain extraction. Females at 2 months post injection (˜P120). Males at 4 months post injection (˜P180).

FIG. 19: Preliminary corpus callosum thinning analysis 4 months post injection (males only). A Diagram to show the corpus callosum (CC) in a relative coronal position. Every second section is taken between position 1 and position 8 for CC analysis. B displays the variance in CC width as you go through the brain. SPG47 V5_only cohort shows a reduced CC thickness compared to wildtype CC thickness, and this data suggests this phenotype can be rescued with the high-dose treatment. CC width measurements here are normalised to each brain section. C and D are data extracted from a single position showing the same result.

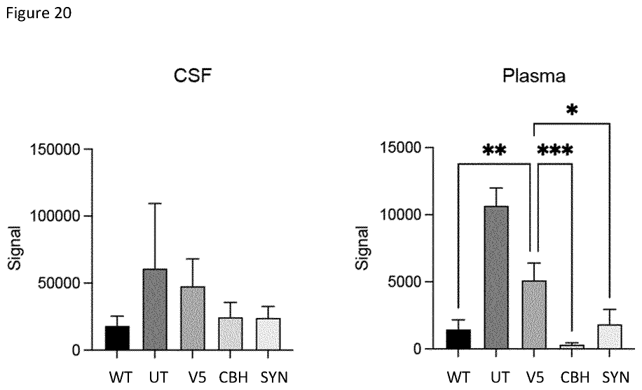

FIG. 20 Impact of AAV9-AP4B1 gene replacement on neurofilament L (NFL) levels in cerebrospinal fluid (CSF) and Plasma. Ap4b1−/−mice were treated with AAV9-hAP4B1 vectors via cisterna magna at P1. WT: wild type, UT: untreated Ap4b1−/−, V5: Ap4b1−/−treated with control empty vector, CBH: Ap4b1−/−treated with AAV9-CBh-AP4B1, SYN: Ap4b1−/−treated with AAV9-Synapsin 1-AP4B1.

SEQ ID NO SUMMARY TABLE

SEQ ID NO 1: CMV ENHANCER
cgttacataacttacggtaaatggcccgcctggctgaccgcccaacgacccccgcccattgacgtcaatagtaacgccaa
tagggactttccattgacgtcaatgggtggagtatttacggtaaactgcccacttggcagtacatcaagtgtatcatatg
ccaagtacgccccctattgacgtcaatgacggtaaatggcccgcctggcattatgcccagtacatgaccttatgggactt
tcctacttggcagtacatctacgtattagtcatcgctattaccatgg
SEQ ID NO 2: HYBRID INTRON
ggagtcgctgcgacgctgccttcgccccgtgccccgctccgccgccgcctcgcgccgcccgccccggctctgactgaccg
cgttactcccacaggtgagcgggcgggacggcccttctcctccgggctgtaattagctgagcaagaggtaagggtttaag
ggatggttggttggtggggtattaatgtttaattacctggagcacctgcctgaaatcactttttttcag
SEQ ID NO 3: CHICKEN BETA ACTIN PROMOTER
tcgaggtgagccccacgttctgcttcactctccccatctcccccccctccccacccccaattttgtatttatttattttt
taattattttgtgcagcgatgggggcggggggggggggggggcgcgcgccaggcggggcggggcggggcgaggggcgggg
cggggcgaggcggagaggtgcggcggcagccaatcagagcggcgcgctccgaaagtttccttttatggcgaggcggcggc
ggcggcggccctataaaaagcgaagcgcgcggcgggcg
SEQ ID NO 4: GROWTH HORMONE POLYADENYLATION SIGNAL
gggtggcatccctgtgacccctccccagtgcctctcctggccctggaagttgccactccagtgcccaccagccttgtcct
aataaaattaagttgcatcattttgtctgactaggtgtccttctataatattatggggtggaggggggtggtatggagca
aggggcaagttgggaagacaacctgtagggcctgcggggtctattgggaaccaagctggagtgcagtggcacaatcttgg
ctcactgcaatctccgcctcctgggttcaagcgattctcctgcctcagcctcccgagttgttgggattccaggcatgcat
gaccaggctcagctaattttttgttttttggtagagacggggtttcaccatattggccaggctggtctccaactcctaat
ctcaggtgatctacccaccttggcctcccaaattgctgggattacaggcgtgaaccactgctcccttccctgtcctt
SEQ ID NO 5 JeT promoter
gggcggagttagggcggagccaatcagcgtgcgccgttccgaaagttgccttttatggctgggcggagaatgggcggtga
acgccgatgattatataaggacgcgccgggtgtggcacagctagttccgtcgcagccgggatttgggtcgcggttcttgt
ttgt
SEQ ID NO 6: hSyn Promoter
agtgcaagtgggttttaggaccaggatgaggcggggtgggggtgcctacctgacgaccgaccccgacccactggacaagc
acccaacccccattccccaaattgcgcatcccctatcagagagggggaggggaaacaggatgcggcgaggcgcgtgcgca
ctgccagcttcagcaccgcggacagtgccttcgcccccgcctggcggcgcgcgccaccgccgcctcagcactgaaggcgc
gctgacgtcactcgccggtcccccgcaaactccccttcccggccaccttggtcgcgtccgcgccgccgccggcccagccg
gaccgcaccacgcgaggcgcgagataggggggcacgggcgcgaccatctgcgctgcggcgccggcgactcagcgctgcct
cagtctgcggtgggcagcggaggagtcgtgtcgtgcctgagagcgcag
SEQ ID NO 7: MeP 229 promoter
CAATTGAGGGCGTCACCGCTAAGGCTCCGCCCCAGCCTGGGCTCCACAACCAATGAAGGGTAAT
CTCGACAAAGAGCAAGGGGTGGGGCGCGGGCGCGCAGGTGCAGCAGCACACAGGCTGGTCGG
GAGGGCGGGGCGCGACGTCTGCCGTGCGGGGTCCCGGCATCGGTTGCGCGCGCGCTCCCTCCTC
TCGGAGAGAGGGCTGTGGTAAAACCCGTCCGGAAA
SEQ ID NO 8: AP4B1 promoter
ggcttccacaacggcgtcccgataaaagtcatgctggggcaagaacagggccagagaccctaagggaaagagcccagcag
agcaataagttaccagagtgggcagaaaaaggaagtcaccctgcccgctggagcccaaatcgcggagggcgctggacact
cccaaccgcgcgggagcggaggccgagccgggcactcaaagcgcgcgctgcagggaggagacaggccaaagtagccgatt
tccgattgcccgccgcagctccaccagagggagccgcgcagctgcgcctggagagtgagaggttgggggaagggcatcct
aaggtcggagtcgggactacgcttagcgctcgctcgcggggagaccggcagcggtcttctgtttggtctcttgacaaggg
ggcgggacttccgcagaaagcaagattgggcctatccgcgatgagaacggcgtggagggggaattaaggctggaactgga
gagggaggggtctgcggtcagtcggacgcggcgctggaaatccggcggaggacctagaagtagcgggagcgatctgtggg
cggggcgaacggcctgccggccgacggtagggcctgaaga
SEQ ID NO 9: CBh promoter (full)
cgttacataacttacggtaaatggcccgcctggctgaccgcccaacgacccccgcccattgacgtcaatagtaacgccaa
tagggactttccattgacgtcaatgggtggagtatttacggtaaactgcccacttggcagtacatcaagtgtatcatatg
ccaagtacgccccctattgacgtcaatgacggtaaatggcccgcctggcattatgcccagtacatgaccttatgggactt
tcctacttggcagtacatctacgtattagtcatcgctattaccatggtcgaggtgagccccacgttctgcttcactctcc
ccatctcccccccctccccacccccaattttgtatttatttattttttaattattttgtgcagcgatgggggcggggggg
gggggggggcgcgcgccaggcggggcggggcggggcgaggggcggggcggggcgaggcggagaggtgcggcggcagccaa
tcagagcggcgcgctccgaaagtttccttttatggcgaggcggcggcggcggcggccctataaaaagcgaagcgcgcggc
gggcgggagtcgctgcgacgctgccttcgccccgtgccccgctccgccgccgcctcgcgccgcccgccccggctctgact
gaccgcgttactcccacaggtgagcgggcgggacggcccttctcctccgggctgtaattagctgagcaagaggtaagggt
ttaagggatggttggttggtggggtattaatgtttaattacctggagcacctgcctgaaatcactttttttcag
SEQ ID NO 10: pAAV-AP4B1endog-hAP4B1-Kan
cctgcaggcagctgcgcgctcgctcgctcactgaggccgcccgggcaaagcccgggcgtcgggcgacctttggtcgcccg
gcctcagtgagcgagcgagcgcgcagagagggagtggccaactccatcactaggggttcctgcggccgcacgcgtccggc
ttccacaacggcgtcccgataaaagtcatgctggggcaagaacagggccagagaccctaagggaaagagcccagcagagc
aataagttaccagagtgggcagaaaaaggaagtcaccctgcccgctggagcccaaatcgcggagggcgctggacactccc
aaccgcgcgggagcggaggccgagccgggcactcaaagcgcgcgctgcagggaggagacaggccaaagtagccgatttcc
gattgcccgccgcagctccaccagagggagccgcgcagctgcgcctggagagtgagaggttgggggaagggcatcctaag
gtcggagtcgggactacgcttagcgctcgctcgcggggagaccggcagcggtcttctgtttggtctcttgacaagggggc
gggacttccgcagaaagcaagattgggcctatccgcgatgagaacggcgtggagggggaattaaggctggaactggagag
ggaggggtctgcggtcagtcggacgcggcgctggaaatccggcggaggacctagaagtagcgggagcgatctgtgggcgg
ggcgaacggcctgccggccgacggtagggcctgaagaggaattccccggggatcctctagagtcgacgccaccatgccgt
accttggctccgaggacgtggtgaaggagctgaagaaggctctgtgcaatcctcacattcaagctgataggctgcgctac
cggaatgtcatccagcgagtgattaggtacatgactcaaggcttggacatgtctggtgtttttatggaaatggtgaaggc
cagtgccactgtagatattgtccagaagaagttggtttatctgtacatgtgcacatatgctcccctgaaaccagatctgg
ctctcctggccatcaatacgctgtgcaaagactgctcagaccccaatccaatggtgcgagggctggcgttacggagcatg
tgtagcctcaggatgcctggtgtgcaggagtatatacaacagcctattctcaatggtctgcgggataaggcttcatatgt
caggagagtggcagtccttggatgtgccaagatgcataatcttcatggagactctgaagtagatggtgccctggtaaatg
aattatacagtttgctgcgtgaccaggatccaattgtagttgtgaactgcttgaggtctctagaggaaattctgaaacag
gaaggaggcgttgtcatcaataagcccattgctcaccatctcttaaatcgaatgtcaaaactggaccaatggggccaggc
tgaagtattgaactttctgctacgctaccaaccccgcagtgaggaagaactatttgacattctcaatctgttggatagtt
tcctcaagagcagtagcccaggtgtggtgatgggagctaccaaactttttctgatcttggcaaaaatgtttccccacgta
caaactgatgtccttgtgcgggtcaagggacctttgctagctgcctgttcttcagagagccgtgagctctgttttgttgc
tctttgtcatgtacgccagatcttgcatagtttaccaggtcactttagcagccactacaaaaagtttttttgctcctact
cggagccccactacatcaaactacagaaagtggaggtgctgtgtgaactggtgaacgatgagaatgtgcagcaggtgcta
gaggagcttcgagggtactgcacggatgtgtctgcggactttgcacaggctgccatctttgccataggtggcattgccag
gacttacacagatcaatgtgttcagattttaacagagttgctgggtcttcgacaagagcacattaccacagtggtggtgc
agactttccgagacctggtttggttgtgtcctcagtgtactgaagctgtatgtcaggccctgcccggctgtgaagagaac
attcaagatagtgaggggaagcaagcacttatttggctacttggtgtccatggggaaagaattcctaatgctccttatgt
gttagaggactttgttgagaatgtgaagtcggaaacatttccagctgttaagatggagctgctcactgctttgctgcgcc
ttttcctctcccgacctgctgagtgccaggacatgctaggacgtttgttgtattactgcatagaggaagaaaaagatatg
gctgtacgggaccgaggtctcttctattatcgcctcctcttagttggcattgatgaagttaagcggattctgtgtagccc
taaatctgaccctactcttggacttttggaggatccggcagaaagacctgtgaatagctgggcctcagacttcaacacac
tggtgccagtgtatggcaaagcccactgggcaactatctctaaatgccagggggcagagcgttgtgacccagagcttcct
aaaacttcatcctttgccgcatcaggacccttgattcctgaagagaacaaggagagggtacaagaactccctgattctgg
agccctcatgctagtccccaatcgccagcttactgctgattattttgagaaaacttggcttagccttaaagttgctcatc
agcaagtgttgccttggcggggagaattccatcctgacaccctccagatggctcttcaagtagtgaacatccagaccatc
gcaatgagtagggctgggtctcggccatggaaagcatacctcagtgctcaggatgatactggctgtctgttcttaacaga
actgctattggagcctggaaactcagaaatgcagatctctgtgaaacaaaatgaagcaagaacggagacgctgaatagtt
ttatttctgtattagaaactgtgattggaacaattgaagaaataaaatcataagcggccgcaaagcttgcctcgagagat
ctacgggtggcatccctgtgacccctccccagtgcctctcctggccctggaagttgccactccagtgcccaccagccttg
tcctaataaaattaagttgcatcattttgtctgactaggtgtccttctataatattatggggtggaggggggtggtatgg
agcaaggggcaagttgggaagacaacctgtagggcctgcggggtctattgggaaccaagctggagtgcagtggcacaatc
ttggctcactgcaatctccgcctcctgggttcaagcgattctcctgcctcagcctcccgagttgttgggattccaggcat
gcatgaccaggctcagctaatttttgtttttttggtagagacggggtttcaccatattggccaggctggtctccaactcc
taatctcaggtgatctacccaccttggcctcccaaattgctgggattacaggcgtgaaccactgctcccttccctgtcct
tctgattttgtaggtaaccacgtgcggaccgagcggccgcaggaacccctagtgatggagttggccactccctctctgcg
cgctcgctcgctcactgaggccgggcgaccaaaggtcgcccgacgcccgggctttgcccgggcggcctcagtgagcgagc
gagcgcgcagctgcctgcaggGGCGCCTGATGCGGTATTTTCTCCTTACGCATCTGTGCGGTATTTCACACC
GCATACGTCAAAGCAACCATAGTACGCGCCCTGTAGCGGCGCATTAAGCGCGGCGGGTGTGGT
GGTTACGCGCAGCGTGACCGCTACACTTGCCAGCGCCTTAGCGCCCGCTCCTTTCGCTTTCTTCC
CTTCCTTTCTCGCCACGTTCGCCGGCTTTCCCCGTCAAGCTCTAAATCGGGGGCTCCCTTTAGGG
TTCCGATTTAGTGCTTTACGGCACCTCGACCCCAAAAAACTTGATTTGGGTGATGGTTCACGTAG
TGGGCCATCGCCCTGATAGACGGTTTTTCGCCCTTTGACGTTGGAGTCCACGTTCTTTAATAGTG
GACTCTTGTTCCAAACTGGAACAACACTCAACTCTATCTCGGGCTATTCTTTTGATTTATAAGGG
ATTTTGCCGATTTCGGTCTATTGGTTAAAAAATGAGCTGATTTAACAAAAATTTAACGCGAATTT
TAACAAAATATTAACGTTTACAATTTTATGGTGCACTCTCAGTACAATCTGCTCTGATGCCGCAT
AGTTAAGCCAGCCCCGACACCCGCCAACACCCGCTGACGCGCCCTGACGG
GCTTGTCTGCTCCCGGCATCCGCTTACAGACAAGCTGTGACCGTCTCCGGGAGCTGCATGTGTC
AGAGGTTTTCACCGTCATCACCGAAACGCGCGAGACGAAAGGGCCTCGTGATACGCCTATTTTT
ATAGGTTAATGTCATGATAATAATGGTTTCTTAGACGTCCTGGCCCGTGTCTCAAAATCTCTGAT
GTTACATTGCACAAGATAAAAATATATCATCATGAACAATAAAACTGTCTGCTTACATAAACAG
TAATACAAGGGGTGTTATGAGCCATATTCAACGGGAAACGTCGAGGCCGCGATTAAATTCCAAC
ATGGATGCTGATTTATATGGGTATAAATGGGCTCGCGATAATGTCGGGCAATCAGGTGCGACAA
TCTATCGCTTGTATGGGAAGCCCGATGCGCCAGAGTTGTTTCTGAAACATGGCAAAGGTAGCGT
TGCCAATGATGTTACAGATGAGATGGTCAGACTAAACTGGCTGACGGAATTTATGCCTCTTCCG
ACCATCAAGCATTTTATCCGTACTCCTGATGATGCATGGTTACTCACCACTGCGATCCCCGGAAA
AACAGCATTCCAGGTATTAGAAGAATATCCTGATTCAGGTGAAAATATTGTTGATGCGCTGG
CAGTGTTCCTGCGCCGGTTGCATTCGATTCCTGTTTGTAATTGTCCTTTTAACAGCGATCGCGTAT
TTCGTCTCGCTCAGGCGCAATCACGAATGAATAACGGTTTGGTTGATGCGAGTGATTTTGATGA
CGAGCGTAATGGCTGGCCTGTTGAACAAGTCTGGAAAGAAATGCATAAACTTTTGCCATTCTCA
CCGGATTCAGTCGTCACTCATGGTGATTTCTCACTTGATAACCTTATTTTTGACGAGGGGAAATT
AATAGGTTGTATTGATGTTGGACGAGTCGGAATCGCAGACCGATACCAGGATCTTGCCATCCTA
TGGAACTGCCTCGGTGAGTTTTCTCCTTCATTACAGAAACGGCTTTTTCAAAAATATGGTATTGA
TAATCCTGATATGAATAAATTGCAGTTTCATTTGATGCTCGATGAGTTTTTCTAATCAGAATTGG
TTAATTGGTTGTAACACTGGCAGAGCATTACGCTGACTTGACGGGACGGCGCAAGCTCATGACC
AAAATCCCTTAACGTGAGTTACGCGTGAAGATCCTTTTTGATAATCTCATGACCAAAATCCCTTA
ACGTGAGTTTTCGTTCCACTGAGCGTCAGACCCCGTAGAAAAGATCAAAGGATCTTCTTGAGAT
CCTTTTTTTCTGCGCGTAATCTGCTGCTTGCAAACAAAAAAACCACCGCTACCAGCGGTGGTTTG
TTTGCCGGATCAAGAGCTACCAACTCTTTTTCCGAAGGTAACTGGCTTCAGCAGAGCGCAGATA
CCAAATACTGTTCTTCTAGTGTAGCCGTAGTTAGGCCACCACTTCAAGAACTCTGTAGCACCGCC
TACATACCTCGCTCTGCTAATCCTGTTACCAGTGGCTGCTGCCAGTGGCGATAAGTCGTGTCTTA
CCGGGTTGGACTCAAGACGATAGTTACCGGATAAGGCGCAGCGGTCGGGCTGAACGGGGGGTT
CGTGCACACAGCCCAGCTTGGAGCGAACGACCTACACCGAACTGAGATACCTACAGCGTGAGC
TATGAGAAAGCGCCACGCTTCCCGAAGGGAGAAAGGCGGACAGGTATCCGGTAAGCGGCAGGG
TCGGAACAGGAGAGCGCACGAGGGAGCTTCCAGGGGGAAACGCCTGGTATCTTTATAGTCCTGT
CGGGTTTCGCCACCTCTGACTTGAGCGTCGATTTTTGTGATGCTCGTCAGGGGGGCGGAGCCTAT
GGAAAAACGCCAGCAACGCGGCCTTTTTACGGTTCCTGGCCTTTTGCTGGCCTTTTGCTCACATG
T
SEQ ID NO 11: pAAV-CBH-hAP4B1-Kan
cctgcaggcagctgcgcgctcgctcgctcactgaggccgcccgggcaaagcccgggcgtcgggcgacctttggtcgcccg
gcctcagtgagcgagcgagcgcgcagagagggagtggccaactccatcactaggggttcctgcggccgcacgcgtcccgt
tacataacttacggtaaatggcccgcctggctgaccgcccaacgacccccgcccattgacgtcaatagtaacgccaatag
ggactttccattgacgtcaatgggtggagtatttacggtaaactgcccacttggcagtacatcaagtgtatcatatgcca
agtacgccccctattgacgtcaatgacggtaaatggcccgcctggcattatgcccagtacatgaccttatgggactttcc
tacttggcagtacatctacgtattagtcatcgctattaccatggtcgaggtgagccccacgttctgcttcactctcccca
tctcccccccctccccacccccaattttgtatttatttattttttaattattttgtgcagcgatgggggcgggggggggg
ggggggcgcgcgccaggcggggcggggcggggcgaggggcggggcggggcgaggcggagaggtgcggcggcagccaatca
gagcggcgcgctccgaaagtttccttttatggcgaggcggcggcggcggcggccctataaaaagcgaagcgcgcggcggg
cgggagtcgctgcgacgctgccttcgccccgtgccccgctccgccgccgcctcgcgccgcccgccccggctctgactgac
cgcgttactcccacaggtgagcgggcgggacggcccttctcctccgggctgtaattagctgagcaagaggtaagggttta
agggatggttggttggtggggtattaatgtttaattacctggagcacctgcctgaaatcactttttttcagggaattccc
cggggatcctctagagtcgacgccaccatgccgtaccttggctccgaggacgtggtgaaggagctgaagaaggctctgtg
caatcctcacattcaagctgataggctgcgctaccggaatgtcatccagcgagtgattaggtacatgactcaaggcttgg
acatgtctggtgtttttatggaaatggtgaaggccagtgccactgtagatattgtccagaagaagttggtttatctgtac
atgtgcacatatgctcccctgaaaccagatctggctctcctggccatcaatacgctgtgcaaagactgctcagaccccaa
tccaatggtgcgagggctggcgttacggagcatgtgtagcctcaggatgcctggtgtgcaggagtatatacaacagccta
ttctcaatggtctgcgggataaggcttcatatgtcaggagagtggcagtccttggatgtgccaagatgcataatcttcat
ggagactctgaagtagatggtgccctggtaaatgaattatacagtttgctgcgtgaccaggatccaattgtagttgtgaa
ctgcttgaggtctctagaggaaattctgaaacaggaaggaggcgttgtcatcaataagcccattgctcaccatctcttaa
atcgaatgtcaaaactggaccaatggggccaggctgaagtattgaactttctgctacgctaccaaccccgcagtgaggaa
gaactatttgacattctcaatctgttggatagtttcctcaagagcagtagcccaggtgtggtgatgggagctaccaaact
ttttctgatcttggcaaaaatgtttccccacgtacaaactgatgtccttgtgcgggtcaagggacctttgctagctgcct
gttcttcagagagccgtgagctctgttttgttgctctttgtcatgtacgccagatcttgcatagtttaccaggtcacttt
agcagccactacaaaaagtttttttgctcctactcggagccccactacatcaaactacagaaagtggaggtgctgtgtga
actggtgaacgatgagaatgtgcagcaggtgctagaggagcttcgagggtactgcacggatgtgtctgcggactttgcac
aggctgccatctttgccataggtggcattgccaggacttacacagatcaatgtgttcagattttaacagagttgctgggt
cttcgacaagagcacattaccacagtggtggtgcagactttccgagacctggtttggttgtgtcctcagtgtactgaagc
tgtatgtcaggccctgcccggctgtgaagagaacattcaagatagtgaggggaagcaagcacttatttggctacttggtg
tccatggggaaagaattcctaatgctccttatgtgttagaggactttgttgagaatgtgaagtcggaaacatttccagct
gttaagatggagctgctcactgctttgctgcgccttttcctctcccgacctgctgagtgccaggacatgctaggacgttt
gttgtattactgcatagaggaagaaaaagatatggctgtacgggaccgaggtctcttctattatcgcctcctcttagttg
gcattgatgaagttaagcggattctgtgtagccctaaatctgaccctactcttggacttttggaggatccggcagaaaga
cctgtgaatagctgggcctcagacttcaacacactggtgccagtgtatggcaaagcccactgggcaactatctctaaatg
ccagggggcagagcgttgtgacccagagcttcctaaaacttcatcctttgccgcatcaggacccttgattcctgaagaga
acaaggagagggtacaagaactccctgattctggagccctcatgctagtccccaatcgccagcttactgctgattatttt
gagaaaacttggcttagccttaaagttgctcatcagcaagtgttgccttggcggggagaattccatcctgacaccctcca
gatggctcttcaagtagtgaacatccagaccatcgcaatgagtagggctgggtctcggccatggaaagcatacctcagtg
ctcaggatgatactggctgtctgttcttaacagaactgctattggagcctggaaactcagaaatgcagatctctgtgaaa
caaaatgaagcaagaacggagacgctgaatagttttatttctgtattagaaactgtgattggaacaattgaagaaataaa
atcataagcggccgcaaagcttgcctcgagagatctacgggtggcatccctgtgacccctccccagtgcctctcctggcc
ctggaagttgccactccagtgcccaccagccttgtcctaataaaattaagttgcatcattttgtctgactaggtgtcctt
ctataatattatggggtggaggggggtggtatggagcaaggggcaagttgggaagacaacctgtagggcctgcggggtct
attgggaaccaagctggagtgcagtggcacaatcttggctcactgcaatctccgcctcctgggttcaagcgattctcctg
cctcagcctcccgagttgttgggattccaggcatgcatgaccaggctcagctaatttttgtttttttggtagagacgggg
tttcaccatattggccaggctggtctccaactcctaatctcaggtgatctacccaccttggcctcccaaattgctgggat
tacaggcgtgaaccactgctcccttccctgtccttctgattttgtaggtaaccacgtgcggaccgagcggccgcaggaac
ccctagtgatggagttggccactccctctctgcgcgctcgctcgctcactgaggccgggcgaccaaaggtcgcccgacgc
ccgggctttgcccgggcggcctcagtgagcgagcgagcgcgcagctgcctgcaggGGCGCCTGATGCGGTATTTTCTCCT
TACGCATCTGTGCGGTATTTCACACCGCATACGTCAAAGCAACCATAGTACGCGCCCTGTAGCG
GCGCATTAAGCGCGGCGGGTGTGGTGGTTACGCGCAGCGTGACCGCTACACTTGCCAGCGCCTT
AGCGCCCGCTCCTTTCGCTTTCTTCCCTTCCTTTCTCGCCACGTTCGCCGGCTTTCCCCGTCAAGC
TCTAAATCGGGGGCTCCCTTTAGGGTTCCGATTTAGTGCTTTACGGCACCTCGACCCCAAAAAAC
TTGATTTGGGTGATGGTTCACGTAGTGGGCCATCGCCCTGATAGACGGTTTTTCGCCCTTTGACG
TTGGAGTCCACGTTCTTTAATAGTGGACTCTTGTTCCAAACTGGAACAACACTCAACTCTATCTC
GGGCTATTCTTTTGATTTATAAGGGATTTTGCCGATTTCGGTCTATTGGTTAAAAAATGAGCTGA
TTTAACAAAAATTTAACGCGAATTTTAACAAAATATTAACGTTTACAATTTTATGGTGCACTCTC
AGTACAATCTGCTCTGATGCCGCATAGTTAAGCCAGCCCCGACACCCGCCAACACCCGCTGACG
CGCCCTGACGGGCTTGTCTGCTCCCGGCATCCGCTTACAGACAAGCTGTGACCGTCTCCGGGAG
CTGCATGTGTCAGAGGTTTTCACCGTCATCACCGAAACGCGCGAGACGAAAGGGCCTCGTGATA
CGCCTATTT
TTATAGGTTAATGTCATGATAATAATGGTTTCTTAGACGTCCTGGCCCGTGTCTCAAAATCTCTG
ATGTTACATTGCACAAGATAAAAATATATCATCATGAACAATAAAACTGTCTGCTTACATAAAC
AGTAATACAAGGGGTGTTATGAGCCATATTCAACGGGAAACGTCGAGGCCGCGATTAAATTCCA
ACATGGATGCTGATTTATATGGGTATAAATGGGCTCGCGATAATGTCGGGCAATCAGGTGCGAC
AATCTATCGCTTGTATGGGAAGCCCGATGCGCCAGAGTTGTTTCTGAAACATGGCAAAGGTAGC
GTTGCCAATGATGTTACAGATGAGATGGTCAGACTAAACTGGCTGACGGAATTTATGCCTCTTC
CGACCATCAAGCATTTTATCCGTACTCCTGATGATGCATGGTTACTCACCACTGCGATCCCCGGA
AAAACAGCATTCCAGGTATTAGAAGAATATCCTGATTCAGGTGAAAATATTGTTGATGCGCTGG
CAGTGTTCCTGCGCCGGTTGCATTCGATTCCTGTTTGTAATTGTCCTTTTAACAGCGATCGCGTAT
TTCGTCTCGCTCAGGCGCAATCACGAATGAATAACGGTTTGGTTGATGCGAGTGATTTTG
ATGACGAGCGTAATGGCTGGCCTGTTGAACAAGTCTGGAAAGAAATGCATAAACTTTTGCCATT
CTCACCGGATTCAGTCGTCACTCATGGTGATTTCTCACTTGATAACCTTATTTTTGACGAGGGGA
AATTAATAGGTTGTATTGATGTTGGACGAGTCGGAATCGCAGACCGATACCAGGATCTTGCCAT
CCTATGGAACTGCCTCGGTGAGTTTTCTCCTTCATTACAGAAACGGCTTTTTCAAAAATATGGTA
TTGATAATCCTGATATGAATAAATTGCAGTTTCATTTGATGCTCGATGAGTTTTTCTAATCAGAA
TTGGTTAATTGGTTGTAACACTGGCAGAGCATTACGCTGACTTGACGGGACGGCGCAAGCTCAT
GACCAAAATCCCTTAACGTGAGTTACGCGTGAAGATCCTTTTTGATAATCTCATGACCAAAATC
CCTTAACGTGAGTTTTCGTTCCACTGAGCGTCAGACCCCGTAGAAAAGATCAAAGGATCTTCTT
GAGATCCTTTTTTTCTGCGCGTAATCTGCTGCTTGCAAACAAAAAAACCACCGCTACCAGCGGT
GGTTTGTTTGCCGGATCAAGAGCTACCAACTCTTTTTCCGAAGGTAACTGGCTTCAGCAGAGCG
CAGATACCAAATACTGTTCTTCTAGTGTAGCCGTAGTTAGGCCACCACTTCAAGAACTCTGTAGC
ACCGCCTACATACCTCGCTCTGCTAATCCTGTTACCAGTGGCTGCTGCCAGTGGCGATAAGTCGT
GTCTTACCGGGTTGGACTCAAGACGATAGTTACCGGATAAGGCGCAGCGGTCGGGCTGAACGG
GGGGTTCGTGCACACAGCCCAGCTTGGAGCGAACGACCTACACCGAACTGAGATACCTACAGC
GTGAGCTATGAGAAAGCGCCACGCTTCCCGAAGGGAGAAAGGCGGACAGGTATCCGGTAAGCG
GCAGGGTCGGAACAGGAGAGCGCACGAGGGAGCTTCCAGGGGGAAACGCCTGGTATCTTTATA
GTCCTGTCGGGTTTCGCCACCTCTGACTTGAGCGTCGATTTTTGTGATGCTCGTCAGGGGGGCGG
AGCCTATGGAAAAACGCCAGCAACGCGGCCTTTTTACGGTTCCTGGCCTTTTGCTGGCCTTTTGC
TCACATGT
SEQ ID NO 12: pAAV-hSYN1-hAP4B1-Kan
cctgcaggcagctgcgcgctcgctcgctcactgaggccgcccgggcaaagcccgggcgtcgggcgacctttggtcgcccg
gcctcagtgagcgagcgagcgcgcagagagggagtggccaactccatcactaggggttcctgcggccgcacgcgtccagt
gcaagtgggttttaggaccaggatgaggcggggtgggggtgcctacctgacgaccgaccccgacccactggacaagcacc
caacccccattccccaaattgcgcatcccctatcagagagggggaggggaaacaggatgcggcgaggcgcgtgcgcactg
ccagcttcagcaccgcggacagtgccttcgcccccgcctggcggcgcgcgccaccgccgcctcagcactgaaggcgcgct
gacgtcactcgccggtcccccgcaaactccccttcccggccaccttggtcgcgtccgcgccgccgccggcccagccggac
cgcaccacgcgaggcgcgagataggggggcacgggcgcgaccatctgcgctgcggcgccggcgactcagcgctgcctcag
tctgcggtgggcagcggaggagtcgtgtcgtgcctgagagcgcagggaattccccggggatcctctagagtcgacgccac
catgccgtaccttggctccgaggacgtggtgaaggagctgaagaaggctctgtgcaatcctcacattcaagctgataggc
tgcgctaccggaatgtcatccagcgagtgattaggtacatgactcaaggcttggacatgtctggtgtttttatggaaatg
gtgaaggccagtgccactgtagatattgtccagaagaagttggtttatctgtacatgtgcacatatgctcccctgaaacc
agatctggctctcctggccatcaatacgctgtgcaaagactgctcagaccccaatccaatggtgcgagggctggcgttac
ggagcatgtgtagcctcaggatgcctggtgtgcaggagtatatacaacagcctattctcaatggtctgcgggataaggct
tcatatgtcaggagagtggcagtccttggatgtgccaagatgcataatcttcatggagactctgaagtagatggtgccct
ggtaaatgaattatacagtttgctgcgtgaccaggatccaattgtagttgtgaactgcttgaggtctctagaggaaattc
tgaaacaggaaggaggcgttgtcatcaataagcccattgctcaccatctcttaaatcgaatgtcaaaactggaccaatgg
ggccaggctgaagtattgaactttctgctacgctaccaaccccgcagtgaggaagaactatttgacattctcaatctgtt
ggatagtttcctcaagagcagtagcccaggtgtggtgatgggagctaccaaactttttctgatcttggcaaaaatgtttc
cccacgtacaaactgatgtccttgtgcgggtcaagggacctttgctagctgcctgttcttcagagagccgtgagctctgt
tttgttgctctttgtcatgtacgccagatcttgcatagtttaccaggtcactttagcagccactacaaaaagtttttttg
ctcctactcggagccccactacatcaaactacagaaagtggaggtgctgtgtgaactggtgaacgatgagaatgtgcagc
aggtgctagaggagcttcgagggtactgcacggatgtgtctgcggactttgcacaggctgccatctttgccataggtggc
attgccaggacttacacagatcaatgtgttcagattttaacagagttgctgggtcttcgacaagagcacattaccacagt
ggtggtgcagactttccgagacctggtttggttgtgtcctcagtgtactgaagctgtatgtcaggccctgcccggctgtg
aagagaacattcaagatagtgaggggaagcaagcacttatttggctacttggtgtccatggggaaagaattcctaatgct
ccttatgtgttagaggactttgttgagaatgtgaagtcggaaacatttccagctgttaagatggagctgctcactgcttt
gctgcgccttttcctctcccgacctgctgagtgccaggacatgctaggacgtttgttgtattactgcatagaggaagaaa
aagatatggctgtacgggaccgaggtctcttctattatcgcctcctcttagttggcattgatgaagttaagcggattctg
tgtagccctaaatctgaccctactcttggacttttggaggatccggcagaaagacctgtgaatagctgggcctcagactt
caacacactggtgccagtgtatggcaaagcccactgggcaactatctctaaatgccagggggcagagcgttgtgacccag
agcttcctaaaacttcatcctttgccgcatcaggacccttgattcctgaagagaacaaggagagggtacaagaactccct
gattctggagccctcatgctagtccccaatcgccagcttactgctgattattttgagaaaacttggcttagccttaaagt
tgctcatcagcaagtgttgccttggcggggagaattccatcctgacaccctccagatggctcttcaagtagtgaacatcc
agaccatcgcaatgagtagggctgggtctcggccatggaaagcatacctcagtgctcaggatgatactggctgtctgttc
ttaacagaactgctattggagcctggaaactcagaaatgcagatctctgtgaaacaaaatgaagcaagaacggagacgct
gaatagttttatttctgtattagaaactgtgattggaacaattgaagaaataaaatcataagcggccgcaaagcttgcct
cgagagatctacgggtggcatccctgtgacccctccccagtgcctctcctggccctggaagttgccactccagtgcccac
cagccttgtcctaataaaattaagttgcatcattttgtctgactaggtgtccttctataatattatggggtggagggggg
tggtatggagcaaggggcaagttgggaagacaacctgtagggcctgcggggtctattgggaaccaagctggagtgcagtg
gcacaatcttggctcactgcaatctccgcctcctgggttcaagcgattctcctgcctcagcctcccgagttgttgggatt
ccaggcatgcatgaccaggctcagctaatttttgtttttttggtagagacggggtttcaccatattggccaggctggtct
ccaactcctaatctcaggtgatctacccaccttggcctcccaaattgctgggattacaggcgtgaaccactgctcccttc
cctgtccttctgattttgtaggtaaccacgtgcggaccgagcggccgcaggaacccctagtgatggagttggccactccc
tctctgcgcgctcgctcgctcactgaggccgggcgaccaaaggtcgcccgacgcccgggctttgcccgggcggcctcagt
gagcgagcgagcgcgcagctgcctgcaggGGCGCCTGATGCGGTATTTTCTCCTTACGCATCTGTGCGGTATTTC
ACACCGCATACGTCAAAGCAACCATAGTACGCGCCCTGTAGCGGCGCATTAAGCGCGGCGGGT
GTGGTGGTTACGCGCAGCGTGACCGCTACACTTGCCAGCGCCTTAGCGCCCGCTCCTTTCGCTTT
CTTCCCTTCCTTTCTCGCCACGTTCGCCGGCTTTCCCCGTCAAGCTCTAAATCGGGGGCTCCCTTT
AGGGTTCCGATTTAGTGCTTTACGGCACCTCGACCCCAAAAAACTTGATTTGGGTGATGGTTCAC
GTAGTGGGCCATCGCCCTGATAGACGGTTTTTCGCCCTTTGACGTTGGAGTCCACGTTCTTTAAT
AGTGGACTCTTGTTCCAAACTGGAACAACACTCAACTCTATCTCGGGCTATTCTTTTGATTTATA
AGGGATTTTGCCGATTTCGGTCTATTGGTTAAAAAATGAGCTGATTTAACAAAAATTTAACGCG
AATTTTAACAAAATATTAACGTTTACAATTTTATGGTGCACTCTCAGTACAATCTGCTCTGATGC
CGCATAGTTAAGCCAGCCCCGACACCCGCCAACACCCGCTGACGCGCCCTGACGGGCTTGTCTG
CTCCCGGCATCCGCTTACAGACAAGCTGTGACCGTCTCCGGGAGCTGCATGTGTCAGAGGTTTT
CACCGTCATCACCGAAACGCGCGAGACGAAAGGGCCTCGTGATACGCCTATTTTTATAGGTTAA
TGTCATGATAATAATGGTTTCTTAGACGTCCTGGCCCGTGTCTCAAAATCTCTGATGTTACATTG
CACAAGATAAAAATATATCATCATGAACAATAAAACTGTCTGCTTACATAAACAGTAATACAAG
GGGTGTTATGAGCCATATTCAACGGGAAACGTCGAGGCCGCGATTAAATTCCAACATGGATGCT
GATTTATATGGGTATAAATGGGCTCGCGATAATGTCGGGCAATCAGGTGCGACAATCTATCGCT
TGTATGGGAAGCCCGATGCGCCAGAGTTGTTTCTGAAACATGGCAAAGGTAGCGTTGCCAATGA
TGTTACAGATGAGATGGTCAGACTAAACTGGCTGACGGAATTTATGCCTCTTCCGACCATCAAG
CATTTTATCCGTACTCCTGATGATGCATGGTTACTCACCACTGCGATCCCCGGAAAAACAGCATT
CCAGGTATTAGAAGAATATCCTGATTCAGGTGAAAATATTGTTGATGCGCTGGCAGTGTTCCTG
CGCCGGTTGCATTCGATTCCTGTTTGTAATTGTCCTTTTAACAGCGATCGCGTATTTCGTCTCGCT
CAGGCGCAATCACGAATGAATAACGGTTTGGTTGATGCGAGTGATTTTGATGACGAGCGTAATG
GCTGGCCTGTT
GAACAAGTCTGGAAAGAAATGCATAAACTTTTGCCATTCTCACCGGATTCAGTCGTCACTCATG
GTGATTTCTCACTTGATAACCTTATTTTTGACGAGGGGAAATTAATAGGTTGTATTGATGTTGGA
CGAGTCGGAATCGCAGACCGATACCAGGATCTTGCCATCCTATGGAACTGCCTCGGTGAGTTTT
CTCCTTCATTACAGAAACGGCTTTTTCAAAAATATGGTATTGATAATCCTGATATGAATAAATTG
CAGTTTCATTTGATGCTCGATGAGTTTTTCTAATCAGAATTGGTTAATTGGTTGTAACACTGGCA
GAGCATTACGCTGACTTGACGGGACGGCGCAAGCTCATGACCAAAATCCCTTAACGTGAGTTAC
GCGTGAAGATCCTTTTTGATAATCTCATGACCAAAATCCCTTAACGTGAGTTTTCGTTCCACTGA
GCGTCAGACCCCGTAGAAAAGATCAAAGGATCTTCTTGAGATCCTTTTTTTCTGCGCGTAATCTG
CTGCTTGCAAACAAAAAAACCACCGCTACCAGCGGTGGTTTGTTTGCCGGATCAAGAGCTACCA
ACTCTTTTTCCGAAGGTAACTGGCTTCAGCAGAGCGCAGATACCAAATACTGTTCTTCTAGTGTA
GCCGTAGTTAGGCCACCACTTCAAGAACTCTGTAGCACCGCCTACATACCTCGCTCTGCTAATCC
TGTTACCAGTGGCTGCTGCCAGTGGCGATAAGTCGTGTCTTACCGGGTTGGACTCAAGACGATA
GTTACCGGATAAGGCGCAGCGGTCGGGCTGAACGGGGGGTTCGTGCACACAGCCCAGCTTGGA
GCGAACGACCTACACCGAACTGAGATACCTACAGCGTGAGCTATGAGAAAGCGCCACGCTTCC
CGAAGGGAGAAAGGCGGACAGGTATCCGGTAAGCGGCAGGGTCGGAACAGGAGAGCGCACGA
GGGAGCTTCCAGGGGGAAACGCCTGGTATCTTTATAGTCCTGTCGGGTTTCGCCACCTCTGACTT
GAGCGTCGATTTTTGTGATGCTCGTCAGGGGGGCGGAGCCTATGGAAAAACGCCAGCAACGCG
GCCTTTTTACGGTTCCTGGCCTTTTGCTGGCCTTTTGCTCACATGT
SEQ ID NO 13: pAAV-JeT-hAP4B1-Kan
cctgcaggcagctgcgcgctcgctcgctcactgaggccgcccgggcaaagcccgggcgtcgggcgacctttggtcgcccg
gcctcagtgagcgagcgagcgcgcagagagggagtggccaactccatcactaggggttcctgcggccgcacgcgtccggg
cggagttagggcggagccaatcagcgtgcgccgttccgaaagttgccttttatggctgggcggagaatgggcggtgaacg
ccgatgattatataaggacgcgccgggtgtggcacagctagttccgtcgcagccgggatttgggtcgcggttcttgtttg
tggaattccccggggatcctctagagtcgacgccaccatgccgtaccttggctccgaggacgtggtgaaggagctgaaga
aggctctgtgcaatcctcacattcaagctgataggctgcgctaccggaatgtcatccagcgagtgattaggtacatgact
caaggcttggacatgtctggtgtttttatggaaatggtgaaggccagtgccactgtagatattgtccagaagaagttggt
ttatctgtacatgtgcacatatgctcccctgaaaccagatctggctctcctggccatcaatacgctgtgcaaagactgct
cagaccccaatccaatggtgcgagggctggcgttacggagcatgtgtagcctcaggatgcctggtgtgcaggagtatata
caacagcctattctcaatggtctgcgggataaggcttcatatgtcaggagagtggcagtccttggatgtgccaagatgca
taatcttcatggagactctgaagtagatggtgccctggtaaatgaattatacagtttgctgcgtgaccaggatccaattg
tagttgtgaactgcttgaggtctctagaggaaattctgaaacaggaaggaggcgttgtcatcaataagcccattgctcac
catctcttaaatcgaatgtcaaaactggaccaatggggccaggctgaagtattgaactttctgctacgctaccaaccccg
cagtgaggaagaactatttgacattctcaatctgttggatagtttcctcaagagcagtagcccaggtgtggtgatgggag
ctaccaaactttttctgatcttggcaaaaatgtttccccacgtacaaactgatgtccttgtgcgggtcaagggacctttg
ctagctgcctgttcttcagagagccgtgagctctgttttgttgctctttgtcatgtacgccagatcttgcatagtttacc
aggtcactttagcagccactacaaaaagtttttttgctcctactcggagccccactacatcaaactacagaaagtggagg
tgctgtgtgaactggtgaacgatgagaatgtgcagcaggtgctagaggagcttcgagggtactgcacggatgtgtctgcg
gactttgcacaggctgccatctttgccataggtggcattgccaggacttacacagatcaatgtgttcagattttaacaga
gttgctgggtcttcgacaagagcacattaccacagtggtggtgcagactttccgagacctggtttggttgtgtcctcagt
gtactgaagctgtatgtcaggccctgcccggctgtgaagagaacattcaagatagtgaggggaagcaagcacttatttgg
ctacttggtgtccatggggaaagaattcctaatgctccttatgtgttagaggactttgttgagaatgtgaagtcggaaac
atttccagctgttaagatggagctgctcactgctttgctgcgccttttcctctcccgacctgctgagtgccaggacatgc
taggacgtttgttgtattactgcatagaggaagaaaaagatatggctgtacgggaccgaggtctcttctattatcgcctc
ctcttagttggcattgatgaagttaagcggattctgtgtagccctaaatctgaccctactcttggacttttggaggatcc
ggcagaaagacctgtgaatagctgggcctcagacttcaacacactggtgccagtgtatggcaaagcccactgggcaacta
tctctaaatgccagggggcagagcgttgtgacccagagcttcctaaaacttcatcctttgccgcatcaggacccttgatt
cctgaagagaacaaggagagggtacaagaactccctgattctggagccctcatgctagtccccaatcgccagcttactgc
tgattattttgagaaaacttggcttagccttaaagttgctcatcagcaagtgttgccttggcggggagaattccatcctg
acaccctccagatggctcttcaagtagtgaacatccagaccatcgcaatgagtagggctgggtctcggccatggaaagca
tacctcagtgctcaggatgatactggctgtctgttcttaacagaactgctattggagcctggaaactcagaaatgcagat
ctctgtgaaacaaaatgaagcaagaacggagacgctgaatagttttatttctgtattagaaactgtgattggaacaattg
aagaaataaaatcataagcggccgcaaagcttgcctcgagagatctacgggtggcatccctgtgacccctccccagtgcc
tctcctggccctggaagttgccactccagtgcccaccagccttgtcctaataaaattaagttgcatcattttgtctgact
aggtgtccttctataatattatggggtggaggggggtggtatggagcaaggggcaagttgggaagacaacctgtagggcc
tgcggggtctattgggaaccaagctggagtgcagtggcacaatcttggctcactgcaatctccgcctcctgggttcaagc
gattctcctgcctcagcctcccgagttgttgggattccaggcatgcatgaccaggctcagctaatttttgtttttttggt
agagacggggtttcaccatattggccaggctggtctccaactcctaatctcaggtgatctacccaccttggcctcccaaa
ttgctgggattacaggcgtgaaccactgctcccttccctgtccttctgattttgtaggtaaccacgtgcggaccgagcgg
ccgcaggaacccctagtgatggagttggccactccctctctgcgcgctcgctcgctcactgaggccgggcgaccaaaggt
cgcccgacgcccgggctttgcccgggcggcctcagtgagcgagcgagcgcgcagctgcctgcaggGGCGCCTGATGCGGTATTTTCT
CCTTACGCATCTGTGCGGTATTTCACACCGCATACGTCAAAGCAACCATAGTACGCGCCCTGTA
GCGGCGCATTAAGCGCGGCGGGTGTGGTGGTTACGCGCAGCGTGACCGCTACACTTGCCAGCGC
CTTAGCGCCCGCTCCTTTCGCTTTCTTCCCTTCCTTTCTCGCCACGTTCGCCGGCTTTCCCCGTCA
AGCTCTAAATCGGGGGCTCCCTTTAGGGTTCCGATTTAGTGCTTTACGGCACCTCGACCCCAAAA
AACTTGATTTGGGTGATGGTTCACGTAGTGGGCCATCGCCCTGATAGACGGTTTTTCGCCCTTTG
ACGTTGGAGTCCACGTTCTTTAATAGTGGACTCTTGTTCCAAACTGGAACAACACTCAACTCTAT
CTCGGGCTATTCTTTTGATTTATAAGGGATTTTGCCGATTTCGGTCTATTGGTTAAAAAATGAGC
TGATTTAACAAAAATTTAACGCGAATTTTAACAAAATATTAACGTTTACAATTTTATGGTGCACT
CTCAGTACAATCTGCTCTGATGCCGCATAGTTAAGCCAGCCCCGACACCCGCCAACACCCGCTG
ACGCGCCCTGACGGGCTTGTCTGCTCCCGGCATCCGCTTACAGACAAGCTGTGACCGTCTCCGG
GAGCTGCATGTGTCAGAGGTTTTCACCGTCATCACCGAAACGCGCGAGACGAAAGGGCCTCGTG
AT
ACGCCTATTTTTATAGGTTAATGTCATGATAATAATGGTTTCTTAGACGTCCTGGCCCGTGTCTC
AAAATCTCTGATGTTACATTGCACAAGATAAAAATATATCATCATGAACAATAAAACTGTCTGC
TTACATAAACAGTAATACAAGGGGTGTTATGAGCCATATTCAACGGGAAACGTCGAGGCCGCG
ATTAAATTCCAACATGGATGCTGATTTATATGGGTATAAATGGGCTCGCGATAATGTCGGGCAA
TCAGGTGCGACAATCTATCGCTTGTATGGGAAGCCCGATGCGCCAGAGTTGTTTCTGAAACATG
GCAAAGGTAGCGTTGCCAATGATGTTACAGATGAGATGGTCAGACTAAACTGGCTGACGGAATT
TATGCCTCTTCCGACCATCAAGCATTTTATCCGTACTCCTGATGATGCATGGTTACTCACCACTG
CGATCCCCGGAAAAACAGCATTCCAGGTATTAGAAGAATATCCTGATTCAGGTGAAAATATTGT
TGATGCGCTGGCAGTGTTCCTGCGCCGGTTGCATTCGATTCCTGTTTGTAATTGTCCTTTTAACA
GCGATCGCGTATTTCGTCTCGCTCAGGCGCAATCACGAATGAATAACGGTTTGGTTGATGCGAG
TGATTTTGATGACGAGCGTAATGGCTGGCCTGTTGAACAAGTCTGGAAAGAAATGCATAAACTT
TTGCCATTCTCACCGGATTCAGTCGTCACTCATGGTGATTTCTCACTTGATAACCTTATTTTTGAC
GAGGGGAAATTAATAGGTTGTATTGATGTTGGACGAGTCGGAATCGCAGACCGATACCAGGAT
CTTGCCATCCTATGGAACTGCCTCGGTGAGTTTTCTCCTTCATTACAGAAACGGCTTTTTCAAAA
ATATGGTATTGATAATCCTGATATGAATAAATTGCAGTTTCATTTGATGCTCGATGAGTTTTTCT
AATCAGAATTGGTTAATTGGTTGTAACACTGGCAGAGCATTACGCTGACTTGACGGGACGGCGC
AAGCTCATGACCAAAATCCCTTAACGTGAGTTACGCGTGAAGATCCTTTTTGATAATCTCATGAC
CAAAATCCCTTAACGTGAGTTTTCGTTCCACTGAGCGTCAGACCCCGTAGAAAAGATCAAAGGA
TCTTCTTGAGATCCTTTTTTTCTGCGCGTAATCTGCTGCTTGCAAACAAAAAAACCACCGCTACC
AGCGGTGGTTTGTTTGCCGGATCAAGAGCTACCAACTCTTTTTCCGAAGGTAACTGGCTTCAGCA
GAGCGCAGATACCAAATACTGTTCTTCTAGTGTAGCCGTAGTTAGGCCACCACTTCAAGAACTC
TGTAGCAC
CGCCTACATACCTCGCTCTGCTAATCCTGTTACCAGTGGCTGCTGCCAGTGGCGATAAGTCGTGT
CTTACCGGGTTGGACTCAAGACGATAGTTACCGGATAAGGCGCAGCGGTCGGGCTGAACGGGG
GGTTCGTGCACACAGCCCAGCTTGGAGCGAACGACCTACACCGAACTGAGATACCTACAGCGT
GAGCTATGAGAAAGCGCCACGCTTCCCGAAGGGAGAAAGGCGGACAGGTATCCGGTAAGCGGC
AGGGTCGGAACAGGAGAGCGCACGAGGGAGCTTCCAGGGGGAAACGCCTGGTATCTTTATAGT
CCTGTCGGGTTTCGCCACCTCTGACTTGAGCGTCGATTTTTGTGATGCTCGTCAGGGGGGCGGAG
CCTATGGAAAAACGCCAGCAACGCGGCCTTTTTACGGTTCCTGGCCTTTTGCTGGCCTTTTGCTC
ACATGT
SEQ ID NO 14: pAAV-MeP229-hAP4B1-Kan
cctgcaggcagctgcgcgctcgctcgctcactgaggccgcccgggcaaagcccgggcgtcgggcgacctttggtcgcccggcctcag
tgagcgagcgagcgcgcagagagggagtggccaactccatcactaggggttcctgcggccgcacgcgtccCAATTGAGGGCGTCACC
GCTAAGGCTCCGCCCCAGCCTGGGCTCCACAACCAATGAAGGGTAATCTCGACAAAGAGCAAGGGGTGGGGC
GCGGGCGCGCAGGTGCAGCAGCACACAGGCTGGTCGGGAGGGCGGGGCGCGACGTCTGCCGTG
CGGGGTCCCGGCATCGGTTGCGCGCGCGCTCCCTCCTCTCGGAGAGAGGGCTGTGGTAAAACCC
GTCCGGAAAggaattccccggggatcctctagagtcgacgccaccatgccgtaccttggctccgaggacgtggtgaaggagctgaag
aaggctctgtgcaatcctcacattcaagctgataggctgcgctaccggaatgtcatccagcgagtgattaggtacatgactcaaggc
ttggacatgtctggtgtttttatggaaatggtgaaggccagtgccactgtagatattgtccagaagaagttggtttatctgtacatg
tgcacatatgctcccctgaaaccagatctggctctcctggccatcaatacgctgtgcaaagactgctcagaccccaatccaatggt
gcgagggctggcgttacggagcatgtgtagcctcaggatgcctggtgtgcaggagtatatacaacagcctattctcaatg
gtctgcgggataaggcttcatatgtcaggagagtggcagtccttggatgtgccaagatgcataatcttcatggagactct
gaagtagatggtgccctggtaaatgaattatacagtttgctgcgtgaccaggatccaattgtagttgtgaactgcttgag
gtctctagaggaaattctgaaacaggaaggaggcgttgtcatcaataagcccattgctcaccatctcttaaatcgaatgt
caaaactggaccaatggggccaggctgaagtattgaactttctgctacgctaccaaccccgcagtgaggaagaactattt
gacattctcaatctgttggatagtttcctcaagagcagtagcccaggtgtggtgatgggagctaccaaactttttctgat
cttggcaaaaatgtttccccacgtacaaactgatgtccttgtgcgggtcaagggacctttgctagctgcctgttcttcag
agagccgtgagctctgttttgttgctctttgtcatgtacgccagatcttgcatagtttaccaggtcactttagcagccac
tacaaaaagtttttttgctcctactcggagccccactacatcaaactacagaaagtggaggtgctgtgtgaactggtgaa
cgatgagaatgtgcagcaggtgctagaggagcttcgagggtactgcacggatgtgtctgcggactttgcacaggctgcca
tctttgccataggtggcattgccaggacttacacagatcaatgtgttcagattttaacagagttgctgggtcttcgacaa
gagcacattaccacagtggtggtgcagactttccgagacctggtttggttgtgtcctcagtgtactgaagctgtatgtca
ggccctgcccggctgtgaagagaacattcaagatagtgaggggaagcaagcacttatttggctacttggtgtccatgggg
aaagaattcctaatgctccttatgtgttagaggactttgttgagaatgtgaagtcggaaacatttccagctgttaagatg
gagctgctcactgctttgctgcgccttttcctctcccgacctgctgagtgccaggacatgctaggacgtttgttgtatta
ctgcatagaggaagaaaaagatatggctgtacgggaccgaggtctcttctattatcgcctcctcttagttggcattgatg
aagttaagcggattctgtgtagccctaaatctgaccctactcttggacttttggaggatccggcagaaagacctgtgaat
agctgggcctcagacttcaacacactggtgccagtgtatggcaaagcccactgggcaactatctctaaatgccagggggc
agagcgttgtgacccagagcttcctaaaacttcatcctttgccgcatcaggacccttgattcctgaagagaacaaggaga
gggtacaagaactccctgattctggagccctcatgctagtccccaatcgccagcttactgctgattattttgagaaaact
tggcttagccttaaagttgctcatcagcaagtgttgccttggcggggagaattccatcctgacaccctccagatggctct
tcaagtagtgaacatccagaccatcgcaatgagtagggctgggtctcggccatggaaagcatacctcagtgctcaggatg
atactggctgtctgttcttaacagaactgctattggagcctggaaactcagaaatgcagatctctgtgaaacaaaatgaa
gcaagaacggagacgctgaatagttttatttctgtattagaaactgtgattggaacaattgaagaaataaaatcataagc
ggccgcaaagcttgcctcgagagatctacgggtggcatccctgtgacccctccccagtgcctctcctggccctggaagtt
gccactccagtgcccaccagccttgtcctaataaaattaagttgcatcattttgtctgactaggtgtccttctataatat
tatggggtggaggggggtggtatggagcaaggggcaagttgggaagacaacctgtagggcctgcggggtctattgggaac
caagctggagtgcagtggcacaatcttggctcactgcaatctccgcctcctgggttcaagcgattctcctgcctcagcct
cccgagttgttgggattccaggcatgcatgaccaggctcagctaatttttgtttttttggtagagacggggtttcaccat
attggccaggctggtctccaactcctaatctcaggtgatctacccaccttggcctcccaaattgctgggattacaggcgt
gaaccactgctcccttccctgtccttctgattttgtaggtaaccacgtgcggaccgagcggccgcaggaacccctagtga
tggagttggccactccctctctgcgcgctcgctcgctcactgaggccgggcgaccaaaggtcgcccgacgcccgggcttt
gcccgggcggcctcagtgagcgagcgagcgcgcagctgcctgcaggGGCGCCTGATGCGGTATTTTCTCCTTACGCATCT
GTGCGGTATTTCACACCGCATACGTCAAAGCAACCATAGTACGCGCCCTGTAGCGGCGCATTAA
GCGCGGCGGGTGTGGTGGTTACGCGCAGCGTGACCGCTACACTTGCCAGCGCCTTAGCGCCCGC
TCCTTTCGCTTTCTTCCCTTCCTTTCTCGCCACGTTCGCCGGCTTTCCCCGTCAAGCTCTAAATCG
GGGGCTCCCTTTAGGGTTCCGATTTAGTGCTTTACGGCACCTCGACCCCAAAAAACTTGATTTGG
GTGATGGTTCACGTAGTGGGCCATCGCCCTGATAGACGGTTTTTCGCCCTTTGACGTTGGAGTCC
ACGTTCTTTAATAGTGGACTCTTGTTCCAAACTGGAACAACACTCAACTCTATCTCGGGCTATTC
TTTTGATTTATAAGGGATTTTGCCGATTTCGGTCTATTGGTTAAAAAATGAGCTGATTTAACAAA
AATTTAACGCGAATTTTAACAAAATATTAACGTTTACAATTTTATGGTGCACTCTCAGTACAATC
TGCTCTGATGCCGCATAGTTAAGCCAGCCCCGACACCCGCCAACACCCGCTGACGCGCCCTGAC
GGGCTTGTCTGCTCCCGGCATCCGCTTACAGACAAGCTGTGACCGTCTCCGGGAGCTGCATGTG
TCAGAGGTTTTCACCGTCATCACCGAAACGCGCGAGACGAAAGGGCCTCGTGATACGCCTATTT
TTATAGGTTAATGTCATGATAATAATGGTTTCTTAGACGTCCTGGCCCGTGTCTCAAAATCTCTG
ATGTTACATTGCACAAGATAAAAATATATCATCATGAACAATAAAACTGTCTGCTTACATAAAC
AGTAATACAAGGGGTGTTATGAGCCATATTCAACGGGAAACGTCGAGGCCGCGATTAAATTCCA
ACATGGATGCTGATTTATATGGGTATAAATGGGCTCGCGATAATGTCGGGCAATCAGGTGCGAC
AATCTATCGCTTGTATGGGAAGCCCGATGCGCCAGAGTTGTTTCTGAAACATGGCAAAGGTAGC
GTTGCCAATGATGTTACAGATGAGATGGTCAGACTAAACTGGCTGACGGAATTTATGCCTCTTC
CGACCATCAAGCATTTTATCCGTACTCCTGATGATGCATGGTTACTCACCACTGCGATCCCCGGA
AAAACAGCATTCCAGGTATTAGAAGAATATCCTGATTCAGGTGAAAATATTGTTGATGCGCTGG
CAGTGTTCCTGCGCCGGTTGCATTCGATTCCTGTTTGTAATTGTCCTTTTAACAGCGATCGCGTAT
TTCGTCTCGCTCAGGCGCAATCACGAATGAATAACGGTTTGGTTGATGCGAGTGATTTTGATGA
CGAGC
GTAATGGCTGGCCTGTTGAACAAGTCTGGAAAGAAATGCATAAACTTTTGCCATTCTCACCGGA
TTCAGTCGTCACTCATGGTGATTTCTCACTTGATAACCTTATTTTTGACGAGGGGAAATTAATAG
GTTGTATTGATGTTGGACGAGTCGGAATCGCAGACCGATACCAGGATCTTGCCATCCTATGGAA
CTGCCTCGGTGAGTTTTCTCCTTCATTACAGAAACGGCTTTTTCAAAAATATGGTATTGATAATC
CTGATATGAATAAATTGCAGTTTCATTTGATGCTCGATGAGTTTTTCTAATCAGAATTGGTTAAT
TGGTTGTAACACTGGCAGAGCATTACGCTGACTTGACGGGACGGCGCAAGCTCATGACCAAAAT
CCCTTAACGTGAGTTACGCGTGAAGATCCTTTTTGATAATCTCATGACCAAAATCCCTTAACGTG
AGTTTTCGTTCCACTGAGCGTCAGACCCCGTAGAAAAGATCAAAGGATCTTCTTGAGATCCTTTT
TTTCTGCGCGTAATCTGCTGCTTGCAAACAAAAAAACCACCGCTACCAGCGGTGGTTTGTTTGCC
GGATCAAGAGCTACCAACTCTTTTTCCGAAGGTAACTGGCTTCAGCAGAGCGCAGATA
CCAAATACTGTTCTTCTAGTGTAGCCGTAGTTAGGCCACCACTTCAAGAACTCTGTAGCACCGCC
TACATACCTCGCTCTGCTAATCCTGTTACCAGTGGCTGCTGCCAGTGGCGATAAGTCGTGTCTTA
CCGGGTTGGACTCAAGACGATAGTTACCGGATAAGGCGCAGCGGTCGGGCTGAACGGGGGGTT
CGTGCACACAGCCCAGCTTGGAGCGAACGACCTACACCGAACTGAGATACCTACAGCGTGAGC
TATGAGAAAGCGCCACGCTTCCCGAAGGGAGAAAGGCGGACAGGTATCCGGTAAGCGGCAGGG
TCGGAACAGGAGAGCGCACGAGGGAGCTTCCAGGGGGAAACGCCTGGTATCTTTATAGTCCTGT
CGGGTTTCGCCACCTCTGACTTGAGCGTCGATTTTTGTGATGCTCGTCAGGGGGGCGGAGCCTAT
GGAAAAACGCCAGCAACGCGGCCTTTTTACGGTTCCTGGCCTTTTGCTGGCCTTTTGCTCACATG
T
SEQ ID NO 15 AP4B1

| | |
|---|---|
| atgccgtacc ttggctccga ggacgtggtg aaggagctga agaaggctct gtgcaatcct | 60 |
| cacattcaag ctgataggct gcgctaccgg aatgtcatcc agcgagtgat taggtacatg | 120 |
| actcaaggct tggacatgtc tggtgttttt atggaaatgg tgaaggccag tgccactgta | 180 |
| gatattgtcc agaagaagtt ggtttatctg tacatgtgca catatgctcc cctgaaacca | 240 |
| gatctggctc tcctggccat caatacgctg tgcaaagact gctcagaccc caatccaatg | 300 |
| gtgcgagggc tggcgttacg gagcatgtgt agcctcagga tgcctggtgt gcaggagtat | 360 |
| atacaacagc ctattctcaa tggtctgcgg gataaggctt catatgtcag gagagtggca | 420 |
| gtccttggat gtgccaagat gcataatctt catggagact ctgaagtaga tggtgccctg | 480 |
| gtaaatgaat tatacagttt gctgcgtgac caggatccaa ttgtagttgt gaactgcttg | 540 |
| aggtctctag aggaaattct gaaacaggaa ggaggcgttg tcatcaataa gcccattgct | 600 |
| caccatctct taaatcgaat gtcaaaactg gaccaatggg gccaggctga agtattgaac | 660 |
| tttctgctac gctaccaacc ccgcagtgag gaagaactat ttgacattct caatctgttg | 720 |
| gatagtttcc tcaagagcag tagcccaggt gtggtgatgg gagctaccaa actttttctg | 780 |
| atcttggcaa aaatgtttcc ccacgtacaa actgatgtcc ttgtgcgggt caagggacct | 840 |
| ttgctagctg cctgttcttc agagagccgt gagctctgtt ttgttgctct ttgtcatgta | 900 |
| cgccagatct tgcatagttt accaggtcac tttagcagcc actacaaaaa gtttttttgc | 960 |
| tcctactcgg agccccacta catcaaacta cagaaagtgg aggtgctgtg tgaactggtg | 1020 |
| aacgatgaga atgtgcagca ggtgctagag gagcttcgag ggtactgcac ggatgtgtct | 1080 |
| gcggactttg cacaggctgc catctttgcc ataggtggca ttgccaggac ttacacagat | 1140 |
| caatgtgttc agattttaac agagttgctg ggtcttcgac aagagcacat taccacagtg | 1200 |
| gtggtgcaga ctttccgaga cctggtttgg ttgtgtcctc agtgtactga agctgtatgt | 1260 |
| caggccctgc ccggctgtga agagaacatt caagatagtg aggggaagca agcacttatt | 1320 |
| tggctacttg gtgtccatgg ggaaagaatt cctaatgctc cttatgtgtt agaggacttt | 1380 |
| gttgagaatg tgaagtcgga aacatttcca gctgttaaga tggagctgct cactgctttg | 1440 |
| ctgcgccttt tcctctcccg acctgctgag tgccaggaca tgctaggacg tttgttgtat | 1500 |
| tactgcatag aggaagaaaa agatatggct gtacgggacc gaggtctctt ctattatcgc | 1560 |
| ctcctcttag ttggcattga tgaagttaag cggattctgt gtagccctaa atctgaccct | 1620 |
| actcttggac ttttggagga tccggcagaa agacctgtga atagctgggc ctcagacttc | 1680 |
| aacacactgg tgccagtgta tggcaaagcc cactgggcaa ctatctctaa atgccagggg | 1740 |
| gcagagcgtt gtgacccaga gcttcctaaa acttcatcct ttgccgcatc aggacccttg | 1800 |
| attcctgaag agaacaagga gagggtacaa gaactccctg attctggagc cctcatgcta | 1860 |
| gtccccaatc gccagcttac tgctgattat tttgagaaaa cttggcttag ccttaaagtt | 1920 |
| gctcatcagc aagtgttgcc ttggcgggga gaattccatc ctgacaccct ccagatggct | 1980 |
| cttcaagtag tgaacatcca gaccatcgca atgagtaggg ctgggtctcg gccatggaaa | 2040 |
| gcatacctca gtgctcagga tgatactggc tgtctgttct taacagaact gctattggag | 2100 |
| cctggaaact cagaaatgca gatctctgtg aaacaaaatg aagcaagaac ggagacgctg | 2160 |
| aatagtttta tttctgtatt agaaactgtg attggaacaa ttgaagaaat aaaatcataa | 2220 |

SEQ ID NO 16 AP4B1 PRT

| | |
|---|---|
| MPYLGSEDVV KELKKALCNP HIQADRLRYR NVIQRVIRYM TQGLDMSGVF MEMVKASATV | 60 |
| DIVQKKLVYL YMCTYAPLKP DLALLAINTL CKDCSDPNPM VRGLALRSMC SLRMPGVQEY | 120 |
| IQQPILNGLR DKASYVRRVA VLGCAKMHNL HGDSEVDGAL VNELYSLLRD QDPIVVVNCL | 180 |
| RSLEEILKQE GGVVINKPIA HHLLNRMSKL DQWGQAEVLN FLLRYQPRSE EELFDILNLL | 240 |
| DSFLKSSSPG VVMGATKLFL ILAKMFPHVQ TDVLVRVKGP LLAACSSESR ELCFVALCHV | 300 |
| RQILHSLPGH FSSHYKKFFC SYSEPHYIKL QKVEVLCELV NDENVQQVLE ELRGYCTDVS | 360 |
| ADFAQAAIFA IGGIARTYTD QCVQILTELL GLRQEHITTV VVQTFRDLVW LCPQCTEAVC | 420 |
| QALPGCEENI QDSEGKQALI WLLGVHGERI PNAPYVLEDF VENVKSETFP AVKMELLTAL | 480 |
| LRLFLSRPAE CQDMLGRLLY YCIEEEKDMA VRDRGLFYYR LLLVGIDEVK RILCSPKSDP | 540 |
| TLGLLEDPAE RPVNSWASDF NTLVPVYGKA HWATISKCQG AERCDPELPK TSSFAASGPL | 600 |
| IPEENKERVQ ELPDSGALML VPNRQLTADY FEKTWLSLKV AHQQVLPWRG EFHPDTLQMA | 660 |
| LQVVNIQTIA MSRAGSRPWK AYLSAQDDTG CLFLTELLLE PGNSEMQISV KQNEARTETL | 720 |
| NSFISVLETV IGTIEEIKS | 739 |

SEQ ID NO 17 AP4E1

| | |
|---|---|
| atgagcgaca tagtggagaa gacgctgacg gcgctgccgg gactctttct gcagaaccag | 60 |
| cccggtggtg ggcccgcggc cgccaaggcg tccttctcct cgaggctggg cagccttgtc | 120 |
| cgcggcatca cagccctcac ctccaagcac gaagaagaaa aattaatcca gcaggaactg | 180 |
| agtagtctga aagcgactgt ttctgctcct actacaacac tgaaaatgat gaaggaatgt | 240 |
| atggtgagac ttatatattg tgaaatgctt ggatatgatg cttcctttgg ctatattcat | 300 |
| gcaatcaagt tagcccaaca aggaaacctc ttagaaaaaa gagtaggtta tttggctgtt | 360 |
| tccttatttc tacatgaaag tcatgaatta ttgcttctcc ttgtgaatac agttgtaaag | 420 |
| gatctgcaga gcactaacct agtagaagtg tgtatggcac tgactgttgt tagccagatt | 480 |
| ttcccctgcg aaatgattcc agctgttctt ccattaatag aagataaact tcaacattct | 540 |
| aaggagattg tacgaagaaa agctgttctg gcattataca aattccatct cattgctcct | 600 |
| aatcaagtac aacatattca tattaagttt cggaaagcac tttgtgacag agatgttggg | 660 |
| gtcatggctg cctccttgca tatatatctt agaatgatta aggagaattc atctggatat | 720 |
| aaagacttga ctgggagttt tgtaaccatt ttgaagcaag tagttggagg aaagctccca | 780 |
| gtagaattca attaccacag tgtgccagca ccatggttac aaattcagct cttgagaata | 840 |
| ctgggacttc taggaaaaga tgatcaaagg acaagtgaat taatgtatga tgttcttgat | 900 |
| gaatccttac gaagagctga gttaaatcac aatgtcacat atgctatttt gtttgaatgt | 960 |
| gtgcatacag tctattctat ttatcctaaa tcggaattac ttgagaaggc tgccaagtgc | 1020 |
| attggaaaat ttgttctgtc acctaaaata aatctaaaat atttaggact gaaggctctt | 1080 |
| acctatgtta tccaacagga tcctactctg gctcttcaac accagatgac aataattgaa | 1140 |
| tgtttagatc atcctgatcc cattattaaa agagagactc tggaacttct ttacagaatt | 1200 |
| actaatgcac agaatataac agttattgtc cagaaaatgc ttgaatattt acatcagagc | 1260 |
| aaagaagagt atgtcatcgt caatttggtc ggcaaaatag cagagctggc tgagaaatat | 1320 |
| gctcctgata atgcatggtt tattcagaca atgaatgctg tgttttcagt aggaggagat | 1380 |
| gtaatgcatc ctgatattcc caataacttt ctgagactac tagcggaagg ttttgatgat | 1440 |
| gaaacagaag atcagcaatt aagactctat gcagttcagt cttatctcac tttactggat | 1500 |
| atggaaaatg tgttctatcc acagagattt cttcaagtta tgagttgggt attaggggaa | 1560 |
| tattcctacc tcttagataa ggaaacgcca gaggaagtta tagctaagct ctacaagtta | 1620 |
| cttatgaatg actctgtgtc ttcagaaaca aaagcctggt taattgctgc tgtgaccaaa | 1680 |
| ttgacatctc aggcgcactc ttctaataca gttgagagat taatccatga atttaccata | 1740 |
| tctttggata cttgtatgag acaacatgca tttgaattaa aacatttgca tgagaatgtg | 1800 |
| gaacttatga agagcttgct tccagttgac aggagttgtg aagacttggt ggtagatgct | 1860 |
| tctttatctt ttctggatgg ttttgtggct gaaggactca gtcagggtgc agcgccttac | 1920 |
| aaacctcccc atcaacgcca ggaggaaaag ctttctcagg aaaaagttct caattttgaa | 1980 |
| ccatatggac tctccttttc ttcatctggc ttcactggac gacagtctcc tgctggcatt | 2040 |
| tctcttggtt cagatgtatc tgggaatagt gctgagacag gactgaaaga gacaaatagc | 2100 |
| ttgaagctgg aaggtataaa gaaattgtgg gggaaagaag gctatcttcc caagaaggaa | 2160 |
| agcaaaactg gtgatgaaag tggagctctg cctgttcctc aagagagtat aatggagaat | 2220 |
| gtagatcaag ctataactaa aaaggatcaa tctcaagttc ttacccaatc taaagaggag | 2280 |
| aaagaaaagc agctgctggc atcatcatta tttgttggtc taggatcaga aagtacaatc | 2340 |
| aacctgctgg gaaaagcaga tactgtctct cacaagttca gaaggaaatc aaaagtcaaa | 2400 |
| gaagctaaaa gtggcgaaac aaccagtact cataatatga cctgttcttc ctttagttct | 2460 |
| ttgtcaaatg tggcatatga agatgattat tattcgaata ctttgcacga tacaggagac | 2520 |
| aaggaattaa agaaattttc tctcacttca gaacttttgg attctgagtc actcacagaa | 2580 |
| ctgcccttgg ttgagaaatt ctcatattgt agtctgtcta caccttcatt gtttgctaat | 2640 |
| aacaacatgg aaatttttca ccctcctcaa tctactgcag cctcagttgc caaggaaagc | 2700 |
| tctttagctt catctttttt ggaagaaact actgaataca tacactcaaa tgctatggaa | 2760 |
| gtctgtaata atgaaactat atcagtgtct tcttataaaa tttggaaaga tgattgttta | 2820 |
| ttgatggtct ggtcagtcac taataagagt ggtttggaat tgaaaagtgc tgacttagaa | 2880 |
| atttttcctg cagaaaattt caaggtgact gagcaacctg gatgctgttt gcctgtaatg | 2940 |
| gaagcagaaa gcaccaaaag ctttcaatat agtgtgcaga tagaaaaacc ttttacagaa | 3000 |
| ggaaatctta ctggttttat tagttatcat atgatggata ctcattctgc tcagctggaa | 3060 |
| ttttctgtaa acttatcact attagatttc attagaccat taaaaatctc aagtgacgac | 3120 |
| tttgggaaac tctggttatc cttcgcaaat gatgtgaaac aaaatgtaaa aatgtcagaa | 3180 |
| tctcaagctg cacttccttc tgcactaaag actctgcaac agaaactaag actccatatt | 3240 |
| attgagatta taggcaatga agggctattg gcctgtcagc tgctcccatc catcccctgc | 3300 |
| ttactgcatt gccgagttca tgcagatgta ttagccctgt ggttcagatc ctcctgttct | 3360 |
| actcttcctg actatttact gtatcagtgt caaaaggtga tggagggatc ctag | 3414 |
| SEQ ID NO 18 AP4E1 PRT | |
| MSDIVEKTLT ALPGLFLQNQ PGGGPAAAKA SFSSRLGSLV RGITALTSKH EEEKLIQQEL | 60 |
| SSLKATVSAP TTTLKMMKEC MVRLIYCEML GYDASFGYIH AIKLAQQGNL LEKRVGYLAV | 120 |
| SLFLHESHEL LLLLVNTVVK DLQSTNLVEV CMALTVVSQI FPCEMIPAVL PLIEDKLQHS | 180 |
| KEIVRRKAVL ALYKFHLIAP NQVQHIHIKF RKALCDRDVG VMAASLHIYL RMIKENSSGY | 240 |
| KDLTGSFVTI LKQVVGGKLP VEFNYHSVPA PWLQIQLLRI LGLLGKDDQR TSELMYDVLD | 300 |
| ESLRRAELNH NVTYAILFEC VHTVYSIYPK SELLEKAAKC IGKFVLSPKI NLKYLGLKAL | 360 |
| TYVIQQDPTL ALQHQMTIIE CLDHPDPIIK RETLELLYRI TNAQNITVIV QKMLEYLHQS | 420 |
| KEEYVIVNLV GKIAELAEKY APDNAWFIQT MNAVFSVGGD VMHPDIPNNF LRLLAEGFDD | 480 |
| ETEDQQLRLY AVQSYLTLLD MENVFYPQRF LQVMSWVLGE YSYLLDKETP EEVIAKLYKL | 540 |
| LMNDSVSSET KAWLIAAVTK LTSQAHSSNT VERLIHEFTI SLDTCMRQHA FELKHLHENV | 600 |
| ELMKSLLPVD RSCEDLVVDA SLSFLDGFVA EGLSQGAAPY KPPHQRQEEK LSQEKVLNFE | 660 |
| PYGLSFSSSG FTGRQSPAGI SLGSDVSGNS AETGLKETNS LKLEGIKKLW GKEGYLPKKE | 720 |
| SKTGDESGAL PVPQESIMEN VDQAITKKDQ SQVLTQSKEE KEKQLLASSL FVGLGSESTI | 780 |
| NLLGKADTVS HKFRRKSKVK EAKSGETTST HNMTCSSFSS LSNVAYEDDY YSNTLHDTGD | 840 |
| KELKKFSLTS ELLDSESLTE LPLVEKFSYC SLSTPSLFAN NNMEIFHPPQ STAASVAKES | 900 |
| SLASSFLEET TEYIHSNAME VCNNETISVS SYKIWKDDCL LMVWSVTNKS GLELKSADLE | 960 |
| IFPAENFKVT EQPGCCLPVM EAESTKSFQY SVQIEKPFTE GNLTGFISYH MMDTHSAQLE | 1020 |
| FSVNLSLLDF IRPLKISSDD FGKLWLSFAN DVKQNVKMSE SQAALPSALK TLQQKLRLHI | 1080 |
| IEIIGNEGLL ACQLLPSIPC LLHCRVHADV LALWERSSCS TLPDYLLYQC QKVMEGS | 1137 |

SEQ ID NO 19 AP4M1

| | |
|---|---|
| atgatttccc aattcttcat tctgtcctcc aagggggacc cgctcatcta caaagacttc | 60 |
| cgcggggaca gtggcggccg ggatgtggcc gagctcttct accggaagct gacgggactg | 120 |
| ccaggagacg agtccccggt tgtcatgcat caccatggcc gtcatttcat tcacatcaga | 180 |
| cacagcggcc tctatttggt ggtcacaact tcagaaaacg tttctccctt cagcctccta | 240 |
| gaactgctct ccaggttggc cacccttctg ggcgattact gtggctccct gggcgagggg | 300 |
| accatctccc gcaatgtggc tctggtatac gaactcctgg atgaagtgct ggactatggc | 360 |
| tatgtacaga ccacatccac ggagatgctg aggaatttca tccagacgga agctgtggtc | 420 |
| agcaagccct tcagcctctt tgacctcagc agcgttggct tgtttggggc tgagacacaa | 480 |
| cagagcaaag tggcccccag cagtgcagcc agccgccccg tcctgtccag tcgctctgac | 540 |
| cagagccaaa agaatgaagt ttttttggat gtggtcgaga gattgtctgt actgatagca | 600 |
| tctaatggat ccctgctgaa ggtggatgtg cagggagaga ttcggctcaa gagcttcctt | 660 |
| cctagcggct ctgagatgcg cattggcttg acggaagagt tttgtgtggg gaagtcagag | 720 |
| ctgagaggtt atgggccagg aatccgggtc gatgaagtct cgtttcacag ctctgtgaat | 780 |
| ctggacgaat ttgagtctca tcgaatcctc cgcttgcaac cacctcaggg cgagctgact | 840 |
| gtgatgcggt accaactctc cgatgacctc ccctcaccgc tccccttccg gctcttcccc | 900 |
| tctgtgcagt gggaccgagg ctcaggccgg ctccaggttt atctaaagtt gcgatgtgac | 960 |
| ctgctctcaa agagccaagc cctcaatgtc aggctgcacc tccccctgcc tcgaggggtg | 1020 |
| gtcagcctgt ctcaggagct gagcagccca gagcagaagg ctgagctggc agagggagcc | 1080 |
| cttcgctggg acctgcctcg ggtgcaagga ggctctcaac tctcaggcct tttccagatg | 1140 |
| gacgtcccag ggcccccagg acctcccagc catgggctct ccacctcggc ctctcctctg | 1200 |
| gggctgggcc ctgccagtct ctccttcgag cttccccggc acacgtgctc tggcctccag | 1260 |
| gtccgattcc tcaggctggc cttcaggcca tgcggcaatg ccaaccccca caagtgggtg | 1320 |
| cgacacctaa gccacagcga cgcctatgtc attcggatct ga | 1362 |

SEQ ID NO 20 AP4M1 PRT

| | |
|---|---|
| MISQFFILSS KGDPLIYKDF RGDSGGRDVA ELFYRKLTGL PGDESPVVMH HHGRHFIHIR | 60 |
| HSGLYLVVTT SENVSPFSLL ELLSRLATLL GDYCGSLGEG TISRNVALVY ELLDEVLDYG | 120 |
| YVQTTSTEML RNFIQTEAVV SKPFSLFDLS SVGLFGAETQ QSKVAPSSAA SRPVLSSRSD | 180 |
| QSQKNEVELD VVERLSVLIA SNGSLLKVDV QGEIRLKSFL PSGSEMRIGL TEEFCVGKSE | 240 |
| LRGYGPGIRV DEVSFHSSVN LDEFESHRIL RLQPPQGELT VMRYQLSDDL PSPLPFRLFP | 300 |
| SVQWDRGSGR LQVYLKLRCD LLSKSQALNV RLHLPLPRGV VSLSQELSSP EQKAELAEGA | 360 |
| LRWDLPRVQG GSQLSGLFQM DVPGPPGPPS HGLSTSASPL GLGPASLSFE LPRHTCSGLQ | 420 |
| VRFLRLAFRP CGNANPHKWV RHLSHSDAYV IRI | 453 |

SEQ ID NO 21 AP4S1

| | |
|---|---|
| atgataaaat ttttcctcat ggtgaataaa caagggcaga ctcgactttc taagtactat | 60 |
| gaacatgtgg atattaataa gcgtacactt ctggaaacag aagtcataaa gagctgtctc | 120 |
| tctcgatcca atgaacaatg ctctttcatt gaatataagg attttaagct gatatatcgg | 180 |
| cagtatgcag ctctcttcat tgtggttgga gttaatgaca ctgagaacga gatggctatt | 240 |
| tatgaattca ttcataactt tgtggaagtt ttagatgagt atttcagccg agtgagtgaa | 300 |
| ttagatgtat cctttttcaa tactgttttc cacagtactt ggcaaatgca ctctggtcct | 360 |
| tatcaggaac caattgatga acttcccaaa atatgctcag ccctggagcc ccagcagact | 420 |
| tgcttttctc cagacagttc atcattcaaa ggagctgcct ccaccacccc catctactga | 480 |

SEQ ID NO 22 AP4S1 PRT

| | |
|---|---|
| MIKFFLMVNK QGQTRLSKYY EHVDINKRTL LETEVIKSCL SRSNEQCSFI EYKDFKLIYR | 60 |
| QYAALFIVVG VNDTENEMAI YEFIHNFVEV LDEYFSRVSE LDVSFFNTVF HSTWQMHSGP | 120 |
| YQEPIDELPK ICSALEPQQT CFSPDSSSFK GAASTTPIY | 159 |

SEQ ID NO 23 AAV-CBh-hAP4B1-hGH polyA-Kan
CCTGCAGGCAGCTGCGCGCTCGCTCGCTCACTGAGGCCGCCCGGGCAAAGCCCGGGCGTCGGGCGACCTTTGGTCGCCCG
GCCTCAGTGAGCGAGCGAGCGCGCAGAGAGGGAGTGGCCAACTCCATCACTAGGGGTTCCTGCGGCCGCACGCGTCCCGT
TACATAACTTACGGTAAATGGCCCGCCTGGCTGACCGCCCAACGACCCCCGCCCATTGACGTCAATAGTAACGCCAATAG
GGACTTTCCATTGACGTCAATGGGTGGAGTATTTACGGTAAACTGCCCACTTGGCAGTACATCAAGTGTATCATATGCCA
AGTACGCCCCCTATTGACGTCAATGACGGTAAATGGCCCGCCTGGCATTGTGCCCAGTACATGACCTTATGGGACTTTCC
TACTTGGCAGTACATCTACGTATTAGTCATCGCTATTACCATGGTCGAGGTGAGCCCCACGTTCTGCTTCACTCTCCCCA
TCTCCCCCCCCTCCCCACCCCCAATTTTGTATTTATTTATTTTTTAATTATTTTGTGCAGCGATGGGGGCGGGGGGGGGG
GGGGGGGCGCGCGCCAGGCGGGGCGGGGCGGGGGCGAGGGGGGGGCGGGGCGAGGCGGAGAGGTGCGGCGGCAGCCAATC
AGAGCGGCGCGCTCCGAAAGTTTCCTTTTATGGCGAGGCGGCGGCGGCGGCGGCCCTATAAAAAGCGAAGCGCGCGGCGG
GCGGGAGTCGCTGCGCGCTGCCTTCGCCCCGTGCCCCGCTCCGCCGCCGCCTCGCGCCGCCCGCCCCGGCTCTGACTGAC
CGCGTTACTCCCACAGGTGAGCGGGCGGGACGGCCCTTCTCCTCCGGGCTGTAATTAGCTGAGCAAGAGGTAAGGGTTTA
AGGGATGGTTGGTTGGTGGGGTATTAATGTTTAATTACCTGGAGCACCTGCCTGAAATCACTTTTTTTCAGGTTGGACCG
GTCGAATTCCCCGGGGATCCTCTAGAGTCGACGCCACCATGCCGTACCTTGGCTCCGAGGACGTGGTGAAGGAGCTGAAG
AAGGCTCTGTGCAATCCTCACATTCAAGCTGATAGGCTGCGCTACCGGAATGTCATCCAGCGAGTGATTAGGTACATGAC
TCAAGGCTTGGACATGTCTGGTGTTTTTATGGAAATGGTGAAGGCCAGTGCCACTGTAGATATTGTCCAGAAGAAGTTGG
TTTATCTGTACATGTGCACATATGCTCCCCTGAAACCAGATCTGGCTCTCCTGGCCATCAATACGCTGTGCAAAGACTGC
TCAGACCCCAATCCAATGGTGCGAGGGCTGGCGTTACGGAGCATGTGTAGCCTCAGGATGCCTGGTGTGCAGGAGTATAT
ACAACAGCCTATTCTCAATGGTCTGCGGGATAAGGCTTCATATGTCAGGAGAGTGGCAGTCCTTGGATGTGCCAAGATGC
ATAATCTTCATGGAGACTCTGAAGTAGATGGTGCCCTGGTAAATGAATTATACAGTTTGCTGCGTGACCAGGATCCAATT
GTAGTTGTGAACTGCTTGAGGTCTCTAGAGGAAATTCTGAAACAGGAAGGAGGCGTTGTCATCAATAAGCCCATTGCTCA
CCATCTCTTAAATCGAATGTCAAAACTGGACCAATGGGGCCAGGCTGAAGTATTGAACTTTCTGCTACGCTACCAACCCC
GCAGTGAGGAAGAACTATTTGACATTCTCAATCTGTTGGATAGTTTCCTCAAGAGCAGTAGCCCAGGTGTGGTGATGGGA
GCTACCAAACTTTTTCTGATCTTGGCAAAAATGTTTCCCCACGTACAAACTGATGTCCTTGTGCGGGTCAAGGGACCTTT
GCTAGCTGCCTGTTCTTCAGAGAGCCGTGAGCTCTGTTTTGTTGCTCTTTGTCATGTACGCCAGATCTTGCATAGTTTAC
CAGGTCACTTTAGCAGCCACTACAAAAAGTTTTTTTGCTCCTACTCGGAGCCCCACTACATCAAACTACAGAAAGTGGAG
GTGCTGTGTGAACTGGTGAACGATGAGAATGTGCAGCAGGTGCTAGAGGAGCTTCGAGGGTACTGCACGGATGTGTCTGC
GGACTTTGCACAGGCTGCCATCTTTGCCATAGGTGGCATTGCCAGGACTTACACAGATCAATGTGTTCAGATTTTAACAG
AGTTGCTGGGTCTTCGACAAGAGCACATTACCACAGTGGTGGTGCAGACTTTCCGAGACCTGGTTTGGTTGTGTCCTCAG
TGTACTGAAGCTGTATGTCAGGCCCTGCCCGGCTGTGAAGAGAACATTCAAGATAGTGAGGGGAAGCAAGCACTTATTTG
GCTACTTGGTGTCCATGGGGAAAGAATTCCTAATGCTCCTTATGTGTTAGAGGACTTTGTTGAGAATGTGAAGTCGGAAA
CATTTCCAGCTGTTAAGATGGAGCTGCTCACTGCTTTGCTGCGCCTTTTCCTCTCCCGACCTGCTGAGTGCCAGGACATG
CTAGGACGTTTGTTGTATTACTGCATAGAGGAAGAAAAAGATATGGCTGTACGGGACCGAGGTCTCTTCTATTATCGCCT
CCTCTTAGTTGGCATTGATGAAGTTAAGCGGATTCTGTGTAGCCCTAAATCTGACCCTACTCTTGGACTTTTGGAGGATC
CGGCAGAAAGACCTGTGAATAGCTGGGCCTCAGACTTCAACACACTGGTGCCAGTGTATGGCAAAGCCCACTGGGCAACT
ATCTCTAAATGCCAGGGGGCAGAGCGTTGTGACCCAGAGCTTCCTAAAACTTCATCCTTTGCCGCATCAGGACCCTTGAT
TCCTGAAGAGAACAAGGAGAGGGTACAAGAACTCCCTGATTCTGGAGCCCTCATGCTAGTCCCCAATCGCCAGCTTACTG
CTGATTATTTTGAGAAAACTTGGCTTAGCCTTAAAGTTGCTCATCAGCAAGTGTTGCCTTGGCGGGGAGAATTCCATCCT
GACACCCTCCAGATGGCTCTTCAAGTAGTGAACATCCAGACCATCGCAATGAGTAGGGCTGGGTCTCGGCCATGGAAAGC
ATACCTCAGTGCTCAGGATGATACTGGCTGTCTGTTCTTAACAGAACTGCTATTGGAGCCTGGAAACTCAGAAATGCAGA
TCTCTGTGAAACAAAATGAAGCAAGAACGGAGACGCTGAATAGTTTTATTTCTGTATTAGAAACTGTGATTGGAACAATT
GAAGAAATAAAATCATAAGCGGCCGCAAGCTTGCCTCGAGAGATCTACGGGTGGCATCCCTGTGACCCCTCCCCAGTGCC
TCTCCTGGCCCTGGAAGTTGCCACTCCAGTGCCCACCAGCCTTGTCCTAATAAAATTAAGTTGCATCATTTTGTCTGACT
AGGTGTCCTTCTATAATATTATGGGGTGGAGGGGGGTGGTATGGAGCAAGGGGCAAGTTGGGAAGACAACCTGTAGGGCC
TGCGGGGTCTATTGGGAACCAAGCTGGAGTGCAGTGGCACAATCTTGGCTCACTGCAATCTCCGCCTCCTGGGTTCAAGC
GATTCTCCTGCCTCAGCCTCCCGAGTTGTTGGGATTCCAGGCATGCATGACCAGGCTCAGCTAATTTTTGTTTTTTTGGT
AGAGACGGGGTTTCACCATATTGGCCAGGCTGGTCTCCAACTCCTAATCTCAGGTGATCTACCCACCTTGGCCTCCCAAA
TTGCTGGGATTACAGGCGTGAACCACTGCTCCCTTCCCTGTCCTTCTGATTTTGTAGGTAACCACGTGCGGACCGAGCGG
CCGCAGGAACCCCTAGTGATGGAGTTGGCCACTCCCTCTCTGCGCGCTCGCTCGCTCACTGAGGCCGGGCGACCAAAGGT
CGCCCGACGCCCGGGCTTTGCCCGGGCGGCCTCAGTGAGCGAGCGAGCGCGCAGCTGCCTGCAGGGGCGCCTGATGCGGT
ATTTTCTCCTTACGCATCTGTGCGGTATTTCACACCGCATACGTCAAAGCAACCATAGTACGCGCCCTGTAGCGGCGCAT
TAAGCGCGGCGGGTGTGGTGGTTACGCGCAGCGTGACCGCTACACTTGCCAGCGCCTTAGCGCCCGCTCCTTTCGCTTTC
TTCCCTTCCTTTCTCGCCACGTTCGCCGGCTTTCCCCGTCAAGCTCTAAATCGGGGGCTCCCTTTAGGGTTCCGATTTAG
TGCTTTACGGCACCTCGACCCCAAAAAACTTGATTTGGGTGATGGTTCACGTAGTGGGCCATCGCCCTGATAGACGGTTT
TTCGCCCTTTGACGTTGGAGTCCACGTTCTTTAATAGTGGACTCTTGTTCCAAACTGGAACAACACTCAACTCTATCTCG
GGCTATTCTTTTGATTTATAAGGGATTTTGCCGATTTCGGTCTATTGGTTAAAAAATGAGCTGATTTAACAAAAATTTAA
CGCGAATTTTAACAAAATATTAACGTTTACAATTTTATGGTGCACTCTCAGTACAATCTGCTCTGATGCCGCATAGTTAA
GCCAGCCCCGACACCCGCCAACACCCGCTGACGCGCCCTGACGGGCTTGTCTGCTCCCGGCATCCGCTTACAGACAAGCT
GTGACCGTCTCCGGGAGCTGCATGTGTCAGAGGTTTTCACCGTCATCACCGAAACGCGCGAGACGAAAGGGCCTCGTGAT
ACGCCTATTTTTATAGGTTAATGTCATGACACGTAGAAAGCCAGTCCGCAGAAACGGTGCTGACCCCGGATGAATGTCAG
CTACTGGGCTATCTGGACAAGGGAAAACGCAAGCGCAAAGAGAAAGCAGGTAGCTTGCAGTGGGCTTACATGGCGATAGC
TAGACTGGGCGGTTTTATGGACAGCAAGCGAACCGGAATTGCCAGCTGGGGCGCCCTCTGGTAAGGTTGGGAAGCCCTGC
AAAGTAAACTGGATGGCTTTCTTGCCGCCAAGGATCTGATGGCGCAGGGGATCAAGATCTGATCAAGAGACAGGATGAGG
ATCGTTTCGCATGATTGAACAAGATGGATTGCACGCAGGTTCTCCGGCCGCTTGGGTGGAGAGGCTATTCGGCTATGACT
GGGCACAACAGACAATCGGCTGCTCTGATGCCGCCGTGTTCCGGCTGTCAGCGCAGGGGCGCCCGGTTCTTTTTGTCAAG
ACCGACCTGTCCGGTGCCCTGAATGAACTGCAGGACGAGGCAGCGCGGCTATCGTGGCTGGCCACGACGGGCGTTCCTTG
CGCAGCTGTGCTCGACGTTGTCACTGAAGCGGGAAGGGACTGGCTGCTATTGGGCGAAGTGCCGGGGCAGGATCTCCTGT
CATCCCACCTTGCTCCTGCCGAGAAAGTATCCATCATGGCTGATGCAATGCGGCGGCTGCATACGCTTGATCCGGCTACC
TGCCCATTCGACCACCAAGCGAAACATCGCATCGAGCGAGCACGTACTCGGATGGAAGCCGGTCTTGTCGATCAGGATGA
TCTGGACGAAGAGCATCAGGGGCTCGCGCCAGCCGAACTGTTCGCCAGGCTCAAGGCGCGCATGCCCGACGGCGAGGATC
TCGTCGTGACCCATGGCGATGCCTGCTTGCCGAATATCATGGTGGAAAATGGCCGCTTTTCTGGATTCATCGACTGTGGC
CGGCTGGGTGTGGCGGACCGCTATCAGGACATAGCGTTGGCTACCCGTGATATTGCTGAAGAGCTTGGCGGCGAATGGGC
TGACCGCTTCCTCGTGCTTTACGGTATCGCCGCTCCCGATTCGCAGCGCATCGCCTTCTATCGCCTTCTTGACGAGTTCT
TCTGAATTTCATGACCAAAATCCCTTAACGTGAGTTTTCGTTCCACTGAGCGTCAGACCCCGTAGAAAAGATCAAAGGAT
CTTCTTGAGATCCTTTTTTTCTGCGCGTAATCTGCTGCTTGCAAACAAAAAAACCACCGCTACCAGCGGTGGTTTGTTTG
CCGGATCAAGAGCTACCAACTCTTTTTCCGAAGGTAACTGGCTTCAGCAGAGCGCAGATACCAAATACTGTTCTTCTAGT
GTAGCCGTAGTTAGGCCACCACTTCAAGAACTCTGTAGCACCGCCTACATACCTCGCTCTGCTAATCCTGTTACCAGTGG
CTGCTGCCAGTGGCGATAAGTCGTGTCTTACCGGGTTGGACTCAAGACGATAGTTACCGGATAAGGCGCAGCGGTCGGGC
TGAACGGGGGGTTCGTGCACACAGCCCAGCTTGGAGCGAACGACCTACACCGAACTGAGATACCTACAGCGTGAGCTATG
AGAAAGCGCCACGCTTCCCGAAGGGAGAAAGGCGGACAGGTATCCGGTAAGCGGCAGGGTCGGAACAGGAGAGCGCACGA
GGGAGCTTCCAGGGGGAAACGCCTGGTATCTTTATAGTCCTGTCGGGTTTCGCCACCTCTGACTTGAGCGTCGATTTTTG
TGATGCTCGTCAGGGGGGCGGAGCCTATGGAAAAACGCCAGCAACGCGGCCTTTTTACGGTTCCTGGCCTTTTGCTGGCC
TTTTGCTCACATGT
SEQ ID NO 24 AAV-Syn1-hAP4B1-hGH polyA-Kan
CCTGCAGGCAGCTGCGCGCTCGCTCGCTCACTGAGGCCGCCCGGGCAAAGCCCGGGCGTCGGGCGACCTTTGGTCGCCCG
GCCTCAGTGAGCGAGCGAGCGCGCAGAGAGGGAGTGGCCAACTCCATCACTAGGGGTTCCTGCGGCCGCACGCGTCCagt
gcaagtgggttttaggaccaggatgaggcggggtgggggtgcctacctgacgaccgaccccgacccactggacaagcacc
caacccccattccccaaattgcgcatcccctatcagagagggggaggggaaacaggatgcggcgaggcgcgtgcgcactg
ccagcttcagcaccgcggacagtgccttcgcccccgcctggcggcgcgcgccaccgccgcctcagcactgaaggcgcgct
gacgtcactcgccggtcccccgcaaactccccttcccggccaccttggtcgcgtccgcgccgccgccggcccagccggac
cgcaccacgcgaggcgcgagataggggggcacgggcgcgaccatctgcgctgcggcgccggcgactcagcgctgcctcag
tctgcggtgggcagcggaggagtcgtgtcgtgcctgagagcgcagGTTGGACCGGTCGAATTCCCCGGGGATCCTCTAGA
GTCGACGCCACCATGCCGTACCTTGGCTCCGAGGACGTGGTGAAGGAGCTGAAGAAGGCTCTGTGCAATCCTCACATTCA
AGCTGATAGGCTGCGCTACCGGAATGTCATCCAGCGAGTGATTAGGTACATGACTCAAGGCTTGGACATGTCTGGTGTTT
TTATGGAAATGGTGAAGGCCAGTGCCACTGTAGATATTGTCCAGAAGAAGTTGGTTTATCTGTACATGTGCACATATGCT
CCCCTGAAACCAGATCTGGCTCTCCTGGCCATCAATACGCTGTGCAAAGACTGCTCAGACCCCAATCCAATGGTGCGAGG
GCTGGCGTTACGGAGCATGTGTAGCCTCAGGATGCCTGGTGTGCAGGAGTATATACAACAGCCTATTCTCAATGGTCTGC
GGGATAAGGCTTCATATGTCAGGAGAGTGGCAGTCCTTGGATGTGCCAAGATGCATAATCTTCATGGAGACTCTGAAGTA
GATGGTGCCCTGGTAAATGAATTATACAGTTTGCTGCGTGACCAGGATCCAATTGTAGTTGTGAACTGCTTGAGGTCTCT
AGAGGAAATTCTGAAACAGGAAGGAGGCGTTGTCATCAATAAGCCCATTGCTCACCATCTCTTAAATCGAATGTCAAAAC
TGGACCAATGGGGCCAGGCTGAAGTATTGAACTTTCTGCTACGCTACCAACCCCGCAGTGAGGAAGAACTATTTGACATT
CTCAATCTGTTGGATAGTTTCCTCAAGAGCAGTAGCCCAGGTGTGGTGATGGGAGCTACCAAACTTTTTCTGATCTTGGC
AAAAATGTTTCCCCACGTACAAACTGATGTCCTTGTGCGGGTCAAGGGACCTTTGCTAGCTGCCTGTTCTTCAGAGAGCC
GTGAGCTCTGTTTTGTTGCTCTTTGTCATGTACGCCAGATCTTGCATAGTTTACCAGGTCACTTTAGCAGCCACTACAAA
AAGTTTTTTTGCTCCTACTCGGAGCCCCACTACATCAAACTACAGAAAGTGGAGGTGCTGTGTGAACTGGTGAACGATGA
GAATGTGCAGCAGGTGCTAGAGGAGCTTCGAGGGTACTGCACGGATGTGTCTGCGGACTTTGCACAGGCTGCCATCTTTG
CCATAGGTGGCATTGCCAGGACTTACACAGATCAATGTGTTCAGATTTTAACAGAGTTGCTGGGTCTTCGACAAGAGCAC
ATTACCACAGTGGTGGTGCAGACTTTCCGAGACCTGGTTTGGTTGTGTCCTCAGTGTACTGAAGCTGTATGTCAGGCCCT
GCCCGGCTGTGAAGAGAACATTCAAGATAGTGAGGGGAAGCAAGCACTTATTTGGCTACTTGGTGTCCATGGGGAAAGAA
TTCCTAATGCTCCTTATGTGTTAGAGGACTTTGTTGAGAATGTGAAGTCGGAAACATTTCCAGCTGTTAAGATGGAGCTG
CTCACTGCTTTGCTGCGCCTTTTCCTCTCCCGACCTGCTGAGTGCCAGGACATGCTAGGACGTTTGTTGTATTACTGCAT
AGAGGAAGAAAAAGATATGGCTGTACGGGACCGAGGTCTCTTCTATTATCGCCTCCTCTTAGTTGGCATTGATGAAGTTA
AGCGGATTCTGTGTAGCCCTAAATCTGACCCTACTCTTGGACTTTTGGAGGATCCGGCAGAAAGACCTGTGAATAGCTGG
GCCTCAGACTTCAACACACTGGTGCCAGTGTATGGCAAAGCCCACTGGGCAACTATCTCTAAATGCCAGGGGGCAGAGCG
TTGTGACCCAGAGCTTCCTAAAACTTCATCCTTTGCCGCATCAGGACCCTTGATTCCTGAAGAGAACAAGGAGAGGGTAC
AAGAACTCCCTGATTCTGGAGCCCTCATGCTAGTCCCCAATCGCCAGCTTACTGCTGATTATTTTGAGAAAACTTGGCTT
AGCCTTAAAGTTGCTCATCAGCAAGTGTTGCCTTGGCGGGGAGAATTCCATCCTGACACCCTCCAGATGGCTCTTCAAGT
AGTGAACATCCAGACCATCGCAATGAGTAGGGCTGGGTCTCGGCCATGGAAAGCATACCTCAGTGCTCAGGATGATACTG
GCTGTCTGTTCTTAACAGAACTGCTATTGGAGCCTGGAAACTCAGAAATGCAGATCTCTGTGAAACAAAATGAAGCAAGA
ACGGAGACGCTGAATAGTTTTATTTCTGTATTAGAAACTGTGATTGGAACAATTGAAGAAATAAAATCATAAGCGGCCGC
AAGCTTGCCTCGAGAGATCTACGGGTGGCATCCCTGTGACCCCTCCCCAGTGCCTCTCCTGGCCCTGGAAGTTGCCACTC
CAGTGCCCACCAGCCTTGTCCTAATAAAATTAAGTTGCATCATTTTGTCTGACTAGGTGTCCTTCTATAATATTATGGGG
TGGAGGGGGGTGGTATGGAGCAAGGGGCAAGTTGGGAAGACAACCTGTAGGGCCTGCGGGGTCTATTGGGAACCAAGCTG
GAGTGCAGTGGCACAATCTTGGCTCACTGCAATCTCCGCCTCCTGGGTTCAAGCGATTCTCCTGCCTCAGCCTCCCGAGT
TGTTGGGATTCCAGGCATGCATGACCAGGCTCAGCTAATTTTTGTTTTTTTGGTAGAGACGGGGTTTCACCATATTGGCC
AGGCTGGTCTCCAACTCCTAATCTCAGGTGATCTACCCACCTTGGCCTCCCAAATTGCTGGGATTACAGGCGTGAACCAC
TGCTCCCTTCCCTGTCCTTCTGATTTTGTAGGTAACCACGTGCGGACCGAGCGGCCGCAGGAACCCCTAGTGATGGAGTT
GGCCACTCCCTCTCTGCGCGCTCGCTCGCTCACTGAGGCCGGGCGACCAAAGGTCGCCCGACGCCCGGGCTTTGCCCGGG
CGGCCTCAGTGAGCGAGCGAGCGCGCAGCTGCCTGCAGGGGCGCCTGATGCGGTATTTTCTCCTTACGCATCTGTGCGGT
ATTTCACACCGCATACGTCAAAGCAACCATAGTACGCGCCCTGTAGCGGCGCATTAAGCGCGGCGGGTGTGGTGGTTACG
CGCAGCGTGACCGCTACACTTGCCAGCGCCTTAGCGCCCGCTCCTTTCGCTTTCTTCCCTTCCTTTCTCGCCACGTTCGC
CGGCTTTCCCCGTCAAGCTCTAAATCGGGGGCTCCCTTTAGGGTTCCGATTTAGTGCTTTACGGCACCTCGACCCCAAAA
AACTTGATTTGGGTGATGGTTCACGTAGTGGGCCATCGCCCTGATAGACGGTTTTTCGCCCTTTGACGTTGGAGTCCACG
TTCTTTAATAGTGGACTCTTGTTCCAAACTGGAACAACACTCAACTCTATCTCGGGCTATTCTTTTGATTTATAAGGGAT
TTTGCCGATTTCGGTCTATTGGTTAAAAAATGAGCTGATTTAACAAAAATTTAACGCGAATTTTAACAAAATATTAACGT
TTACAATTTTATGGTGCACTCTCAGTACAATCTGCTCTGATGCCGCATAGTTAAGCCAGCCCCGACACCCGCCAACACCC
GCTGACGCGCCCTGACGGGCTTGTCTGCTCCCGGCATCCGCTTACAGACAAGCTGTGACCGTCTCCGGGAGCTGCATGTG
TCAGAGGTTTTCACCGTCATCACCGAAACGCGCGAGACGAAAGGGCCTCGTGATACGCCTATTTTTATAGGTTAATGTCA
TGACACGTAGAAAGCCAGTCCGCAGAAACGGTGCTGACCCCGGATGAATGTCAGCTACTGGGCTATCTGGACAAGGGAAA
ACGCAAGCGCAAAGAGAAAGCAGGTAGCTTGCAGTGGGCTTACATGGCGATAGCTAGACTGGGCGGTTTTATGGACAGCA
AGCGAACCGGAATTGCCAGCTGGGGCGCCCTCTGGTAAGGTTGGGAAGCCCTGCAAAGTAAACTGGATGGCTTTCTTGCC
GCCAAGGATCTGATGGCGCAGGGGATCAAGATCTGATCAAGAGACAGGATGAGGATCGTTTCGCATGATTGAACAAGATG
GATTGCACGCAGGTTCTCCGGCCGCTTGGGTGGAGAGGCTATTCGGCTATGACTGGGCACAACAGACAATCGGCTGCTCT
GATGCCGCCGTGTTCCGGCTGTCAGCGCAGGGGCGCCCGGTTCTTTTTGTCAAGACCGACCTGTCCGGTGCCCTGAATGA
ACTGCAGGACGAGGCAGCGCGGCTATCGTGGCTGGCCACGACGGGCGTTCCTTGCGCAGCTGTGCTCGACGTTGTCACTG
AAGCGGGAAGGGACTGGCTGCTATTGGGCGAAGTGCCGGGGCAGGATCTCCTGTCATCCCACCTTGCTCCTGCCGAGAAA
GTATCCATCATGGCTGATGCAATGCGGCGGCTGCATACGCTTGATCCGGCTACCTGCCCATTCGACCACCAAGCGAAACA
TCGCATCGAGCGAGCACGTACTCGGATGGAAGCCGGTCTTGTCGATCAGGATGATCTGGACGAAGAGCATCAGGGGCTCG
CGCCAGCCGAACTGTTCGCCAGGCTCAAGGCGCGCATGCCCGACGGCGAGGATCTCGTCGTGACCCATGGCGATGCCTGC
TTGCCGAATATCATGGTGGAAAATGGCCGCTTTTCTGGATTCATCGACTGTGGCCGGCTGGGTGTGGCGGACCGCTATCA
GGACATAGCGTTGGCTACCCGTGATATTGCTGAAGAGCTTGGCGGCGAATGGGCTGACCGCTTCCTCGTGCTTTACGGTA
TCGCCGCTCCCGATTCGCAGCGCATCGCCTTCTATCGCCTTCTTGACGAGTTCTTCTGAATTTCATGACCAAAATCCCTT
AACGTGAGTTTTCGTTCCACTGAGCGTCAGACCCCGTAGAAAAGATCAAAGGATCTTCTTGAGATCCTTTTTTTCTGCGC
GTAATCTGCTGCTTGCAAACAAAAAAACCACCGCTACCAGCGGTGGTTTGTTTGCCGGATCAAGAGCTACCAACTCTTTT
TCCGAAGGTAACTGGCTTCAGCAGAGCGCAGATACCAAATACTGTTCTTCTAGTGTAGCCGTAGTTAGGCCACCACTTCA
AGAACTCTGTAGCACCGCCTACATACCTCGCTCTGCTAATCCTGTTACCAGTGGCTGCTGCCAGTGGCGATAAGTCGTGT
CTTACCGGGTTGGACTCAAGACGATAGTTACCGGATAAGGCGCAGCGGTCGGGCTGAACGGGGGGTTCGTGCACACAGCC
CAGCTTGGAGCGAACGACCTACACCGAACTGAGATACCTACAGCGTGAGCTATGAGAAAGCGCCACGCTTCCCGAAGGGA
GAAAGGCGGACAGGTATCCGGTAAGCGGCAGGGTCGGAACAGGAGAGCGCACGAGGGAGCTTCCAGGGGGAAACGCCTGG
TATCTTTATAGTCCTGTCGGGTTTCGCCACCTCTGACTTGAGCGTCGATTTTTGTGATGCTCGTCAGGGGGGCGGAGCCT
ATGGAAAAACGCCAGCAACGCGGCCTTTTTACGGTTCCTGGCCTTTTGCTGGCCTTTTGCTCACATGT
SEQ ID NO 25 AAV-endog.AP4B1-hAP4B1-hGH polyA-Kan
CCTGCAGGCAGCTGCGCGCTCGCTCGCTCACTGAGGCCGCCCGGGCAAAGCCCGGGCGTCGGGCGACCTTTGGTCGCCCG
GCCTCAGTGAGCGAGCGAGCGCGCAGAGAGGGAGTGGCCAACTCCATCACTAGGGGTTCCTGCGGCCGCACGCGTCCggc
ttccacaacggcgtcccgataaaagtcatgctggggcaagaacagggccagagaccctaagggaaagagcccagcagagc
aataagttaccagagtgggcagaaaaaggaagtcaccctgcccgctggagcccaaatcgcggagggcgctggacactccc
aaccgcgcgggagcggaggccgagccgggcactcaaagcgcgcgctgcagggaggagacaggccaaagtagccgatttcc
gattgcccgccgcagctccaccagagggagccgcgcagctgcgcctggagagtgagaggttgggggaagggcatcctaag
gtcggagtcgggactacgcttagcgctcgctcgcggggagaccggcagcggtcttctgtttggtctcttgacaagggggc
gggacttccgcagaaagcaagattgggcctatccgcgatgagaacggcgtggagggggaattaaggctggaactggagag
ggaggggtctgcggtcagtcggacgcggcgctggaaatccggcggaggacctagaagtagcgggagcgatctgtgggcgg
ggcgaacggcctgccggccgacggtagggcctgaagaGTTGGACCGGTCGAATTCCCCGGGGATCCTCTAGAGTCGACGC
CACCATGCCGTACCTTGGCTCCGAGGACGTGGTGAAGGAGCTGAAGAAGGCTCTGTGCAATCCTCACATTCAAGCTGATA
GGCTGCGCTACCGGAATGTCATCCAGCGAGTGATTAGGTACATGACTCAAGGCTTGGACATGTCTGGTGTTTTTATGGAA
ATGGTGAAGGCCAGTGCCACTGTAGATATTGTCCAGAAGAAGTTGGTTTATCTGTACATGTGCACATATGCTCCCCTGAA
ACCAGATCTGGCTCTCCTGGCCATCAATACGCTGTGCAAAGACTGCTCAGACCCCAATCCAATGGTGCGAGGGCTGGCGT
TACGGAGCATGTGTAGCCTCAGGATGCCTGGTGTGCAGGAGTATATACAACAGCCTATTCTCAATGGTCTGCGGGATAAG
GCTTCATATGTCAGGAGAGTGGCAGTCCTTGGATGTGCCAAGATGCATAATCTTCATGGAGACTCTGAAGTAGATGGTGC
CCTGGTAAATGAATTATACAGTTTGCTGCGTGACCAGGATCCAATTGTAGTTGTGAACTGCTTGAGGTCTCTAGAGGAAA
TTCTGAAACAGGAAGGAGGCGTTGTCATCAATAAGCCCATTGCTCACCATCTCTTAAATCGAATGTCAAAACTGGACCAA
TGGGGCCAGGCTGAAGTATTGAACTTTCTGCTACGCTACCAACCCCGCAGTGAGGAAGAACTATTTGACATTCTCAATCT
GTTGGATAGTTTCCTCAAGAGCAGTAGCCCAGGTGTGGTGATGGGAGCTACCAAACTTTTTCTGATCTTGGCAAAAATGT
TTCCCCACGTACAAACTGATGTCCTTGTGCGGGTCAAGGGACCTTTGCTAGCTGCCTGTTCTTCAGAGAGCCGTGAGCTC
TGTTTTGTTGCTCTTTGTCATGTACGCCAGATCTTGCATAGTTTACCAGGTCACTTTAGCAGCCACTACAAAAAGTTTTT
TTGCTCCTACTCGGAGCCCCACTACATCAAACTACAGAAAGTGGAGGTGCTGTGTGAACTGGTGAACGATGAGAATGTGC
AGCAGGTGCTAGAGGAGCTTCGAGGGTACTGCACGGATGTGTCTGCGGACTTTGCACAGGCTGCCATCTTTGCCATAGGT
GGCATTGCCAGGACTTACACAGATCAATGTGTTCAGATTTTAACAGAGTTGCTGGGTCTTCGACAAGAGCACATTACCAC
AGTGGTGGTGCAGACTTTCCGAGACCTGGTTTGGTTGTGTCCTCAGTGTACTGAAGCTGTATGTCAGGCCCTGCCCGGCT
GTGAAGAGAACATTCAAGATAGTGAGGGGAAGCAAGCACTTATTTGGCTACTTGGTGTCCATGGGGAAAGAATTCCTAAT
GCTCCTTATGTGTTAGAGGACTTTGTTGAGAATGTGAAGTCGGAAACATTTCCAGCTGTTAAGATGGAGCTGCTCACTGC
TTTGCTGCGCCTTTTCCTCTCCCGACCTGCTGAGTGCCAGGACATGCTAGGACGTTTGTTGTATTACTGCATAGAGGAAG
AAAAAGATATGGCTGTACGGGACCGAGGTCTCTTCTATTATCGCCTCCTCTTAGTTGGCATTGATGAAGTTAAGCGGATT
CTGTGTAGCCCTAAATCTGACCCTACTCTTGGACTTTTGGAGGATCCGGCAGAAAGACCTGTGAATAGCTGGGCCTCAGA
CTTCAACACACTGGTGCCAGTGTATGGCAAAGCCCACTGGGCAACTATCTCTAAATGCCAGGGGGCAGAGCGTTGTGACC
CAGAGCTTCCTAAAACTTCATCCTTTGCCGCATCAGGACCCTTGATTCCTGAAGAGAACAAGGAGAGGGTACAAGAACTC
CCTGATTCTGGAGCCCTCATGCTAGTCCCCAATCGCCAGCTTACTGCTGATTATTTTGAGAAAACTTGGCTTAGCCTTAA
AGTTGCTCATCAGCAAGTGTTGCCTTGGCGGGGAGAATTCCATCCTGACACCCTCCAGATGGCTCTTCAAGTAGTGAACA
TCCAGACCATCGCAATGAGTAGGGCTGGGTCTCGGCCATGGAAAGCATACCTCAGTGCTCAGGATGATACTGGCTGTCTG
TTCTTAACAGAACTGCTATTGGAGCCTGGAAACTCAGAAATGCAGATCTCTGTGAAACAAAATGAAGCAAGAACGGAGAC
GCTGAATAGTTTTATTTCTGTATTAGAAACTGTGATTGGAACAATTGAAGAAATAAAATCATAAGCGGCCGCAAGCTTGC
CTCGAGAGATCTACGGGTGGCATCCCTGTGACCCCTCCCCAGTGCCTCTCCTGGCCCTGGAAGTTGCCACTCCAGTGCCC
ACCAGCCTTGTCCTAATAAAATTAAGTTGCATCATTTTGTCTGACTAGGTGTCCTTCTATAATATTATGGGGTGGAGGGG
GGTGGTATGGAGCAAGGGGCAAGTTGGGAAGACAACCTGTAGGGCCTGCGGGGTCTATTGGGAACCAAGCTGGAGTGCAG
TGGCACAATCTTGGCTCACTGCAATCTCCGCCTCCTGGGTTCAAGCGATTCTCCTGCCTCAGCCTCCCGAGTTGTTGGGA
TTCCAGGCATGCATGACCAGGCTCAGCTAATTTTTGTTTTTTTGGTAGAGACGGGGTTTCACCATATTGGCCAGGCTGGT
CTCCAACTCCTAATCTCAGGTGATCTACCCACCTTGGCCTCCCAAATTGCTGGGATTACAGGCGTGAACCACTGCTCCCT
TCCCTGTCCTTCTGATTTTGTAGGTAACCACGTGCGGACCGAGCGGCCGCAGGAACCCCTAGTGATGGAGTTGGCCACTC
CCTCTCTGCGCGCTCGCTCGCTCACTGAGGCCGGGCGACCAAAGGTCGCCCGACGCCCGGGCTTTGCCCGGGCGGCCTCA
GTGAGCGAGCGAGCGCGCAGCTGCCTGCAGGGGCGCCTGATGCGGTATTTTCTCCTTACGCATCTGTGCGGTATTTCACA
CCGCATACGTCAAAGCAACCATAGTACGCGCCCTGTAGCGGCGCATTAAGCGCGGCGGGTGTGGTGGTTACGCGCAGCGT
GACCGCTACACTTGCCAGCGCCTTAGCGCCCGCTCCTTTCGCTTTCTTCCCTTCCTTTCTCGCCACGTTCGCCGGCTTTC
CCCGTCAAGCTCTAAATCGGGGGCTCCCTTTAGGGTTCCGATTTAGTGCTTTACGGCACCTCGACCCCAAAAAACTTGAT
TTGGGTGATGGTTCACGTAGTGGGCCATCGCCCTGATAGACGGTTTTTCGCCCTTTGACGTTGGAGTCCACGTTCTTTAA
TAGTGGACTCTTGTTCCAAACTGGAACAACACTCAACTCTATCTCGGGCTATTCTTTTGATTTATAAGGGATTTTGCCGA
TTTCGGTCTATTGGTTAAAAAATGAGCTGATTTAACAAAAATTTAACGCGAATTTTAACAAAATATTAACGTTTACAATT
TTATGGTGCACTCTCAGTACAATCTGCTCTGATGCCGCATAGTTAAGCCAGCCCCGACACCCGCCAACACCCGCTGACGC
GCCCTGACGGGCTTGTCTGCTCCCGGCATCCGCTTACAGACAAGCTGTGACCGTCTCCGGGAGCTGCATGTGTCAGAGGT
TTTCACCGTCATCACCGAAACGCGCGAGACGAAAGGGCCTCGTGATACGCCTATTTTTATAGGTTAATGTCATGACACGT
AGAAAGCCAGTCCGCAGAAACGGTGCTGACCCCGGATGAATGTCAGCTACTGGGCTATCTGGACAAGGGAAAACGCAAGC
GCAAAGAGAAAGCAGGTAGCTTGCAGTGGGCTTACATGGCGATAGCTAGACTGGGCGGTTTTATGGACAGCAAGCGAACC
GGAATTGCCAGCTGGGGCGCCCTCTGGTAAGGTTGGGAAGCCCTGCAAAGTAAACTGGATGGCTTTCTTGCCGCCAAGGA
TCTGATGGCGCAGGGGATCAAGATCTGATCAAGAGACAGGATGAGGATCGTTTCGCATGATTGAACAAGATGGATTGCAC
GCAGGTTCTCCGGCCGCTTGGGTGGAGAGGCTATTCGGCTATGACTGGGCACAACAGACAATCGGCTGCTCTGATGCCGC
CGTGTTCCGGCTGTCAGCGCAGGGGCGCCCGGTTCTTTTTGTCAAGACCGACCTGTCCGGTGCCCTGAATGAACTGCAGG
ACGAGGCAGCGCGGCTATCGTGGCTGGCCACGACGGGCGTTCCTTGCGCAGCTGTGCTCGACGTTGTCACTGAAGCGGGA
AGGGACTGGCTGCTATTGGGCGAAGTGCCGGGGCAGGATCTCCTGTCATCCCACCTTGCTCCTGCCGAGAAAGTATCCAT
CATGGCTGATGCAATGCGGCGGCTGCATACGCTTGATCCGGCTACCTGCCCATTCGACCACCAAGCGAAACATCGCATCG
AGCGAGCACGTACTCGGATGGAAGCCGGTCTTGTCGATCAGGATGATCTGGACGAAGAGCATCAGGGGCTCGCGCCAGCC
GAACTGTTCGCCAGGCTCAAGGCGCGCATGCCCGACGGCGAGGATCTCGTCGTGACCCATGGCGATGCCTGCTTGCCGAA
TATCATGGTGGAAAATGGCCGCTTTTCTGGATTCATCGACTGTGGCCGGCTGGGTGTGGCGGACCGCTATCAGGACATAG
CGTTGGCTACCCGTGATATTGCTGAAGAGCTTGGCGGCGAATGGGCTGACCGCTTCCTCGTGCTTTACGGTATCGCCGCT
CCCGATTCGCAGCGCATCGCCTTCTATCGCCTTCTTGACGAGTTCTTCTGAATTTCATGACCAAAATCCCTTAACGTGAG
TTTTCGTTCCACTGAGCGTCAGACCCCGTAGAAAAGATCAAAGGATCTTCTTGAGATCCTTTTTTTCTGCGCGTAATCTG
CTGCTTGCAAACAAAAAAACCACCGCTACCAGCGGTGGTTTGTTTGCCGGATCAAGAGCTACCAACTCTTTTTCCGAAGG
TAACTGGCTTCAGCAGAGCGCAGATACCAAATACTGTTCTTCTAGTGTAGCCGTAGTTAGGCCACCACTTCAAGAACTCT
GTAGCACCGCCTACATACCTCGCTCTGCTAATCCTGTTACCAGTGGCTGCTGCCAGTGGCGATAAGTCGTGTCTTACCGG
GTTGGACTCAAGACGATAGTTACCGGATAAGGCGCAGCGGTCGGGCTGAACGGGGGGTTCGTGCACACAGCCCAGCTTGG
AGCGAACGACCTACACCGAACTGAGATACCTACAGCGTGAGCTATGAGAAAGCGCCACGCTTCCCGAAGGGAGAAAGGCG
GACAGGTATCCGGTAAGCGGCAGGGTCGGAACAGGAGAGCGCACGAGGGAGCTTCCAGGGGGAAACGCCTGGTATCTTTA
TAGTCCTGTCGGGTTTCGCCACCTCTGACTTGAGCGTCGATTTTTGTGATGCTCGTCAGGGGGGCGGAGCCTATGGAAAA
ACGCCAGCAACGCGGCCTTTTTACGGTTCCTGGCCTTTTGCTGGCCTTTTGCTCACATGT
SEQ ID NO 26 AAV-JeT-hAP4B1-hGH polyA-Kan
CCTGCAGGCAGCTGCGCGCTCGCTCGCTCACTGAGGCCGCCCGGGCAAAGCCCGGGCGTCGGGCGACCTTTGGTCGCCCG
GCCTCAGTGAGCGAGCGAGCGCGCAGAGAGGGAGTGGCCAACTCCATCACTAGGGGTTCCTGCGGCCGCACGCGTCCggg
cggagttagggcggagccaatcagcgtgcgccgttccgaaagttgccttttatggctgggcggagaatgggcggtgaacg
ccgatgattatataaggacgcgccgggtgtggcacagctagttccgtcgcagccgggatttgggtcgcggttcttgtttg
tGTTGGACCGGTCGAATTCCCCGGGGATCCTCTAGAGTCGACGCCACCATGCCGTACCTTGGCTCCGAGGACGTGGTGAA
GGAGCTGAAGAAGGCTCTGTGCAATCCTCACATTCAAGCTGATAGGCTGCGCTACCGGAATGTCATCCAGCGAGTGATTA
GGTACATGACTCAAGGCTTGGACATGTCTGGTGTTTTTATGGAAATGGTGAAGGCCAGTGCCACTGTAGATATTGTCCAG
AAGAAGTTGGTTTATCTGTACATGTGCACATATGCTCCCCTGAAACCAGATCTGGCTCTCCTGGCCATCAATACGCTGTG
CAAAGACTGCTCAGACCCCAATCCAATGGTGCGAGGGCTGGCGTTACGGAGCATGTGTAGCCTCAGGATGCCTGGTGTGC
AGGAGTATATACAACAGCCTATTCTCAATGGTCTGCGGGATAAGGCTTCATATGTCAGGAGAGTGGCAGTCCTTGGATGT
GCCAAGATGCATAATCTTCATGGAGACTCTGAAGTAGATGGTGCCCTGGTAAATGAATTATACAGTTTGCTGCGTGACCA
GGATCCAATTGTAGTTGTGAACTGCTTGAGGTCTCTAGAGGAAATTCTGAAACAGGAAGGAGGCGTTGTCATCAATAAGC
CCATTGCTCACCATCTCTTAAATCGAATGTCAAAACTGGACCAATGGGGCCAGGCTGAAGTATTGAACTTTCTGCTACGC
TACCAACCCCGCAGTGAGGAAGAACTATTTGACATTCTCAATCTGTTGGATAGTTTCCTCAAGAGCAGTAGCCCAGGTGT
GGTGATGGGAGCTACCAAACTTTTTCTGATCTTGGCAAAAATGTTTCCCCACGTACAAACTGATGTCCTTGTGCGGGTCA
AGGGACCTTTGCTAGCTGCCTGTTCTTCAGAGAGCCGTGAGCTCTGTTTTGTTGCTCTTTGTCATGTACGCCAGATCTTG
CATAGTTTACCAGGTCACTTTAGCAGCCACTACAAAAAGTTTTTTTGCTCCTACTCGGAGCCCCACTACATCAAACTACA
GAAAGTGGAGGTGCTGTGTGAACTGGTGAACGATGAGAATGTGCAGCAGGTGCTAGAGGAGCTTCGAGGGTACTGCACGG
ATGTGTCTGCGGACTTTGCACAGGCTGCCATCTTTGCCATAGGTGGCATTGCCAGGACTTACACAGATCAATGTGTTCAG
ATTTTAACAGAGTTGCTGGGTCTTCGACAAGAGCACATTACCACAGTGGTGGTGCAGACTTTCCGAGACCTGGTTTGGTT
GTGTCCTCAGTGTACTGAAGCTGTATGTCAGGCCCTGCCCGGCTGTGAAGAGAACATTCAAGATAGTGAGGGGAAGCAAG
CACTTATTTGGCTACTTGGTGTCCATGGGGAAAGAATTCCTAATGCTCCTTATGTGTTAGAGGACTTTGTTGAGAATGTG
AAGTCGGAAACATTTCCAGCTGTTAAGATGGAGCTGCTCACTGCTTTGCTGCGCCTTTTCCTCTCCCGACCTGCTGAGTG
CCAGGACATGCTAGGACGTTTGTTGTATTACTGCATAGAGGAAGAAAAAGATATGGCTGTACGGGACCGAGGTCTCTTCT
ATTATCGCCTCCTCTTAGTTGGCATTGATGAAGTTAAGCGGATTCTGTGTAGCCCTAAATCTGACCCTACTCTTGGACTT
TTGGAGGATCCGGCAGAAAGACCTGTGAATAGCTGGGCCTCAGACTTCAACACACTGGTGCCAGTGTATGGCAAAGCCCA
CTGGGCAACTATCTCTAAATGCCAGGGGGCAGAGCGTTGTGACCCAGAGCTTCCTAAAACTTCATCCTTTGCCGCATCAG
GACCCTTGATTCCTGAAGAGAACAAGGAGAGGGTACAAGAACTCCCTGATTCTGGAGCCCTCATGCTAGTCCCCAATCGC
CAGCTTACTGCTGATTATTTTGAGAAAACTTGGCTTAGCCTTAAAGTTGCTCATCAGCAAGTGTTGCCTTGGCGGGGAGA
ATTCCATCCTGACACCCTCCAGATGGCTCTTCAAGTAGTGAACATCCAGACCATCGCAATGAGTAGGGCTGGGTCTCGGC
CATGGAAAGCATACCTCAGTGCTCAGGATGATACTGGCTGTCTGTTCTTAACAGAACTGCTATTGGAGCCTGGAAACTCA
GAAATGCAGATCTCTGTGAAACAAAATGAAGCAAGAACGGAGACGCTGAATAGTTTTATTTCTGTATTAGAAACTGTGAT
TGGAACAATTGAAGAAATAAAATCATAAGCGGCCGCAAGCTTGCCTCGAGAGATCTACGGGTGGCATCCCTGTGACCCCT
CCCCAGTGCCTCTCCTGGCCCTGGAAGTTGCCACTCCAGTGCCCACCAGCCTTGTCCTAATAAAATTAAGTTGCATCATT
TTGTCTGACTAGGTGTCCTTCTATAATATTATGGGGTGGAGGGGGGTGGTATGGAGCAAGGGGCAAGTTGGGAAGACAAC
CTGTAGGGCCTGCGGGGTCTATTGGGAACCAAGCTGGAGTGCAGTGGCACAATCTTGGCTCACTGCAATCTCCGCCTCCT
GGGTTCAAGCGATTCTCCTGCCTCAGCCTCCCGAGTTGTTGGGATTCCAGGCATGCATGACCAGGCTCAGCTAATTTTTG
TTTTTTTGGTAGAGACGGGGTTTCACCATATTGGCCAGGCTGGTCTCCAACTCCTAATCTCAGGTGATCTACCCACCTTG
GCCTCCCAAATTGCTGGGATTACAGGCGTGAACCACTGCTCCCTTCCCTGTCCTTCTGATTTTGTAGGTAACCACGTGCG
GACCGAGCGGCCGCAGGAACCCCTAGTGATGGAGTTGGCCACTCCCTCTCTGCGCGCTCGCTCGCTCACTGAGGCCGGGC
GACCAAAGGTCGCCCGACGCCCGGGCTTTGCCCGGGCGGCCTCAGTGAGCGAGCGAGCGCGCAGCTGCCTGCAGGGGCGC
CTGATGCGGTATTTTCTCCTTACGCATCTGTGCGGTATTTCACACCGCATACGTCAAAGCAACCATAGTACGCGCCCTGT
AGCGGCGCATTAAGCGCGGCGGGTGTGGTGGTTACGCGCAGCGTGACCGCTACACTTGCCAGCGCCTTAGCGCCCGCTCC
TTTCGCTTTCTTCCCTTCCTTTCTCGCCACGTTCGCCGGCTTTCCCCGTCAAGCTCTAAATCGGGGGCTCCCTTTAGGGT
TCCGATTTAGTGCTTTACGGCACCTCGACCCCAAAAAACTTGATTTGGGTGATGGTTCACGTAGTGGGCCATCGCCCTGA
TAGACGGTTTTTCGCCCTTTGACGTTGGAGTCCACGTTCTTTAATAGTGGACTCTTGTTCCAAACTGGAACAACACTCAA
CTCTATCTCGGGCTATTCTTTTGATTTATAAGGGATTTTGCCGATTTCGGTCTATTGGTTAAAAAATGAGCTGATTTAAC
AAAAATTTAACGCGAATTTTAACAAAATATTAACGTTTACAATTTTATGGTGCACTCTCAGTACAATCTGCTCTGATGCC
GCATAGTTAAGCCAGCCCCGACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACCGAAACGCGCGAGACGAAAGG
GCCTCGTGATACGCCTATTTTTATAGGTTAATGTCATGACACGTAGAAAGCCAGTCCGCAGAAACGGTGCTGACCCCGGA
TGAATGTCAGCTACTGGGCTATCTGGACAAGGGAAAACGCAAGCGCAAAGAGAAAGCAGGTAGCTTGCAGTGGGCTTACA
TGGCGATAGCTAGACTGGGCGGTTTTATGGACAGCAAGCGAACCGGAATTGCCAGCTGGGGCGCCCTCTGGTAAGGTTGG
GAAGCCCTGCAAAGTAAACTGGATGGCTTTCTTGCCGCCAAGGATCTGATGGCGCAGGGGATCAAGATCTGATCAAGAGA
CAGGATGAGGATCGTTTCGCATGATTGAACAAGATGGATTGCACGCAGGTTCTCCGGCCGCTTGGGTGGAGAGGCTATTC
GGCTATGACTGGGCACAACAGACAATCGGCTGCTCTGATGCCGCCGTGTTCCGGCTGTCAGCGCAGGGGCGCCCGGTTCT
TTTTGTCAAGACCGACCTGTCCGGTGCCCTGAATGAACTGCAGGACGAGGCAGCGCGGCTATCGTGGCTGGCCACGACGG
GCGTTCCTTGCGCAGCTGTGCTCGACGTTGTCACTGAAGCGGGAAGGGACTGGCTGCTATTGGGCGAAGTGCCGGGGCAG
GATCTCCTGTCATCCCACCTTGCTCCTGCCGAGAAAGTATCCATCATGGCTGATGCAATGCGGCGGCTGCATACGCTTGA
TCCGGCTACCTGCCCATTCGACCACCAAGCGAAACATCGCATCGAGCGAGCACGTACTCGGATGGAAGCCGGTCTTGTCG
ATCAGGATGATCTGGACGAAGAGCATCAGGGGCTCGCGCCAGCCGAACTGTTCGCCAGGCTCAAGGCGCGCATGCCCGAC
GGCGAGGATCTCGTCGTGACCCATGGCGATGCCTGCTTGCCGAATATCATGGTGGAAAATGGCCGCTTTTCTGGATTCAT
CGACTGTGGCCGGCTGGGTGTGGCGGACCGCTATCAGGACATAGCGTTGGCTACCCGTGATATTGCTGAAGAGCTTGGCG
GCGAATGGGCTGACCGCTTCCTCGTGCTTTACGGTATCGCCGCTCCCGATTCGCAGCGCATCGCCTTCTATCGCCTTCTT
GACGAGTTCTTCTGAATTTCATGACCAAAATCCCTTAACGTGAGTTTTCGTTCCACTGAGCGTCAGACCCCGTAGAAAAG
ATCAAAGGATCTTCTTGAGATCCTTTTTTTCTGCGCGTAATCTGCTGCTTGCAAACAAAAAAACCACCGCTACCAGCGGT
GGTTTGTTTGCCGGATCAAGAGCTACCAACTCTTTTTCCGAAGGTAACTGGCTTCAGCAGAGCGCAGATACCAAATACTG
TTCTTCTAGTGTAGCCGTAGTTAGGCCACCACTTCAAGAACTCTGTAGCACCGCCTACATACCTCGCTCTGCTAATCCTG
TTACCAGTGGCTGCTGCCAGTGGCGATAAGTCGTGTCTTACCGGGTTGGACTCAAGACGATAGTTACCGGATAAGGCGCA
GCGGTCGGGCTGAACGGGGGGTTCGTGCACACAGCCCAGCTTGGAGCGAACGACCTACACCGAACTGAGATACCTACAGC
GTGAGCTATGAGAAAGCGCCACGCTTCCCGAAGGGAGAAAGGCGGACAGGTATCCGGTAAGCGGCAGGGTCGGAACAGGA
GAGCGCACGAGGGAGCTTCCAGGGGGAAACGCCTGGTATCTTTATAGTCCTGTCGGGTTTCGCCACCTCTGACTTGAGCG
TCGATTTTTGTGATGCTCGTCAGGGGGGCGGAGCCTATGGAAAAACGCCAGCAACGCGGCCTTTTTACGGTTCCTGGCCT
TTTGCTGGCCTTTTGCTCACATGT
SEQ ID NO 27 AAV-MeP229-hAP4B1-hGH polyA-Kan
CCTGCAGGCAGCTGCGCGCTCGCTCGCTCACTGAGGCCGCCCGGGCAAAGCCCGGGCGTCGGGCGACCTTTGGTCGCCCG
GCCTCAGTGAGCGAGCGAGCGCGCAGAGAGGGAGTGGCCAACTCCATCACTAGGGGTTCCTGCGGCCGCACGCGTCCcaa
ttgagggcgtcaccgctaaggctccgccccagcctgggctccacaaccaatgaagggtaatctcgacaaagagcaagggg
tggggcgcgggcgcgcaggtgcagcagcacacaggctggtcgggagggcggggcgcgacgtctgccgtgcggggtcccgg
catcggttgcgcgcgcgctccctcctctcggagagagggctgtggtaaaacccgtccggaaaGTTGGACCGGTCGAATTC
CCCGGGGATCCTCTAGAGTCGACGCCACCATGCCGTACCTTGGCTCCGAGGACGTGGTGAAGGAGCTGAAGAAGGCTCTG
TGCAATCCTCACATTCAAGCTGATAGGCTGCGCTACCGGAATGTCATCCAGCGAGTGATTAGGTACATGACTCAAGGCTT
GGACATGTCTGGTGTTTTTATGGAAATGGTGAAGGCCAGTGCCACTGTAGATATTGTCCAGAAGAAGTTGGTTTATCTGT
ACATGTGCACATATGCTCCCCTGAAACCAGATCTGGCTCTCCTGGCCATCAATACGCTGTGCAAAGACTGCTCAGACCCC
AATCCAATGGTGCGAGGGCTGGCGTTACGGAGCATGTGTAGCCTCAGGATGCCTGGTGTGCAGGAGTATATACAACAGCC
TATTCTCAATGGTCTGCGGGATAAGGCTTCATATGTCAGGAGAGTGGCAGTCCTTGGATGTGCCAAGATGCATAATCTTC
ATGGAGACTCTGAAGTAGATGGTGCCCTGGTAAATGAATTATACAGTTTGCTGCGTGACCAGGATCCAATTGTAGTTGTG
AACTGCTTGAGGTCTCTAGAGGAAATTCTGAAACAGGAAGGAGGCGTTGTCATCAATAAGCCCATTGCTCACCATCTCTT
AAATCGAATGTCAAAACTGGACCAATGGGGCCAGGCTGAAGTATTGAACTTTCTGCTACGCTACCAACCCCGCAGTGAGG
AAGAACTATTTGACATTCTCAATCTGTTGGATAGTTTCCTCAAGAGCAGTAGCCCAGGTGTGGTGATGGGAGCTACCAAA
CTTTTTCTGATCTTGGCAAAAATGTTTCCCCACGTACAAACTGATGTCCTTGTGCGGGTCAAGGGACCTTTGCTAGCTGC
CTGTTCTTCAGAGAGCCGTGAGCTCTGTTTTGTTGCTCTTTGTCATGTACGCCAGATCTTGCATAGTTTACCAGGTCACT
TTAGCAGCCACTACAAAAAGTTTTTTTGCTCCTACTCGGAGCCCCACTACATCAAACTACAGAAAGTGGAGGTGCTGTGT
GAACTGGTGAACGATGAGAATGTGCAGCAGGTGCTAGAGGAGCTTCGAGGGTACTGCACGGATGTGTCTGCGGACTTTGC
ACAGGCTGCCATCTTTGCCATAGGTGGCATTGCCAGGACTTACACAGATCAATGTGTTCAGATTTTAACAGAGTTGCTGG
GTCTTCGACAAGAGCACATTACCACAGTGGTGGTGCAGACTTTCCGAGACCTGGTTTGGTTGTGTCCTCAGTGTACTGAA
GCTGTATGTCAGGCCCTGCCCGGCTGTGAAGAGAACATTCAAGATAGTGAGGGGAAGCAAGCACTTATTTGGCTACTTGG
TGTCCATGGGGAAAGAATTCCTAATGCTCCTTATGTGTTAGAGGACTTTGTTGAGAATGTGAAGTCGGAAACATTTCCAG
CTGTTAAGATGGAGCTGCTCACTGCTTTGCTGCGCCTTTTCCTCTCCCGACCTGCTGAGTGCCAGGACATGCTAGGACGT
TTGTTGTATTACTGCATAGAGGAAGAAAAAGATATGGCTGTACGGGACCGAGGTCTCTTCTATTATCGCCTCCTCTTAGT
TGGCATTGATGAAGTTAAGCGGATTCTGTGTAGCCCTAAATCTGACCCTACTCTTGGACTTTTGGAGGATCCGGCAGAAA
GACCTGTGAATAGCTGGGCCTCAGACTTCAACACACTGGTGCCAGTGTATGGCAAAGCCCACTGGGCAACTATCTCTAAA
TGCCAGGGGGCAGAGCGTTGTGACCCAGAGCTTCCTAAAACTTCATCCTTTGCCGCATCAGGACCCTTGATTCCTGAAGA
GAACAAGGAGAGGGTACAAGAACTCCCTGATTCTGGAGCCCTCATGCTAGTCCCCAATCGCCAGCTTACTGCTGATTATT
TTGAGAAAACTTGGCTTAGCCTTAAAGTTGCTCATCAGCAAGTGTTGCCTTGGCGGGGAGAATTCCATCCTGACACCCTC
CAGATGGCTCTTCAAGTAGTGAACATCCAGACCATCGCAATGAGTAGGGCTGGGTCTCGGCCATGGAAAGCATACCTCAG
TGCTCAGGATGATACTGGCTGTCTGTTCTTAACAGAACTGCTATTGGAGCCTGGAAACTCAGAAATGCAGATCTCTGTGA
AACAAAATGAAGCAAGAACGGAGACGCTGAATAGTTTTATTTCTGTATTAGAAACTGTGATTGGAACAATTGAAGAAATA
AAATCATAAGCGGCCGCAAGCTTGCCTCGAGAGATCTACGGGTGGCATCCCTGTGACCCCTCCCCAGTGCCTCTCCTGGC
CCTGGAAGTTGCCACTCCAGTGCCCACCAGCCTTGTCCTAATAAAATTAAGTTGCATCATTTTGTCTGACTAGGTGTCCT
TCTATAATATTATGGGGTGGAGGGGGGTGGTATGGAGCAAGGGGCAAGTTGGGAAGACAACCTGTAGGGCCTGCGGGGTC
TATTGGGAACCAAGCTGGAGTGCAGTGGCACAATCTTGGCTCACTGCAATCTCCGCCTCCTGGGTTCAAGCGATTCTCCT
GCCTCAGCCTCCCGAGTTGTTGGGATTCCAGGCATGCATGACCAGGCTCAGCTAATTTTTGTTTTTTTGGTAGAGACGGG
GTTTCACCATATTGGCCAGGCTGGTCTCCAACTCCTAATCTCAGGTGATCTACCCACCTTGGCCTCCCAAATTGCTGGGA
TTACAGGCGTGAACCACTGCTCCCTTCCCTGTCCTTCTGATTTTGTAGGTAACCACGTGCGGACCGAGCGGCCGCAGGAA
CCCCTAGTGATGGAGTTGGCCACTCCCTCTCTGCGCGCTCGCTCGCTCACTGAGGCCGGGCGACCAAAGGTCGCCCGACG
CCCGGGCTTTGCCCGGGCGGCCTCAGTGAGCGAGCGAGCGCGCAGCTGCCTGCAGGGGCGCCTGATGCGGTATTTTCTCC
TTACGCATCTGTGCGGTATTTCACACCGCATACGTCAAAGCAACCATAGTACGCGCCCTGTAGCGGCGCATTAAGCGCGG
CGGGTGTGGTGGTTACGCGCAGCGTGACCGCTACACTTGCCAGCGCCTTAGCGCCCGCTCCTTTCGCTTTCTTCCCTTCC
TTTCTCGCCACGTTCGCCGGCTTTCCCCGTCAAGCTCTAAATCGGGGGCTCCCTTTAGGGTTCCGATTTAGTGCTTTACG
GCACCTCGACCCCAAAAAACTTGATTTGGGTGATGGTTCACGTAGTGGGCCATCGCCCTGATAGACGGTTTTTCGCCCTT
TGACGTTGGAGTCCACGTTCTTTAATAGTGGACTCTTGTTCCAAACTGGAACAACACTCAACTCTATCTCGGGCTATTCT
TTTGATTTATAAGGGATTTTGCCGATTTCGGTCTATTGGTTAAAAAATGAGCTGATTTAACAAAAATTTAACGCGAATTT
TAACAAAATATTAACGTTTACAATTTTATGGTGCACTCTCAGTACAATCTGCTCTGATGCCGCATAGTTAAGCCAGCCCC
GACACCCGCCAACACCCGCTGACGCGCCCTGACGGGCTTGTCTGCTCCCGGCATCCGCTTACAGACAAGCTGTGACCGTC
TCCGGGAGCTGCATGTGTCAGAGGTTTTCACCGTCATCACCGAAACGCGCGAGACGAAAGGGCCTCGTGATACGCCTATT
TTTATAGGTTAATGTCATGACACGTAGAAAGCCAGTCCGCAGAAACGGTGCTGACCCCGGATGAATGTCAGCTACTGGGC
TATCTGGACAAGGGAAAACGCAAGCGCAAAGAGAAAGCAGGTAGCTTGCAGTGGGCTTACATGGCGATAGCTAGACTGGG
CGGTTTTATGGACAGCAAGCGAACCGGAATTGCCAGCTGGGGCGCCCTCTGGTAAGGTTGGGAAGCCCTGCAAAGTAAAC
TGGATGGCTTTCTTGCCGCCAAGGATCTGATGGCGCAGGGGATCAAGATCTGATCAAGAGACAGGATGAGGATCGTTTCG
CATGATTGAACAAGATGGATTGCACGCAGGTTCTCCGGCCGCTTGGGTGGAGAGGCTATTCGGCTATGACTGGGCACAAC
AGACAATCGGCTGCTCTGATGCCGCCGTGTTCCGGCTGTCAGCGCAGGGGCGCCCGGTTCTTTTTGTCAAGACCGACCTG
TCCGGTGCCCTGAATGAACTGCAGGACGAGGCAGCGCGGCTATCGTGGCTGGCCACGACGGGCGTTCCTTGCGCAGCTGT
GCTCGACGTTGTCACTGAAGCGGGAAGGGACTGGCTGCTATTGGGCGAAGTGCCGGGGCAGGATCTCCTGTCATCCCACC
TTGCTCCTGCCGAGAAAGTATCCATCATGGCTGATGCAATGCGGCGGCTGCATACGCTTGATCCGGCTACCTGCCCATTC
GACCACCAAGCGAAACATCGCATCGAGCGAGCACGTACTCGGATGGAAGCCGGTCTTGTCGATCAGGATGATCTGGACGA
AGAGCATCAGGGGCTCGCGCCAGCCGAACTGTTCGCCAGGCTCAAGGCGCGCATGCCCGACGGCGAGGATCTCGTCGTGA
CCCATGGCGATGCCTGCTTGCCGAATATCATGGTGGAAAATGGCCGCTTTTCTGGATTCATCGACTGTGGCCGGCTGGGT
GTGGCGGACCGCTATCAGGACATAGCGTTGGCTACCCGTGATATTGCTGAAGAGCTTGGCGGCGAATGGGCTGACCGCTT
CCTCGTGCTTTACGGTATCGCCGCTCCCGATTCGCAGCGCATCGCCTTCTATCGCCTTCTTGACGAGTTCTTCTGAATTT
CATGACCAAAATCCCTTAACGTGAGTTTTCGTTCCACTGAGCGTCAGACCCCGTAGAAAAGATCAAAGGATCTTCTTGAG
ATCCTTTTTTTCTGCGCGTAATCTGCTGCTTGCAAACAAAAAAACCACCGCTACCAGCGGTGGTTTGTTTGCCGGATCAA
GAGCTACCAACTCTTTTTCCGAAGGTAACTGGCTTCAGCAGAGCGCAGATACCAAATACTGTTCTTCTAGTGTAGCCGTA
GTTAGGCCACCACTTCAAGAACTCTGTAGCACCGCCTACATACCTCGCTCTGCTAATCCTGTTACCAGTGGCTGCTGCCA
GTGGCGATAAGTCGTGTCTTACCGGGTTGGACTCAAGACGATAGTTACCGGATAAGGCGCAGCGGTCGGGCTGAACGGGG
GGTTCGTGCACACAGCCCAGCTTGGAGCGAACGACCTACACCGAACTGAGATACCTACAGCGTGAGCTATGAGAAAGCGC
CACGCTTCCCGAAGGGAGAAAGGCGGACAGGTATCCGGTAAGCGGCAGGGTCGGAACAGGAGAGCGCACGAGGGAGCTTC
CAGGGGGAAACGCCTGGTATCTTTATAGTCCTGTCGGGTTTCGCCACCTCTGACTTGAGCGTCGATTTTTGTGATGCTCG
TCAGGGGGGGGAGCCTATGGAAAAACGCCAGCAACGCGGCCTTTTTACGGTTCCTGGCCTTTTGCTGGCCTTTTGCTCA
CATGT
SEQ ID NO 28 AAV-ChB
CGTTACATAACTTACGGTAAATGGCCCGCCTGGCTGACCGCCCAACGACCCCCGCCCATTGACGTCAATAGTAACGCCAA
TAGGGACTTTCCATTGACGTCAATGGGTGGAGTATTTACGGTAAACTGCCCACTTGGCAGTACATCAAGTGTATCATATG
CCAAGTACGCCCCCTATTGACGTCAATGACGGTAAATGGCCCGCCTGGCATTGTGCCCAGTACATGACCTTATGGGACTT
TCCTACTTGGCAGTACATCTACGTATTAGTCATCGCTATTACCATGGTCGAGGTGAGCCCCACGTTCTGCTTCACTCTCC
CCATCTCCCCCCCCTCCCCACCCCCAATTTTGTATTTATTTATTTTTTAATTATTTTGTGCAGCGATGGGGGGGGGGGGG
GGGGGGGGGGGGCGCGCCAGGCGGGGGGGGGGGGGCGAGGGGCGGGGCGGGGCGAGGCGGAGAGGTGCGGCGGCAGCCA
ATCAGAGCGGCGCGCTCCGAAAGTTTCCTTTTATGGCGAGGCGGCGGCGGCGGCGGCCCTATAAAAAGCGAAGCGCGCGG
CGGGCGGGAGTCGCTGCGCGCTGCCTTCGCCCCGTGCCCCGCTCCGCCGCCGCCTCGCGCCGCCCGCCCCGGCTCTGACT
GACCGCGTTACTCCCACAGGTGAGCGGGCGGGACGGCCCTTCTCCTCCGGGCTGTAATTAGCTGAGCAAGAGGTAAGGGT
TTAAGGGATGGTTGGTTGGTGGGGTATTAATGTTTAATTACCTGGAGCACCTGCCTGAAATCACTTTTTTTCAG

### MATERIALS AND METHODS

### Ethics Statement

All animal in vivo experiments were approved by the University of Sheffield Ethical Review Sub-Committee, the UK Animal Procedures Committee (London, UK) and performed according to the Animal (Scientific Procedures) Act 1986, under the Project License 40/3739. C57BL/6J-Ap4b1^{em5Lutzy}/J mice and non-transgenic C57BL/6J mice were maintained in a controlled facility in a 12h dark/12h light photocycle (on at 7 am/off at 7 pm) with free access to food and water. The ARRIVE guidelines have been followed in reporting this study.

### Viral Vector Construction

The lead clinical vector pAAV-CBh-hAP4B1-Kan (SEQ ID NO 11) was synthesised by Genewiz. Briefly, a CBh promoter (SEQ ID NO 9), the gene of interest (hAP4B1 SEQ ID 15) and a human growth hormone (hGH) poly(A) signal (SEQ ID NO 4), were cloned into a Genewiz plasmid backbone between two AAV2 inverted terminal repeats (ITRs). The CBh promoter was initially designed and described by Grey S J, et al. 2011 as a novel, enhanced, hybrid form of the chicken beta actin (CBA) promoter. CBh is able to provide high level, ubiquitous neuronal expression, including in motor neurons (Gray et al 2011). The CBh promoter is comprised of three parts: a CMV enhancer (SEQ ID NO 1), a chicken beta actin promoter (SEQ ID 3), and a hybrid intron (SEQ ID NO 2) formed from the CBA intron 1 and the minute virus of mice (MVM) VP intron. The CMV enhancer detailed in SEQ ID NO 1 contains an 18 bp deletion compared to the standard CMV enhancer. There are short linker sequences between the 5′ ITR and the CBh promoter, the CBh promoter and hAP4B1, hAP4B1 and the hGH poly(A) signal, and the hGH poly(A) signal and the 3′ ITR. The plasmid backbone also contains an f1 bacteriophage origin of replication downstream of the 3′ITR, a kanamycin resistance gene downstream of this, and a high copy number (pUC) origin of replication immediately upstream of the 5′ ITR. The complete plasmid comprises of 6443 bp with the region to be packaged into AAV9 comprising 3895 bp.

### Initial Safety Study-Cisterna Magna Delivery of Viral Gene Therapy Constructs in P1 Mice.

Post-natal day 1 (P1) wildtype C57BI/6J mice were anaesthetised by isoflurane. Induction occurred in a chamber at 5% isoflurane, 3 L O2/minute. Anaesthesia was maintained via a mask at 1-2% isoflurane, 0.3L O2/minute for approximately 5 minutes during injection. The cisterna magna was located using a Wee-Sight transilluminator vein finder (Phillips). Viral vectors were injected directly into the cisterna magna of P1 mice (n=15 per group), using stereotaxic apparatus containing a 33-gauge Hamilton syringe with automated perfusion pump. The solution was administered at a flow-rate of 1 μL per minute; the maximum volume of solution administered was 5 μL per animal. Animals each received a maximum dose of 5×10¹⁰ total vector genomes. The experimental timeline proceeded as follows:

Day 1-Postnatal day 0, day of birth (PO)-Footpad tattoos applied for identification purposes Day 2-Postnatal day 1 (P1)-Injection of up to 5 L of viral vector or vehicle solution into the cisterna magna, under isoflurane anaesthesia.

Day 29 (or Day 170)-Postnatal day 28 (P28) or P168 (6 months post-injection)-Animals were perfused under terminal anaesthesia, and tissue samples collected for analysis.

### Cisterna Magna Delivery of Viral Gene Therapy Constructs in P1 Mice as Proof-of-Concept.

A study to assess the ability of our therapeutic viral vector to mediate transgene expression in the central nervous system (CNS) of transgenic mice lacking endogenous Ap4b1 (KO C57BL/6J-Ap4b1^{em5Lutzy}/J) after injection via the cisterna magna, was followed. Mice were injected via the cisterna magna as in the previously described safety study. Two viral vectors were used; an AAV9 expressing a full length copy of the human AP4B1 (SPG47) gene and, an AAV9 expressing a V5 tag with no additional coding sequence as a viral control. Mice receiving AAV9-hAP4B1 viral vector were injected with two different doses (a low dose of 2×10¹⁰ vector genomes and a high dose of 4×10¹⁰ vector genomes, respectively), whereas mice receiving AAV9-V5 were injected with a high dose (4×10¹⁰ vector genomes) only. Two more groups were included in the study; untreated KO C57BL/6J-Ap4b1^{em5Lutzy}/J and untreated WT C57BL/6J-Ap4b1^{em5Lutzy}/J. Rescue of the phenotype was assessed by improvements in behavioural parameters, that will be described in details below, in the treated mice compared with untreated.

### Genotyping and Colony Maintenance

C57BL/6J-Ap4b1^{em5Lutzy}/J mice were generated by Jackson Labs using CRISPR-Cas9 mediated deletion of a 76 bp region within Exon 1 of the murine Ap4b1 gene. Deletion of this region generated a frameshift mutation and a truncated mRNA transcript. WT Sequence (deletions in lower case): TTGGCGACGATGCCATAccttggctctgaggacgtggtgaaggaactgaagaaggctctgtgtaaccctcatattcag gctgataggctgcgcTACCGGAATGTCATCCAGCGAGTTATTAGGTATCACCAACCTACCATAG AA.

Genotyping of mice was performed based on the protocol optimised by Charles River Laboratories. Mouse genotyping was performed on genomic DNA extracted from tail or ear tissue by the addition of 20 μl QuickExtract™ DNA Extraction Solution (Lucigen) and incubation on a thermocycler for 15 minutes at 65° C. followed by 2 minutes at 98° C. Genotyping PCRs were performed in a 20 μl volume reaction as separate reactions for WT and KO alleles. Reactions consisted of 5 μl 5×FIREPol® Master Mix Ready to Load with 7.5 mM MgCl₂ (Solis Biodyne), 500 nM each of genotyping primers-P1+P2 for WT allele amplification and P1+P3 for KO allele amplification-(P1: 5′-TCGCCCGAGGACCCAAGAA-3′ (SEQ ID NO 29); P2: 5′-CCTATCAGCCTGAATATGAGGGTTACA-3′ (SEQ ID NO 30); P3: 5′-GCTGGATGACATTCCGGTATATG-3′ (SEQ ID NO 31)) and 1 μl genomic DNA from the QuickExtract™ protocol. Touchdown PCR was performed according to the thermal profile shown in Table 1. Following PCR and agarose gel electrophoresis (2% agarose gel in Tris-acetate-EDTA buffer), WT and KO allele PCR products were visualised at ˜254 bp and ˜203 bp respectively.

Heterozygous mice were bred together to produce homozygous WT (Ap4b1+/+), KO (Ap4b1−/−) and heterozygous (Ap4b1+/−) littermates.

TABLE 1
Touchdown PCR conditions for C57BL/6J-Ap4b1^{em5Lutzy}/J genotyping
PCR conditions

| step | temp [° C.] | time [sec] | |
|---|---|---|---|
| 1) Initial denaturation | 94 | 120 | |
| 2) Denaturation | 94 | 60 | Cycle no. (2-4) × 10 |
| 3) Annealing | TD 58-54 | 60 | |
| 4) Elongation | 72 | 60 | |
| 5) Denaturation | 94 | 60 | Cycle no. (5-7) × 25 |
| 6) Annealing | 54 | 60 | |
| 7) Elongation | 72 | 60 | |
| 8) Final elongation | 72 | 300 | |

### RT-qPCR for Human and Murine AP4B1 Expression Analysis.

RT-qPCR was carried out using 2 μl total RNA diluted to a concentration of 10 ng/μl in nuclease free water, 5 μl 2×QuantiFast SYBR Green RT-PCR Master Mix (Qiagen®), hAP4B1 (Forward: 5′-CTGGTGAACGATGAGAATGT-3′ (SEQ ID No 32); Reverse: 5′-GACCCAGCAACTCTGTTAAA-3′ (SEQ ID No 33), mAp4b1 (Forward: 5′-CTGTGCTAGGCTCCCACATC-3′24 (SEQ ID NO 34); Reverse: 5′-TGGCACTGGCCTTTACCATT-3′ (SEQ ID NO 35) and 18S (forward: 5′ GTAACCCGTTGAACCCCAT 3′ (SEQ ID NO 36); reverse: 5′ CCATCCAATCGGTAGTAGCG 3′ (SEQ ID NO 37) primers (all 1 μM concentration), 0.1 μl QuantiFast RT mix and H₂O to a final volume of 10 μl. Following an initial reverse transcription step at 50° C. for 10 min and a 5 min denaturation step at 95° C., cDNA was amplified by 39 cycles of 95° C. for 10 sec followed by a combined annealing/extension step at 60° C. for 10 sec. This was followed by one cycle at 65° C. for 31 sec, before subsequent melt curve analysis. All RT-qPCR was performed on a Bio-Rad C1000 Touch™ Thermal Cycler. Bio-Rad CFX Manager software was used to analyse signal intensity and relative gene expression values were determined using the ΔΔCt method, with 18S rRNA used as a reference gene.

### Open Field

Open field analysis was performed on mice at ages 6, 9 and 12 months. The protocol followed that performed by Herranz-Martin and colleagues 8. Mice were placed in a translucent box with dimensions 60 cm×40 cm×25 cm. The underside of the box was marked with permanent ink outlining a 5×3 grid of squares. Activity was measured as the number of grid lines crossed by each mouse over a 10 minute period. For a crossing to be recorded, all four paws of the animal were required to cross the grid line. The assessment was carried out in minimal lighting conditions and the apparatus was cleaned with 70% ethanol between each animal. One run was recorded for each animal at each timepoint.

### Rotarod

Ugo Basile 7650 accelerating rotarod (set to accelerate from 3-37 rpm over 300 seconds) was used to measure motor function. Rotarod training was performed over 3 consecutive days, with two trials per day. Subsequently, this test was performed at bi-weekly intervals (characterisation study) or monthly (Proof-of-concept study) in the late morning. For each evaluation, the mice were tested twice, with a minimum rest period of 5 minutes between runs.

The best performance, measured as latency to fall in seconds, was used for analysis. The minimum threshold for recording rotarod activity was 3 seconds.

### Gait Analysis

The CatWalk™ gait analysis system version 7.1 was used to assess gait parameters in Ap4b1-KO and WT mice. Mice were tested at 3, 6, 9 and 12 months of age. Mice were placed on the apparatus in complete darkness and their gait patterns recorded. Six unforced runs were recorded for each mouse and three selected for analysis. The runs to be analysed were selected based on the absence of behavioural anomalies-such as sniffing, exploration and rearing—and where mouse locomotion was consistent and without noticeable accelerations, decelerations or deviations from a straight line. Processing of gait data was performed with the Noldus software. Limbs were assigned manually, and gait parameters were calculated automatically. Parameter values were transferred to GraphPad Prism for statistical analysis.

### Antibodies

Primary antibodies used in this study were mouse anti-α-tubulin (1:5000; Sigma), mouse anti-GAPDH (1:10,000; Millipore), rabbit anti-V5 (1:1000; Abcam), rabbit anti-β4 (in-house non-commercial antibody provided by J. Hirst) (1:400), rabbit anti-ATG9A (1:1000; Abcam), sheep anti-TGN46 (Bio-Rad), anti-MAP2.

### Protein Extraction and Western Blotting for Protein Expression Analysis.

Tissue was harvested from mice under terminal anaesthesia and snap frozen in liquid nitrogen. Tissue was homogenised using a dounce homogeniser in ice-cold RIPA buffer (50 mM Tris-HCL PH 7.4; 1% v/v NP-40; 0.5% w/v sodium deoxycholate; 0.1% v/v SDS; 150 mM NaCl; 2 mM EDTA) containing 1×protease inhibitor cocktail (Sigma-Aldrich). Lysate protein concentrations were determined using the BCA assay (Thermo Scientific Pierce™). 40 μg of protein lysate was denatured by heating to 100° C. for 5 minutes in the presence of 4×loading buffer (10 ml buffer contained: 240 mM Tris-HCL pH 6.8; 8% w/v SDS; 40% glycerol; 0.01% bromophenol blue; 10% β-mercaptoethanol). Lysates that were intended to be used for quantification of ATG9A protein levels were heated to 50° C., as boiling leads to aggregation of ATG9A and loss of signal. Lysates were then loaded onto 4-20% gradient mini-PROTEAN® TGX™ precast polyacrylamide gels (Bio-Rad). Gels were run at 180V in running buffer (25 mM Tris, 192 mM glycine, 0.1% SDS, pH 8.3) for approximately 50 minutes or until the dye front reached the bottom of the gel. Separated proteins were transferred by electrophoresis to an Immobilon-P PVDF membrane (Millipore) that had been pre-soaked in methanol. Protein transfer was carried out at 250 mA for 1.5 hours or 40 mA overnight in transfer buffer (25 mM Tris, 192 mM glycine, 5% v/v methanol). Membranes were blocked for 1 hour in 5% milk/TBS-T. Primary antibodies were diluted in 5% milk/TBS-T or 5% BSA/TBS-T and incubated with the membrane overnight at 4° C. After primary antibody incubation, the membrane was washed for 3×15 min each in TBS-T buffer. Secondary antibodies anti-mouse HRP (1:3000) and anti-rabbit HRP (1:3000) were diluted in 5% milk/TBS-T and incubated with the membrane for 2 hours at room temperature. After secondary antibody incubation, the membrane was washed for 3×15 min each in TBS-T buffer followed by a final 15 min wash in PBS. Protein bands were visualised using ECL Prime Western Blotting Detection Reagent (Amersham) and the G-Box imaging system (Syngene). Densitometric analysis of protein bands was carried out using Image J software.

Western blotting was generated using the following protocol: Cell lysates were extracted as above. 40 μg protein was loaded per lane on 10-well 4-12% Bis-Tris precast gel. The gel was run in 2-(N-morpholino) ethanesulfonic acid (MES) buffer and wet transferred to a nitrocellulose membrane (100 mA constant amps overnight). The membrane was blocked in 5% milk/TBS-T for 1 hour. Primary antibodies were added for 2 hours at room temperature (anti-AP4B1 1:400 in 5% BSA), followed by 4×15-minute washes in PBS-T.

Secondary antibodies were added for 30 mins at room temperature in 5% milk-TBS-T. The membrane was washed 5×5 minutes in PBS-T followed by a 30-minute-long wash in PBS. The membrane was developed with ECL Prime

### Western Blotting Detection Reagent (Amersham).

### Cell Culture

Human Embryonic Kidney (HEK) 293T cells, HeLa-M/HeLa-AP4B1−/−cells (a gift from Dr J. Hirst) and human fibroblast cell lines were cultured at 37° C., 5% CO₂ in growth media consisting of Dulbecco's Modified Eagle's Medium (DMEM, Sigma) supplemented with 10% v/v Fetal Bovine Serum (FBS, Sigma, MI, US) and 1% v/v penicillin (100U/ml) and streptomycin (100U/ml) (Lonza, Basel, Switzerland).

For primary cortical neuron culture, E18 non-transgenic rat embryos and E16 mouse embryos were harvested from wild type and C57BL/6J-Ap4b1^{em5Lutzy}/J pregnant mice, essentially as described by (Krichevsky et al., 2001). Very briefly, the cortices were dissected and digested in 0.25% trypsin in HBSS without calcium or magnesium (GIBCO) at 37° C. for 15 minutes and dissociated manually in triturating medium by using three fire-burnt Pasteur pipettes with successively smaller openings. Dissociated cortical neurons were then plated on poly-D-lysine (SIGMA) coated plates and maintained in Neurobasal medium (Life Technologies) supplemented with 2% B27 (Life Technologies), 0.5 mM GlutaMax (Life Technologies) and 100 U/ml of penicillin and 100 μg/ml streptomycin (Lonza).

AP4B1 knockout Hela cells (HeLa-AP4B1^{-/-}) were provided by Dr J. Hirst, the generation of which is described in ⁹.

The AP4B1 deficient human fibroblasts from SPG47 patients, heterozygous family members, and age-matched homozygous wild type controls were gifted by Dr Henry Houlden and Dr Ivy Pin-Fang Chen.

### Plasmid and Viral Construct Production

AAV2-ITR transgene transfer plasmids-created as described above-were amplified in NEB Stable E. coli cells (New England Biolabs) and purified using Qiagen Plasmid Plus kits. Adenoviral helper genes (pHelper) and Rep-Cap genes (pAAV2/9) were supplied in trans and were obtained commercially through Plasmid Factory. Pseudotyped AAV9 viral vector was produced in-house following the protocol described in ⁶.

### EXAMPLE 1

The size of the human AP4B1 cDNA open reading frame (2,800 bp) means that a simple gene replacement option is technically feasible and amenable to typical viral delivery approaches such as using a single-stranded adeno-associated virus (AAV) which has an insertion limit of ˜4,000 bp. We designed AAV vectors to achieve therapeutic level of transgene expression (FIG. 2A): 1) An expression cassette was developed involving the 0.8 kb CBh promoter and 130 bp SV40 poly A to drive expression of the human AP4B1. The CBh promoter has been reported to mediate efficient transgene expression in rodents and non-human primates; 2) A vector expressing an N-terminal V5 viral epitope-tagged human AP4B1 cDNA allowing in vitro and in vivo detection of AP4B1 restoration in the absence of suitable anti-AP4B1 antibodies; 3) A V5-tagged AP4B1 construct expressed from a lentiviral vector enabling in vitro validation of efficacy in cell types that are not efficiently transduced by AAV9 (e.g. fibroblasts). All constructs have been shown to efficiently express their viral cargos upon transfection or transduction in Hela cells (FIG. 2B,C), primary rat cortical neurons (FIG. 2D), and human fibroblasts (FIG. 2E) as determined by western blotting, RT-qPCR and immunocytochemistry. The viral constructs efficiently restore expression of the AP4B1 protein in both a CRISPR generated AP4B1-knockout HeLa cell line and fibroblasts from SPG47 patients lacking endogenous AP4B1 (FIG. 2 B,C,E). Expression of V5-tagged AP4B1 in SPG47 patient fibroblasts also rescues both overexpression and mis localisation of ATG9A (FIG. 3).

### EXAMPLE 2

To select the appropriate route of delivery for optimal efficacy of the AAV9-CBh-hAP4B1 therapy in the AP4B1^{-/-} mouse model, we designed an in vivo experiment to test the two main delivery paradigms of gene therapies for Central Nervous System (CNS) diseases (FIG. 4): intra-CSF vs intravenous delivery. Indeed, AAV9 has been shown to cross the Blood-Brain Barrier (BBB), particularly when administered in neonates, and it has been delivered intravenously in successful pre-clinical (Valori et al., 2010) and clinical (Mendell et al., 2017) studies of CNS diseases, achieving robust therapeutical potential from this minimally invasive delivery route. However, intra-cerebrospinal fluid (CSF) delivery provides an immediate access to the CNS, therefore increasing the chance of reaching disease-target cells, for which reason it has also been used to deliver gene therapy treatments in pre-clinical (Iannitti et al., 2018) and clinical (Miller et al., 2020; Mueller et al., 2020) studies attempting to treat neurodegenerative diseases.

Based on this, we set out to assess the therapeutic efficacy of AAV9-hAP4B1 either delivered into the CSF, through injection in the cisterna magna (Intra-cisterna magna, ICM), or intravenously through injection in the facial vein (intravenous, IV), in AP4B1^{-/-}P2/P3. As viral control groups, we injected AAV9-V5 only (lacking the hAP4B1 transgene but containing the same CBh promoter, a V5 tag and a poly-A signal) using the same delivery routes, and we also kept wild type (WT) as well as homozygous untreated animals. Considering that our in-house characterisation of the AP4B1^{-/-}mouse model determined that male homozygous have a stronger disease phenotype, we decided to focus our resources by performing this experiment in males only. The following viral vectors used in this experiment were either produced in-house or outsourced to a CRO (VectorBuilder): AAV9-V5 only; AAV9-V5-hAP4B1; and AAV9-untagged hAP4B1. FIG. 4B summarizes the number of pups recruited and sacrificed for this experiment.

Upon perfusion with PBS, tissue was either processed for biochemical or histological analysis.

For biochemical tests, CNS tissue was divided into cerebrum, cerebellum and spinal cord, for a better appreciation of the therapeutic potential of the treatment in distinct and well-defined regions.

We started by tracking the viral biodistribution in the CNS of treated mice by performing qPCR on tissue-extracted genomic DNA with primers binding in the poly-A sequence, with results summarised in FIG. 4C. As determined by qPCR, cisterna magna delivery of AAV9-V5 only and AAV9-V5-hAP4B1 was far superior in transducing all the studied areas of the CNS than intravenous delivery, despite the injections having been done in neonates and AAV9 being able to cross the BBB.

Following biodistribution, we used RT-qPCR on tissue-extracted RNA with primers binding to the hAP4B1 transgene, to assess mRNA expression of the hAP4B1 transgene in the studied tissue, with results summarised in FIG. 5. In line with the results obtained from viral genome biodistribution, cisterna magna injection of AAV9-CBh-hAP4B1 was considerably superior in inducing mRNA expression of hAP4B1 in all CNS regions analysed.

A loss-of-function hypothesis for SPG47 is backed by substantial evidence, as mutations in all the other 3 subunits of the AP4 complex (AP4M1; AP4S1 and AP4E1) disrupt the normal function of the tetrameric protein and cause a very similar clinical presentation to that of SPG47. Therefore, the loss of any of its subunits appears to cause an AP4 deficiency disease, with an early onset progressive spastic paraplegia and intellectual disability as its main characteristics. To investigate whether ICM or IV delivery had restored functionality of the AP-4 complex, we extracted protein from CNS tissue and did Western blot to detect protein expression levels of another AP-4 subunit, AP4E1. Results are summarised in FIG. 7. AP4E1 expression is absent in AP4B1^{-/-}Homozygous untreated mice and animals treated with AAV9-V5 only, but ICM administration of AAV9-V5-hAP4B1 was successful in restoring AP4E1 protein levels in all regions of the CNS analysed, with efficiencies of ˜25% (cerebrum), ˜16% (cerebellum), and ˜36% (spinal cord) of WT levels. Intravenous delivery of AAV9-V5-hAP4B1 did not induce detectable rescue of AP4E1 expression in the cerebrum and cerebellum, whilst inducing a smaller rescue in the spinal cord than ICM injection.

Having collected substantial data from biochemical analysis of the tissue, we proceeded to do some histological analysis, namely, to check the impact of AAV9-hAP4B1 on corpus callosum thickness and enlargement of lateral ventricles in coronal sections of AP4B1-brains, other important hallmark of SPG47 (FIG. 8). Results summarised in FIG. 9 clearly demonstrate that AP4B1 gene replacement led to the rescue of corpus callosum thickness and enlargement of lateral ventricles.

We then proceeded to investigate whether AAV9-hAP4B1 would correct ATG9A mislocalisation, a well-established molecular hallmark of AP-4 neurodegenerative diseases. Immunofluorescence labelling was done on brain sections using anti-ATG9A, results summarised in FIG. 10. As expected, ATG9A shows mislocalisation in homozygous mice when comparing to WT littermates, across all CNS regions analysed. ICM delivery of AAV9-hAP4B1 was successful in correcting this phenotype in the cerebellum and brainstem (FIG. 10). Gene replacement approach was tested using CBh and Synapsin promoters.

Interestingly, our gene therapy approach led to the correction of clasping phenotype observed in Ap4b1−/−mouse model. The data are summarised in FIG. 11.

### EXAMPLE 3

Dose response study investigating the AAV9_CBh_hAP4B1 gene treatment in SPG47 mouse model. SPG47 KO mice (ap4b1−/−) were treated with AAV9_CBh_hAP4B1 or AAV9_CBh_V5_empty vectors around P60 with cisterna magna delivery. 3 different doses of AAV9_CBh_hAP4B1 were delivered—a low-dose (6×10E10 vg), a mid-dose (8×10E10 vg) and a high-dose (1×10E11 vg). Mice are sacrificed at 2 months and 4 months of age. Mice weight and clasping phenotype was assessed weekly, and tissues were taking at 2 months for biochemical analysis and brains were fixed at 4 months for anatomical analysis (corpus callosum and lateral vertical size) and ATG9A accumulation analysis (Table 1).

TABLE 1

| | | | Number | Number |
|---|---|---|---|---|
| | | | of mice 2 | of mice 4 |
| | | Route of | months | months |
| Group | Treatment | delivery | timepoint | timepoint |
| Wildtype | Untreated | | 3 | 9 |
| Ap4b1−/− | AAV9-Empty | Cisterna Magna | 3 | 9 |
| Ap4b1−/− | AAV9-CBh- | Cisterna Magna | 3 | 9 |
| | hAP4B1 | | | |
| | (dose 1 low) | | | |
| Ap4b1−/− | AAV9-CBh- | Cisterna Magna | 3 | 9 |
| | hAP4B1 | | | |
| | (dose 2 mid) | | | |
| Ap4b1−/− | AAV9-CBh- | Cisterna Magna | 3 | 9 |
| | hAP4B1 | | | |
| | (dose 3 high) | | | |

### Weight and Clasping

Treated mice show no adverse effect on weight gain (see FIG. 15). Clasping data displays a progression of hind limb clasping severity over time in SPG47 mice untreated and V5-ony treated (control). Wildtype mice do not show a clasping phenotype with age. Treatment with all 3 doses show a reduction in hind limb clasping severity progression over time (see FIG. 16). Low-dose treatment gives a 86% rescue of hind limb clasping severity at the 120 day timepoint. 76% rescue for mid-dose and a 55% rescue at high-dose treatment.

### Rotarod Latency to Fall

Data on rotarod performance at 6 months of age (4 months post injection). This data displays a possible rescue of this phenotype with both the mid- and high-dose treatments. See FIG. 17.

### Brain Weight

Here we have measured brain weight immediately after dissection of the sacrificed mice. Treated mice (V5-only) have significantly reduce brain weight compared to that of the wildtypes in females at 2 months post treatment. This data suggests a rescue of this brain weight phenotype when treated of all 3 doses (see FIG. 18A); 98% rescue at the low-dose, 85% rescue at the mid-dose and 96% rescue at high-dose treatment. Brain weight measured in males at 4 months post injection show data of a similar pattern. (see FIG. 18B).

### Corpus Callosum Thinning Analysis

Corpus callosum measurements were taken over 8 consistent positions within the brains of treated mice. Corpus callosum thickness varies over these positions. SPG47 control treated mice (V5-only) show a reduced corpus callosum thickness (orange data set) compared to wildtype (blue data set). AAV9_Cbh_hAP4B1 high dose treated mice displays a rescue of the corpus callosum thickness to wild type levels (see FIG. 19).

### EXAMPLE 4

Impact of AAV9-AP4B1 gene replacement on neurofilament L (NFL) levels in cerebrospinal fluid (CSF) and Plasma. As shown in FIG. 19 Ap4b1−/−mice treated with AAV9-CBh-AP4B1 and AAV9-Synapsin 1-AP4B1 vector showed reduction of NFL levels in mice to similar levels seen in wildtype both in CSF and Plasma. The intreated mice shown increased levels when compared to wild type. This indicates that the vector can effectively reduce neurofilament levels in patients lacking AP4B1.

### REFERENCES

1 Harvard SPG47 registry, managed by Dr Darius Ebrahimi-Fakhari, accessed October 2020.
2 Bauer, P. et al. Mutation in the AP4B1 Gene Cause Hereditary Spastic Paraplegia Type47 (SPG47). Neurogenetics 13, 73-76, doi: 10.1007/s10048-012-0314-0. (2012).
3. Laughlin, C. A., Tratschin, J. D., Coon, H. & Carter, B. J. Cloning of infectious adeno-associated virus genomes in bacterial plasmids. Gene 23, 65-73 (1983).
4. Gray, S. J. et al. Optimizing Promoters for Recombinant Adeno-Associated Virus-Mediated Gene Expression in the Peripheral and Central Nervous System Using Self-Complementary Vectors. Hum. Gene Ther. 22, 1143-1153 (2011).
5. Davies, A. K. et al. AP-4 vesicles contribute to spatial control of autophagy via RUSC-dependent peripheral delivery of ATG9A. Nat. Commun. 9, (2018).
6. Lukashchuk, V., Lewis, K. E., Coldicott, I., Grierson, A. J. & Azzouz, M. AAV9-mediated central nervous system-targeted gene delivery via cisterna magna route in mice. Mol. Ther.-Methods Clin. Dev. 3, 15055 (2016).
7. Kim, S., Yu, N. K. & Kaang, B. K. CTCF as a multifunctional protein in genome regulation and gene expression. Exp. Mol. Med. 47, e166 (2015).
8. Herranz-Martin, S. et al. Viral delivery of C9orf72 hexanucleotide repeat expansions in mice leads to repeat-length-dependent neuropathology and behavioural deficits. Dis. Model. Mech. 10, 859-868 (2017).
9. Frazier, M. N. et al. Molecular basis for the interaction between Adaptor Protein Complex 4 (AP4) 34 and its accessory protein, tepsin. Traffic 17, 400-415 (2016).
10. Behne R, Teinert J, Wimmer M, D′Amore A, Davies AK, Scarrott J M, Eberhardt K, Brechmann B, Chen IP, Buttermore ED, Barrett L, Dwyer S, Chen T, Hirst J, Wiesener A, Segal D, Martinuzzi A, Duarte ST, Bennett JT, Bourinaris T, Houlden H, Roubertie A, Santorelli F M, Robinson M, Azzouz M, Lipton J O, Borner G H H, Sahin M, Ebrahimi-Fakhari D. Adaptor protein complex 4 deficiency: a paradigm of childhood-onset hereditary spastic paraplegia caused by defective protein trafficking. Hum Mol Genet. 2020 Jan. 15; 29 (2): 320-334. doi: 10.1093/hmg/ddz310. PMID: 31915823; PMCID: PMC7001721.
11. Ebrahimi-Fakhari D, Teinert J, Behne R, Wimmer M, D′Amore A, Eberhardt K, Brechmann B, Ziegler M, Jensen DM, Nagabhyrava P, Geisel G, Carmody E, Shamshad U, Dies KA, Yuskaitis CJ, Salussolia CL, Ebrahimi-Fakhari D, Pearson TS, Saffari A, Ziegler A, Kölker S, Volkmann J, Wiesener A, Bearden DR, Lakhani S, Segal D, Udwadia-Hegde A, Martinuzzi A, Hirst J, Perlman S, Takiyama Y, Xiromerisiou G, Vill K, Walker WO, Shukla A, Dubey Gupta R, Dahl N, Aksoy A, Verhelst H, Delgado MR, Kremlikova Pourova R, Sadek AA, Elkhateeb NM, Blumkin L, Brea-Fernández AJ, Dacruz-Álvarez D, Smol T, Ghoumid J, Miguel D, Heine C, Schlump JU, Langen H, Baets J, Bulk S, Darvish H, Bakhtiari S, Kruer MC, Lim-Melia E, Aydinli N, Alanay Y, El-Rashidy O, Nampoothiri S, Patel C, Beetz C, Bauer P, Yoon G, Guillot M, Miller SP, Bourinaris T, Houlden H, Robelin L, Anheim M, Alamri AS, Mahmoud AAH, Inaloo S, Habibzadeh P, Faghihi MA, Jansen AC, Brock S, Roubertie A, Darras BT, Agrawal PB, Santorelli FM, Gleeson J, Zaki MS, Sheikh SI, Bennett JT, Sahin M. Defining the clinical, molecular and imaging spectrum of adaptor protein complex 4-associated hereditary spastic paraplegia. Brain. 2020 Oct. 1; 143 (10): 2929-2944. doi: 10.1093/brain/awz307. PMID: 32979048; PMCID: PMC7780481 . . .
12. Orsini, C. A. and B. Setlow, Sex differences in animal models of decision making. J Neurosci Res, 2017. 95 (1-2): p. 260-269.
13. Feng, H., et al., Mouse models of GNAO1-associated movement disorder: Allele-and sex-specific differences in phenotypes. PLOS One, 2019. 14 (1): p. e0211066.
14. McCombe, P. A. and R. D. Henderson, Effects of gender in amyotrophic lateral sclerosis. Gend Med, 2010. 7 (6): p. 557-70.
15. Watkins, J., et al., Female sex mitigates motor and behavioural phenotypes in TDP-43 (Q331K) knock-in mice. Sci Rep, 2020. 10 (1): p. 19220.
16. Sala Frigerio, C., et al., The Major Risk Factors for Alzheimer's Disease: Age, Sex, and Genes Modulate the Microglia Response to AB Plaques. Cell Rep, 2019. 27 (4): p. 1293-1306.e6.
17. Gray SJ, Foti SB, Schwartz J W, Bachaboina L, Taylor-Blake B, Coleman J, Ehlers MD, Zylka M J, Mccown T J, Samulski R J. Optimizing promoters for recombinant adeno-associated virus-mediated gene expression in the peripheral and central nervous system using self-complementary vectors. Hum Gene Ther. 2011 September; 22 (9): 1143-53. doi: 10.1089/hum.2010.245. Epub 2011 Jun. 1. PMID: 21476867

1. An isolated nucleic acid molecule comprising: a transcription cassette comprising in a 5′ to 3′ direction between first and second inverted repeat sequences:
i) a promoter adapted for expression in a mammalian neurone wherein said promoter is associated with an enhancer nucleotide motif;
ii) an intron nucleotide sequence; and
iii) a polyadenylation signal nucleotide sequence; wherein
said cassette further comprises a nucleic acid molecule comprising a nucleotide sequence that encodes at least one protein of the AP-4 complex.

2. The isolated nucleic acid molecule according to claim 1 wherein said enhancer motif is a CMV enhancer.

3. The isolated nucleic acid molecule according to claim 2 wherein said CMV enhancer motif comprises or consists of the nucleotide sequence in SEQ ID NO: 1, or polymorphic nucleotide sequence variant thereof,

4. The isolated nucleic acid molecule according to claim 1, wherein said polyadenylation signal is a growth hormone (GH) polyadenylation signal.

5. The isolated nucleic acid molecule according to claim 4 wherein said GH polyadenylation signal comprises or consists of the nucleotide sequence in SEQ ID NO: 4, or polymorphic sequence variant thereof.

6. The isolated nucleic acid molecule according to claim 1, wherein said promoter is the chicken beta actin promoter, a JeT promoter, a hSyn promoter, a MeP229 promoter, or a AP4B1 promoter.

7. The isolated nucleic acid molecule according to claim 6 wherein said chicken beta actin promoter comprises or consists of the nucleotide sequence in SEQ ID NO:
3 or 28.

8. (canceled)

9. The isolated nucleic acid molecule according to claim 1, wherein said transcription cassette comprises or consists of the nucleotide sequence in SEQ ID NO: 9.

10. The isolated nucleic acid molecule according to claim 1, wherein said transcription cassette comprises a nucleic acid molecule comprising a nucleotide sequence selected from the group consisting of:
i) a nucleotide sequence, or polymorphic sequence variant, as set forth in SEQ ID NO: 15 (AP4B1);
ii) a nucleotide sequence wherein said sequence is degenerate as a result of the genetic code to the nucleotide sequence defined in (i);
iii) a nucleic acid molecule the complementary strand of which hybridizes under stringent hybridization conditions to the sequence in SEQ ID NO: 15 (AP4B1) wherein said nucleic acid molecule encodes a polypeptide that forms a complex with polypeptides comprising the AP-4 complex;
iv) a nucleotide sequence that encodes a polypeptide comprising an amino acid sequence as represented in SEQ ID NO: 16 (AP4B1); and
v) a nucleotide sequence that encodes a polypeptide comprising an amino acid sequence wherein said amino acid sequence is modified by addition deletion or substitution of at least one amino acid residue as represented in iv) wherein said polypeptide forms a complex with polypeptides comprising the AP-4 complex.

11. The isolated nucleic acid molecule according to claim 1, wherein said transcription cassette comprises a nucleic acid molecule comprising a nucleotide sequence selected from the group consisting of:
i) a nucleotide sequence, or polymorphic sequence variant, as set forth in SEQ ID NO: 17 (AP4E1);
ii) a nucleotide sequence wherein said sequence is degenerate as a result of the genetic code to the nucleotide sequence defined in (i);
iii) a nucleic acid molecule the complementary strand of which hybridizes under stringent hybridization conditions to the sequence in SEQ ID NO: 17 (AP4E1); wherein said nucleic acid molecule encodes a polypeptide that forms a complex with polypeptides comprising the AP-4 complex;
iv) a nucleotide sequence that encodes a polypeptide comprising an amino acid sequence as represented in SEQ ID NO: 18 (AP4E1); and
v) a nucleotide sequence that encodes a polypeptide comprising an amino acid sequence wherein said amino acid sequence is modified by addition deletion or substitution of at least one amino acid residue as represented in iv) wherein said polypeptide forms a complex with polypeptides comprising the AP-4 complex.

12. The isolated nucleic acid molecule according to claim 1, wherein claim said transcription cassette comprises a nucleic acid molecule comprising a nucleotide sequence selected from the group consisting of:
i) a nucleotide sequence, or polymorphic sequence variant, as set forth in SEQ ID NO: 19 (AP4M1);
ii) a nucleotide sequence wherein said sequence is degenerate as a result of the genetic code to the nucleotide sequence defined in (i);
iii) a nucleic acid molecule the complementary strand of which hybridizes under stringent hybridization conditions to the sequence in SEQ ID NO: 19(AP4M1); wherein said nucleic acid molecule encodes a polypeptide that forms a complex with polypeptides comprising the AP-4 complex;
iv) a nucleotide sequence that encodes a polypeptide comprising an amino acid sequence as represented in SEQ ID NO: 20 (AP4M1); and
v) a nucleotide sequence that encodes a polypeptide comprising an amino acid sequence wherein said amino acid sequence is modified by addition deletion or substitution of at least one amino acid residue as represented in iv) wherein said polypeptide forms a complex with polypeptides comprising the AP-4 complex.

13. The isolated nucleic acid molecule according to claim 1, wherein said transcription cassette comprises a nucleic acid molecule comprising a nucleotide sequence selected from the group consisting of:
i) a nucleotide sequence, or polymorphic sequence variant, as set forth in SEQ ID NO: 21 (AP4S1);
ii) a nucleotide sequence wherein said sequence is degenerate as a result of the genetic code to the nucleotide sequence defined in (i);
iii) a nucleic acid molecule the complementary strand of which hybridizes under stringent hybridization conditions to the sequence in SEQ ID NO: 21 (AP4S1); wherein said nucleic acid molecule encodes a polypeptide that forms a complex with polypeptides comprising the AP-4 complex; and
iv) a nucleotide sequence that encodes a polypeptide comprising an amino acid sequence as represented in SEQ ID NO: 22 (AP4S1);
a nucleotide sequence that encodes a polypeptide comprising an amino acid sequence wherein said amino acid sequence is modified by addition deletion or substitution of at least one amino acid residue as represented in iv) wherein said polypeptide forms a complex with polypeptides comprising the AP-4 complex.

14. An expression vector comprising the isolated nucleic acid molecule according to claim 1.

15. The expression vector according to claim 14 wherein said expression vector is a viral based expression vector.

16.-20. (canceled)

21. The expression vector according to claim 15 wherein said viral based vector comprises the nucleotide sequence set forth in SEQ ID NO: 10, 11, 12, 13, 14, 23, 24, 25, 26, or 27.

22.-30. (canceled)

31. A pharmaceutical composition comprising the expression vector according to claim 14 and an excipient or carrier.

32. (canceled)

33. A method for treating AP-4 Hereditary Spastic Paraplegias in a subject, comprising administering an effective amount of the expression vector according to claim 14 to the subject.

34. The expression vector according to claim 33 wherein said subject is a paediatric subject.

35. A method for measuring the efficacy of the treatment of hereditary spastic paraplegia in a subject wherein said subject is treated with the expression vector according to claim 14, comprising:
a) measuring the level of neurofilament L (NFL) in a biological sample obtained from the subject suffering from hereditary spastic paraplegia prior to administration of the expression vector, and
b) comparing said levels to the levels of NFL in a biological sample obtained from the subject suffering from hereditary spastic paraplegia after the administration of the expression vector, and wherein i) if the NFL levels are lower when compared to the levels obtained in step a) the treatment with the expression vector or composition is paused, or ii) if the levels are the same as the levels in step a) the treatment with the expression vector is continued.

36. (canceled)

37. The method according to claim 35 wherein said sample under b) is obtained between 1-6 days or 1-4 weeks.

38. A method for measuring efficacy of hereditary spastic paraplegia in a subject suffering from hereditary spastic paraplegia and wherein said subject is treated with the expression vector according to claim 14, comprising:
a) measuring the level of neurofilament L (NFL) in a biological sample obtained from the subject suffering from hereditary spastic paraplegia and treated with said expression vector or composition, and
b) comparing said level with that of control subjects.

39. (canceled)

40. The method according to claim 38 further comprising
c) wherein when said levels in a biological sample obtained from the subject suffering from hereditary spastic paraplegia are the same or lower than the NFL levels in a biological sample obtained from the control subject the treatment is effective and paused, or
c) wherein when said levels in a biological sample obtained from the subject suffering from hereditary spastic paraplegia are higher than the NFL levels in a biological sample obtained from the control subject the treatment is ineffective and the treatment with the expression vector is continued.

41. (canceled)


SEQUENCE LISTING (38 sequences)

SEQ ID NO: 1 - DNA, 287 bp, synthetic construct
  misc_feature 1..287: note = CMV enhancer
  source 1..287: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgg

SEQ ID NO: 2 - DNA, 229 bp, synthetic construct
  misc_feature 1..229: note = Hybrid Intron
  source 1..229: mol_type = other DNA
ggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcag

SEQ ID NO: 3 - DNA, 278 bp, Gallus gallus
  source 1..278: mol_type = genomic DNA
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 4 - DNA, 477 bp, synthetic construct
  misc_feature 1..477: note = GROWTH HORMONE POLYADENYLATION SIGNAL
  source 1..477: mol_type = other DNA
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 5 - DNA, 164 bp, synthetic construct
  misc_feature 1..164: note = JeT promoter
  source 1..164: mol_type = other DNA
gggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgt

SEQ ID NO: 6 - DNA, 448 bp, synthetic construct
  misc_feature 1..448: note = hSyn Promoter
  source 1..448: mol_type = other DNA
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 7 - DNA, 225 bp, synthetic construct
  misc_feature 1..225: note = MeP 229 promoter
  source 1..225: mol_type = other DNA
caattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaa

SEQ ID NO: 8 - DNA, 600 bp, synthetic construct
  misc_feature 1..600: note = AP4B1 promoter
  source 1..600: mol_type = other DNA
ggcttccacaacggcgtcccgataaaagtcatgctggggcaagaacagggccagagaccctaagggaaagagcccagcagagcaataagttaccagagtgggcagaaaaaggaagtcaccctgcccgctggagcccaaatcgcggagggcgctggacactcccaaccgcgcgggagcggaggccgagccgggcactcaaagcgcgcgctgcagggaggagacaggccaaagtagccgatttccgattgcccgccgcagctccaccagagggagccgcgcagctgcgcctggagagtgagaggttgggggaagggcatcctaaggtcggagtcgggactacgcttagcgctcgctcgcggggagaccggcagcggtcttctgtttggtctcttgacaagggggcgggacttccgcagaaagcaagattgggcctatccgcgatgagaacggcgtggagggggaattaaggctggaactggagagggaggggtctgcggtcagtcggacgcggcgctggaaatccggcggaggacctagaagtagcgggagcgatctgtgggcggggcgaacggcctgccggccgacggtagggcctgaaga

SEQ ID NO: 9 - DNA, 794 bp, synthetic construct
  misc_feature 1..794: note = CBh promoter (full)
  source 1..794: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcag

SEQ ID NO: 10 - DNA, 6249 bp, synthetic construct
  misc_feature 1..6249: note = pAAV-AP4B1endog-hAP4B1-Kan
  source 1..6249: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtccggcttccacaacggcgtcccgataaaagtcatgctggggcaagaacagggccagagaccctaagggaaagagcccagcagagcaataagttaccagagtgggcagaaaaaggaagtcaccctgcccgctggagcccaaatcgcggagggcgctggacactcccaaccgcgcgggagcggaggccgagccgggcactcaaagcgcgcgctgcagggaggagacaggccaaagtagccgatttccgattgcccgccgcagctccaccagagggagccgcgcagctgcgcctggagagtgagaggttgggggaagggcatcctaaggtcggagtcgggactacgcttagcgctcgctcgcggggagaccggcagcggtcttctgtttggtctcttgacaagggggcgggacttccgcagaaagcaagattgggcctatccgcgatgagaacggcgtggagggggaattaaggctggaactggagagggaggggtctgcggtcagtcggacgcggcgctggaaatccggcggaggacctagaagtagcgggagcgatctgtgggcggggcgaacggcctgccggccgacggtagggcctgaagaggaattccccggggatcctctagagtcgacgccaccatgccgtaccttggctccgaggacgtggtgaaggagctgaagaaggctctgtgcaatcctcacattcaagctgataggctgcgctaccggaatgtcatccagcgagtgattaggtacatgactcaaggcttggacatgtctggtgtttttatggaaatggtgaaggccagtgccactgtagatattgtccagaagaagttggtttatctgtacatgtgcacatatgctcccctgaaaccagatctggctctcctggccatcaatacgctgtgcaaagactgctcagaccccaatccaatggtgcgagggctggcgttacggagcatgtgtagcctcaggatgcctggtgtgcaggagtatatacaacagcctattctcaatggtctgcgggataaggcttcatatgtcaggagagtggcagtccttggatgtgccaagatgcataatcttcatggagactctgaagtagatggtgccctggtaaatgaattatacagtttgctgcgtgaccaggatccaattgtagttgtgaactgcttgaggtctctagaggaaattctgaaacaggaaggaggcgttgtcatcaataagcccattgctcaccatctcttaaatcgaatgtcaaaactggaccaatggggccaggctgaagtattgaactttctgctacgctaccaaccccgcagtgaggaagaactatttgacattctcaatctgttggatagtttcctcaagagcagtagcccaggtgtggtgatgggagctaccaaactttttctgatcttggcaaaaatgtttccccacgtacaaactgatgtccttgtgcgggtcaagggacctttgctagctgcctgttcttcagagagccgtgagctctgttttgttgctctttgtcatgtacgccagatcttgcatagtttaccaggtcactttagcagccactacaaaaagtttttttgctcctactcggagccccactacatcaaactacagaaagtggaggtgctgtgtgaactggtgaacgatgagaatgtgcagcaggtgctagaggagcttcgagggtactgcacggatgtgtctgcggactttgcacaggctgccatctttgccataggtggcattgccaggacttacacagatcaatgtgttcagattttaacagagttgctgggtcttcgacaagagcacattaccacagtggtggtgcagactttccgagacctggtttggttgtgtcctcagtgtactgaagctgtatgtcaggccctgcccggctgtgaagagaacattcaagatagtgaggggaagcaagcacttatttggctacttggtgtccatggggaaagaattcctaatgctccttatgtgttagaggactttgttgagaatgtgaagtcggaaacatttccagctgttaagatggagctgctcactgctttgctgcgccttttcctctcccgacctgctgagtgccaggacatgctaggacgtttgttgtattactgcatagaggaagaaaaagatatggctgtacgggaccgaggtctcttctattatcgcctcctcttagttggcattgatgaagttaagcggattctgtgtagccctaaatctgaccctactcttggacttttggaggatccggcagaaagacctgtgaatagctgggcctcagacttcaacacactggtgccagtgtatggcaaagcccactgggcaactatctctaaatgccagggggcagagcgttgtgacccagagcttcctaaaacttcatcctttgccgcatcaggacccttgattcctgaagagaacaaggagagggtacaagaactccctgattctggagccctcatgctagtccccaatcgccagcttactgctgattattttgagaaaacttggcttagccttaaagttgctcatcagcaagtgttgccttggcggggagaattccatcctgacaccctccagatggctcttcaagtagtgaacatccagaccatcgcaatgagtagggctgggtctcggccatggaaagcatacctcagtgctcaggatgatactggctgtctgttcttaacagaactgctattggagcctggaaactcagaaatgcagatctctgtgaaacaaaatgaagcaagaacggagacgctgaatagttttatttctgtattagaaactgtgattggaacaattgaagaaataaaatcataagcggccgcaaagcttgcctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 11 - DNA, 6443 bp, synthetic construct
  misc_feature 1..6443: note = pAAV-CBH-hAP4B1-Kan
  source 1..6443: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcagggaattccccggggatcctctagagtcgacgccaccatgccgtaccttggctccgaggacgtggtgaaggagctgaagaaggctctgtgcaatcctcacattcaagctgataggctgcgctaccggaatgtcatccagcgagtgattaggtacatgactcaaggcttggacatgtctggtgtttttatggaaatggtgaaggccagtgccactgtagatattgtccagaagaagttggtttatctgtacatgtgcacatatgctcccctgaaaccagatctggctctcctggccatcaatacgctgtgcaaagactgctcagaccccaatccaatggtgcgagggctggcgttacggagcatgtgtagcctcaggatgcctggtgtgcaggagtatatacaacagcctattctcaatggtctgcgggataaggcttcatatgtcaggagagtggcagtccttggatgtgccaagatgcataatcttcatggagactctgaagtagatggtgccctggtaaatgaattatacagtttgctgcgtgaccaggatccaattgtagttgtgaactgcttgaggtctctagaggaaattctgaaacaggaaggaggcgttgtcatcaataagcccattgctcaccatctcttaaatcgaatgtcaaaactggaccaatggggccaggctgaagtattgaactttctgctacgctaccaaccccgcagtgaggaagaactatttgacattctcaatctgttggatagtttcctcaagagcagtagcccaggtgtggtgatgggagctaccaaactttttctgatcttggcaaaaatgtttccccacgtacaaactgatgtccttgtgcgggtcaagggacctttgctagctgcctgttcttcagagagccgtgagctctgttttgttgctctttgtcatgtacgccagatcttgcatagtttaccaggtcactttagcagccactacaaaaagtttttttgctcctactcggagccccactacatcaaactacagaaagtggaggtgctgtgtgaactggtgaacgatgagaatgtgcagcaggtgctagaggagcttcgagggtactgcacggatgtgtctgcggactttgcacaggctgccatctttgccataggtggcattgccaggacttacacagatcaatgtgttcagattttaacagagttgctgggtcttcgacaagagcacattaccacagtggtggtgcagactttccgagacctggtttggttgtgtcctcagtgtactgaagctgtatgtcaggccctgcccggctgtgaagagaacattcaagatagtgaggggaagcaagcacttatttggctacttggtgtccatggggaaagaattcctaatgctccttatgtgttagaggactttgttgagaatgtgaagtcggaaacatttccagctgttaagatggagctgctcactgctttgctgcgccttttcctctcccgacctgctgagtgccaggacatgctaggacgtttgttgtattactgcatagaggaagaaaaagatatggctgtacgggaccgaggtctcttctattatcgcctcctcttagttggcattgatgaagttaagcggattctgtgtagccctaaatctgaccctactcttggacttttggaggatccggcagaaagacctgtgaatagctgggcctcagacttcaacacactggtgccagtgtatggcaaagcccactgggcaactatctctaaatgccagggggcagagcgttgtgacccagagcttcctaaaacttcatcctttgccgcatcaggacccttgattcctgaagagaacaaggagagggtacaagaactccctgattctggagccctcatgctagtccccaatcgccagcttactgctgattattttgagaaaacttggcttagccttaaagttgctcatcagcaagtgttgccttggcggggagaattccatcctgacaccctccagatggctcttcaagtagtgaacatccagaccatcgcaatgagtagggctgggtctcggccatggaaagcatacctcagtgctcaggatgatactggctgtctgttcttaacagaactgctattggagcctggaaactcagaaatgcagatctctgtgaaacaaaatgaagcaagaacggagacgctgaatagttttatttctgtattagaaactgtgattggaacaattgaagaaataaaatcataagcggccgcaaagcttgcctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 12 - DNA, 6097 bp, synthetic construct
  misc_feature 1..6097: note = pAAV-hSYN1-hAP4B1-Kan
  source 1..6097: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtccagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggaattccccggggatcctctagagtcgacgccaccatgccgtaccttggctccgaggacgtggtgaaggagctgaagaaggctctgtgcaatcctcacattcaagctgataggctgcgctaccggaatgtcatccagcgagtgattaggtacatgactcaaggcttggacatgtctggtgtttttatggaaatggtgaaggccagtgccactgtagatattgtccagaagaagttggtttatctgtacatgtgcacatatgctcccctgaaaccagatctggctctcctggccatcaatacgctgtgcaaagactgctcagaccccaatccaatggtgcgagggctggcgttacggagcatgtgtagcctcaggatgcctggtgtgcaggagtatatacaacagcctattctcaatggtctgcgggataaggcttcatatgtcaggagagtggcagtccttggatgtgccaagatgcataatcttcatggagactctgaagtagatggtgccctggtaaatgaattatacagtttgctgcgtgaccaggatccaattgtagttgtgaactgcttgaggtctctagaggaaattctgaaacaggaaggaggcgttgtcatcaataagcccattgctcaccatctcttaaatcgaatgtcaaaactggaccaatggggccaggctgaagtattgaactttctgctacgctaccaaccccgcagtgaggaagaactatttgacattctcaatctgttggatagtttcctcaagagcagtagcccaggtgtggtgatgggagctaccaaactttttctgatcttggcaaaaatgtttccccacgtacaaactgatgtccttgtgcgggtcaagggacctttgctagctgcctgttcttcagagagccgtgagctctgttttgttgctctttgtcatgtacgccagatcttgcatagtttaccaggtcactttagcagccactacaaaaagtttttttgctcctactcggagccccactacatcaaactacagaaagtggaggtgctgtgtgaactggtgaacgatgagaatgtgcagcaggtgctagaggagcttcgagggtactgcacggatgtgtctgcggactttgcacaggctgccatctttgccataggtggcattgccaggacttacacagatcaatgtgttcagattttaacagagttgctgggtcttcgacaagagcacattaccacagtggtggtgcagactttccgagacctggtttggttgtgtcctcagtgtactgaagctgtatgtcaggccctgcccggctgtgaagagaacattcaagatagtgaggggaagcaagcacttatttggctacttggtgtccatggggaaagaattcctaatgctccttatgtgttagaggactttgttgagaatgtgaagtcggaaacatttccagctgttaagatggagctgctcactgctttgctgcgccttttcctctcccgacctgctgagtgccaggacatgctaggacgtttgttgtattactgcatagaggaagaaaaagatatggctgtacgggaccgaggtctcttctattatcgcctcctcttagttggcattgatgaagttaagcggattctgtgtagccctaaatctgaccctactcttggacttttggaggatccggcagaaagacctgtgaatagctgggcctcagacttcaacacactggtgccagtgtatggcaaagcccactgggcaactatctctaaatgccagggggcagagcgttgtgacccagagcttcctaaaacttcatcctttgccgcatcaggacccttgattcctgaagagaacaaggagagggtacaagaactccctgattctggagccctcatgctagtccccaatcgccagcttactgctgattattttgagaaaacttggcttagccttaaagttgctcatcagcaagtgttgccttggcggggagaattccatcctgacaccctccagatggctcttcaagtagtgaacatccagaccatcgcaatgagtagggctgggtctcggccatggaaagcatacctcagtgctcaggatgatactggctgtctgttcttaacagaactgctattggagcctggaaactcagaaatgcagatctctgtgaaacaaaatgaagcaagaacggagacgctgaatagttttatttctgtattagaaactgtgattggaacaattgaagaaataaaatcataagcggccgcaaagcttgcctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 13 - DNA, 5813 bp, synthetic construct
  misc_feature 1..5813: note = pAAV-JeT-hAP4B1-Kan
  source 1..5813: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtccgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggaattccccggggatcctctagagtcgacgccaccatgccgtaccttggctccgaggacgtggtgaaggagctgaagaaggctctgtgcaatcctcacattcaagctgataggctgcgctaccggaatgtcatccagcgagtgattaggtacatgactcaaggcttggacatgtctggtgtttttatggaaatggtgaaggccagtgccactgtagatattgtccagaagaagttggtttatctgtacatgtgcacatatgctcccctgaaaccagatctggctctcctggccatcaatacgctgtgcaaagactgctcagaccccaatccaatggtgcgagggctggcgttacggagcatgtgtagcctcaggatgcctggtgtgcaggagtatatacaacagcctattctcaatggtctgcgggataaggcttcatatgtcaggagagtggcagtccttggatgtgccaagatgcataatcttcatggagactctgaagtagatggtgccctggtaaatgaattatacagtttgctgcgtgaccaggatccaattgtagttgtgaactgcttgaggtctctagaggaaattctgaaacaggaaggaggcgttgtcatcaataagcccattgctcaccatctcttaaatcgaatgtcaaaactggaccaatggggccaggctgaagtattgaactttctgctacgctaccaaccccgcagtgaggaagaactatttgacattctcaatctgttggatagtttcctcaagagcagtagcccaggtgtggtgatgggagctaccaaactttttctgatcttggcaaaaatgtttccccacgtacaaactgatgtccttgtgcgggtcaagggacctttgctagctgcctgttcttcagagagccgtgagctctgttttgttgctctttgtcatgtacgccagatcttgcatagtttaccaggtcactttagcagccactacaaaaagtttttttgctcctactcggagccccactacatcaaactacagaaagtggaggtgctgtgtgaactggtgaacgatgagaatgtgcagcaggtgctagaggagcttcgagggtactgcacggatgtgtctgcggactttgcacaggctgccatctttgccataggtggcattgccaggacttacacagatcaatgtgttcagattttaacagagttgctgggtcttcgacaagagcacattaccacagtggtggtgcagactttccgagacctggtttggttgtgtcctcagtgtactgaagctgtatgtcaggccctgcccggctgtgaagagaacattcaagatagtgaggggaagcaagcacttatttggctacttggtgtccatggggaaagaattcctaatgctccttatgtgttagaggactttgttgagaatgtgaagtcggaaacatttccagctgttaagatggagctgctcactgctttgctgcgccttttcctctcccgacctgctgagtgccaggacatgctaggacgtttgttgtattactgcatagaggaagaaaaagatatggctgtacgggaccgaggtctcttctattatcgcctcctcttagttggcattgatgaagttaagcggattctgtgtagccctaaatctgaccctactcttggacttttggaggatccggcagaaagacctgtgaatagctgggcctcagacttcaacacactggtgccagtgtatggcaaagcccactgggcaactatctctaaatgccagggggcagagcgttgtgacccagagcttcctaaaacttcatcctttgccgcatcaggacccttgattcctgaagagaacaaggagagggtacaagaactccctgattctggagccctcatgctagtccccaatcgccagcttactgctgattattttgagaaaacttggcttagccttaaagttgctcatcagcaagtgttgccttggcggggagaattccatcctgacaccctccagatggctcttcaagtagtgaacatccagaccatcgcaatgagtagggctgggtctcggccatggaaagcatacctcagtgctcaggatgatactggctgtctgttcttaacagaactgctattggagcctggaaactcagaaatgcagatctctgtgaaacaaaatgaagcaagaacggagacgctgaatagttttatttctgtattagaaactgtgattggaacaattgaagaaataaaatcataagcggccgcaaagcttgcctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 14 - DNA, 5874 bp, synthetic construct
  misc_feature 1..5874: note = pAAV-MeP229-hAP4B1-Kan
  source 1..5874: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtcccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaggaattccccggggatcctctagagtcgacgccaccatgccgtaccttggctccgaggacgtggtgaaggagctgaagaaggctctgtgcaatcctcacattcaagctgataggctgcgctaccggaatgtcatccagcgagtgattaggtacatgactcaaggcttggacatgtctggtgtttttatggaaatggtgaaggccagtgccactgtagatattgtccagaagaagttggtttatctgtacatgtgcacatatgctcccctgaaaccagatctggctctcctggccatcaatacgctgtgcaaagactgctcagaccccaatccaatggtgcgagggctggcgttacggagcatgtgtagcctcaggatgcctggtgtgcaggagtatatacaacagcctattctcaatggtctgcgggataaggcttcatatgtcaggagagtggcagtccttggatgtgccaagatgcataatcttcatggagactctgaagtagatggtgccctggtaaatgaattatacagtttgctgcgtgaccaggatccaattgtagttgtgaactgcttgaggtctctagaggaaattctgaaacaggaaggaggcgttgtcatcaataagcccattgctcaccatctcttaaatcgaatgtcaaaactggaccaatggggccaggctgaagtattgaactttctgctacgctaccaaccccgcagtgaggaagaactatttgacattctcaatctgttggatagtttcctcaagagcagtagcccaggtgtggtgatgggagctaccaaactttttctgatcttggcaaaaatgtttccccacgtacaaactgatgtccttgtgcgggtcaagggacctttgctagctgcctgttcttcagagagccgtgagctctgttttgttgctctttgtcatgtacgccagatcttgcatagtttaccaggtcactttagcagccactacaaaaagtttttttgctcctactcggagccccactacatcaaactacagaaagtggaggtgctgtgtgaactggtgaacgatgagaatgtgcagcaggtgctagaggagcttcgagggtactgcacggatgtgtctgcggactttgcacaggctgccatctttgccataggtggcattgccaggacttacacagatcaatgtgttcagattttaacagagttgctgggtcttcgacaagagcacattaccacagtggtggtgcagactttccgagacctggtttggttgtgtcctcagtgtactgaagctgtatgtcaggccctgcccggctgtgaagagaacattcaagatagtgaggggaagcaagcacttatttggctacttggtgtccatggggaaagaattcctaatgctccttatgtgttagaggactttgttgagaatgtgaagtcggaaacatttccagctgttaagatggagctgctcactgctttgctgcgccttttcctctcccgacctgctgagtgccaggacatgctaggacgtttgttgtattactgcatagaggaagaaaaagatatggctgtacgggaccgaggtctcttctattatcgcctcctcttagttggcattgatgaagttaagcggattctgtgtagccctaaatctgaccctactcttggacttttggaggatccggcagaaagacctgtgaatagctgggcctcagacttcaacacactggtgccagtgtatggcaaagcccactgggcaactatctctaaatgccagggggcagagcgttgtgacccagagcttcctaaaacttcatcctttgccgcatcaggacccttgattcctgaagagaacaaggagagggtacaagaactccctgattctggagccctcatgctagtccccaatcgccagcttactgctgattattttgagaaaacttggcttagccttaaagttgctcatcagcaagtgttgccttggcggggagaattccatcctgacaccctccagatggctcttcaagtagtgaacatccagaccatcgcaatgagtagggctgggtctcggccatggaaagcatacctcagtgctcaggatgatactggctgtctgttcttaacagaactgctattggagcctggaaactcagaaatgcagatctctgtgaaacaaaatgaagcaagaacggagacgctgaatagttttatttctgtattagaaactgtgattggaacaattgaagaaataaaatcataagcggccgcaaagcttgcctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 15 - DNA, 2220 bp, Homo sapiens
  source 1..2220: mol_type = other DNA
atgccgtaccttggctccgaggacgtggtgaaggagctgaagaaggctctgtgcaatcctcacattcaagctgataggctgcgctaccggaatgtcatccagcgagtgattaggtacatgactcaaggcttggacatgtctggtgtttttatggaaatggtgaaggccagtgccactgtagatattgtccagaagaagttggtttatctgtacatgtgcacatatgctcccctgaaaccagatctggctctcctggccatcaatacgctgtgcaaagactgctcagaccccaatccaatggtgcgagggctggcgttacggagcatgtgtagcctcaggatgcctggtgtgcaggagtatatacaacagcctattctcaatggtctgcgggataaggcttcatatgtcaggagagtggcagtccttggatgtgccaagatgcataatcttcatggagactctgaagtagatggtgccctggtaaatgaattatacagtttgctgcgtgaccaggatccaattgtagttgtgaactgcttgaggtctctagaggaaattctgaaacaggaaggaggcgttgtcatcaataagcccattgctcaccatctcttaaatcgaatgtcaaaactggaccaatggggccaggctgaagtattgaactttctgctacgctaccaaccccgcagtgaggaagaactatttgacattctcaatctgttggatagtttcctcaagagcagtagcccaggtgtggtgatgggagctaccaaactttttctgatcttggcaaaaatgtttccccacgtacaaactgatgtccttgtgcgggtcaagggacctttgctagctgcctgttcttcagagagccgtgagctctgttttgttgctctttgtcatgtacgccagatcttgcatagtttaccaggtcactttagcagccactacaaaaagtttttttgctcctactcggagccccactacatcaaactacagaaagtggaggtgctgtgtgaactggtgaacgatgagaatgtgcagcaggtgctagaggagcttcgagggtactgcacggatgtgtctgcggactttgcacaggctgccatctttgccataggtggcattgccaggacttacacagatcaatgtgttcagattttaacagagttgctgggtcttcgacaagagcacattaccacagtggtggtgcagactttccgagacctggtttggttgtgtcctcagtgtactgaagctgtatgtcaggccctgcccggctgtgaagagaacattcaagatagtgaggggaagcaagcacttatttggctacttggtgtccatggggaaagaattcctaatgctccttatgtgttagaggactttgttgagaatgtgaagtcggaaacatttccagctgttaagatggagctgctcactgctttgctgcgccttttcctctcccgacctgctgagtgccaggacatgctaggacgtttgttgtattactgcatagaggaagaaaaagatatggctgtacgggaccgaggtctcttctattatcgcctcctcttagttggcattgatgaagttaagcggattctgtgtagccctaaatctgaccctactcttggacttttggaggatccggcagaaagacctgtgaatagctgggcctcagacttcaacacactggtgccagtgtatggcaaagcccactgggcaactatctctaaatgccagggggcagagcgttgtgacccagagcttcctaaaacttcatcctttgccgcatcaggacccttgattcctgaagagaacaaggagagggtacaagaactccctgattctggagccctcatgctagtccccaatcgccagcttactgctgattattttgagaaaacttggcttagccttaaagttgctcatcagcaagtgttgccttggcggggagaattccatcctgacaccctccagatggctcttcaagtagtgaacatccagaccatcgcaatgagtagggctgggtctcggccatggaaagcatacctcagtgctcaggatgatactggctgtctgttcttaacagaactgctattggagcctggaaactcagaaatgcagatctctgtgaaacaaaatgaagcaagaacggagacgctgaatagttttatttctgtattagaaactgtgattggaacaattgaagaaataaaatcataa

SEQ ID NO: 16 - protein, 739 aa, Homo sapiens
MPYLGSEDVVKELKKALCNPHIQADRLRYRNVIQRVIRYMTQGLDMSGVFMEMVKASATVDIVQKKLVYLYMCTYAPLKPDLALLAINTLCKDCSDPNPMVRGLALRSMCSLRMPGVQEYIQQPILNGLRDKASYVRRVAVLGCAKMHNLHGDSEVDGALVNELYSLLRDQDPIVVVNCLRSLEEILKQEGGVVINKPIAHHLLNRMSKLDQWGQAEVLNFLLRYQPRSEEELFDILNLLDSFLKSSSPGVVMGATKLFLILAKMFPHVQTDVLVRVKGPLLAACSSESRELCFVALCHVRQILHSLPGHFSSHYKKFFCSYSEPHYIKLQKVEVLCELVNDENVQQVLEELRGYCTDVSADFAQAAIFAIGGIARTYTDQCVQILTELLGLRQEHITTVVVQTFRDLVWLCPQCTEAVCQALPGCEENIQDSEGKQALIWLLGVHGERIPNAPYVLEDFVENVKSETFPAVKMELLTALLRLFLSRPAECQDMLGRLLYYCIEEEKDMAVRDRGLFYYRLLLVGIDEVKRILCSPKSDPTLGLLEDPAERPVNSWASDFNTLVPVYGKAHWATISKCQGAERCDPELPKTSSFAASGPLIPEENKERVQELPDSGALMLVPNRQLTADYFEKTWLSLKVAHQQVLPWRGEFHPDTLQMALQVVNIQTIAMSRAGSRPWKAYLSAQDDTGCLFLTELLLEPGNSEMQISVKQNEARTETLNSFISVLETVIGTIEEIKS

SEQ ID NO: 17 - DNA, 3414 bp, Homo sapiens
  source 1..3414: mol_type = other DNA
atgagcgacatagtggagaagacgctgacggcgctgccgggactctttctgcagaaccagcccggtggtgggcccgcggccgccaaggcgtccttctcctcgaggctgggcagccttgtccgcggcatcacagccctcacctccaagcacgaagaagaaaaattaatccagcaggaactgagtagtctgaaagcgactgtttctgctcctactacaacactgaaaatgatgaaggaatgtatggtgagacttatatattgtgaaatgcttggatatgatgcttcctttggctatattcatgcaatcaagttagcccaacaaggaaacctcttagaaaaaagagtaggttatttggctgtttccttatttctacatgaaagtcatgaattattgcttctccttgtgaatacagttgtaaaggatctgcagagcactaacctagtagaagtgtgtatggcactgactgttgttagccagattttcccctgcgaaatgattccagctgttcttccattaatagaagataaacttcaacattctaaggagattgtacgaagaaaagctgttctggcattatacaaattccatctcattgctcctaatcaagtacaacatattcatattaagtttcggaaagcactttgtgacagagatgttggggtcatggctgcctccttgcatatatatcttagaatgattaaggagaattcatctggatataaagacttgactgggagttttgtaaccattttgaagcaagtagttggaggaaagctcccagtagaattcaattaccacagtgtgccagcaccatggttacaaattcagctcttgagaatactgggacttctaggaaaagatgatcaaaggacaagtgaattaatgtatgatgttcttgatgaatccttacgaagagctgagttaaatcacaatgtcacatatgctattttgtttgaatgtgtgcatacagtctattctatttatcctaaatcggaattacttgagaaggctgccaagtgcattggaaaatttgttctgtcacctaaaataaatctaaaatatttaggactgaaggctcttacctatgttatccaacaggatcctactctggctcttcaacaccagatgacaataattgaatgtttagatcatcctgatcccattattaaaagagagactctggaacttctttacagaattactaatgcacagaatataacagttattgtccagaaaatgcttgaatatttacatcagagcaaagaagagtatgtcatcgtcaatttggtcggcaaaatagcagagctggctgagaaatatgctcctgataatgcatggtttattcagacaatgaatgctgtgttttcagtaggaggagatgtaatgcatcctgatattcccaataactttctgagactactagcggaaggttttgatgatgaaacagaagatcagcaattaagactctatgcagttcagtcttatctcactttactggatatggaaaatgtgttctatccacagagatttcttcaagttatgagttgggtattaggggaatattcctacctcttagataaggaaacgccagaggaagttatagctaagctctacaagttacttatgaatgactctgtgtcttcagaaacaaaagcctggttaattgctgctgtgaccaaattgacatctcaggcgcactcttctaatacagttgagagattaatccatgaatttaccatatctttggatacttgtatgagacaacatgcatttgaattaaaacatttgcatgagaatgtggaacttatgaagagcttgcttccagttgacaggagttgtgaagacttggtggtagatgcttctttatcttttctggatggttttgtggctgaaggactcagtcagggtgcagcgccttacaaacctccccatcaacgccaggaggaaaagctttctcaggaaaaagttctcaattttgaaccatatggactctccttttcttcatctggcttcactggacgacagtctcctgctggcatttctcttggttcagatgtatctgggaatagtgctgagacaggactgaaagagacaaatagcttgaagctggaaggtataaagaaattgtgggggaaagaaggctatcttcccaagaaggaaagcaaaactggtgatgaaagtggagctctgcctgttcctcaagagagtataatggagaatgtagatcaagctataactaaaaaggatcaatctcaagttcttacccaatctaaagaggagaaagaaaagcagctgctggcatcatcattatttgttggtctaggatcagaaagtacaatcaacctgctgggaaaagcagatactgtctctcacaagttcagaaggaaatcaaaagtcaaagaagctaaaagtggcgaaacaaccagtactcataatatgacctgttcttcctttagttctttgtcaaatgtggcatatgaagatgattattattcgaatactttgcacgatacaggagacaaggaattaaagaaattttctctcacttcagaacttttggattctgagtcactcacagaactgcccttggttgagaaattctcatattgtagtctgtctacaccttcattgtttgctaataacaacatggaaatttttcaccctcctcaatctactgcagcctcagttgccaaggaaagctctttagcttcatcttttttggaagaaactactgaatacatacactcaaatgctatggaagtctgtaataatgaaactatatcagtgtcttcttataaaatttggaaagatgattgtttattgatggtctggtcagtcactaataagagtggtttggaattgaaaagtgctgacttagaaatttttcctgcagaaaatttcaaggtgactgagcaacctggatgctgtttgcctgtaatggaagcagaaagcaccaaaagctttcaatatagtgtgcagatagaaaaaccttttacagaaggaaatcttactggttttattagttatcatatgatggatactcattctgctcagctggaattttctgtaaacttatcactattagatttcattagaccattaaaaatctcaagtgacgactttgggaaactctggttatccttcgcaaatgatgtgaaacaaaatgtaaaaatgtcagaatctcaagctgcacttccttctgcactaaagactctgcaacagaaactaagactccatattattgagattataggcaatgaagggctattggcctgtcagctgctcccatccatcccctgcttactgcattgccgagttcatgcagatgtattagccctgtggttcagatcctcctgttctactcttcctgactatttactgtatcagtgtcaaaaggtgatggagggatcctag

SEQ ID NO: 18 - protein, 1137 aa, Homo sapiens
MSDIVEKTLTALPGLFLQNQPGGGPAAAKASFSSRLGSLVRGITALTSKHEEEKLIQQELSSLKATVSAPTTTLKMMKECMVRLIYCEMLGYDASFGYIHAIKLAQQGNLLEKRVGYLAVSLFLHESHELLLLLVNTVVKDLQSTNLVEVCMALTVVSQIFPCEMIPAVLPLIEDKLQHSKEIVRRKAVLALYKFHLIAPNQVQHIHIKFRKALCDRDVGVMAASLHIYLRMIKENSSGYKDLTGSFVTILKQVVGGKLPVEFNYHSVPAPWLQIQLLRILGLLGKDDQRTSELMYDVLDESLRRAELNHNVTYAILFECVHTVYSIYPKSELLEKAAKCIGKFVLSPKINLKYLGLKALTYVIQQDPTLALQHQMTIIECLDHPDPIIKRETLELLYRITNAQNITVIVQKMLEYLHQSKEEYVIVNLVGKIAELAEKYAPDNAWFIQTMNAVFSVGGDVMHPDIPNNFLRLLAEGFDDETEDQQLRLYAVQSYLTLLDMENVFYPQRFLQVMSWVLGEYSYLLDKETPEEVIAKLYKLLMNDSVSSETKAWLIAAVTKLTSQAHSSNTVERLIHEFTISLDTCMRQHAFELKHLHENVELMKSLLPVDRSCEDLVVDASLSFLDGFVAEGLSQGAAPYKPPHQRQEEKLSQEKVLNFEPYGLSFSSSGFTGRQSPAGISLGSDVSGNSAETGLKETNSLKLEGIKKLWGKEGYLPKKESKTGDESGALPVPQESIMENVDQAITKKDQSQVLTQSKEEKEKQLLASSLFVGLGSESTINLLGKADTVSHKFRRKSKVKEAKSGETTSTHNMTCSSFSSLSNVAYEDDYYSNTLHDTGDKELKKFSLTSELLDSESLTELPLVEKFSYCSLSTPSLFANNNMEIFHPPQSTAASVAKESSLASSFLEETTEYIHSNAMEVCNNETISVSSYKIWKDDCLLMVWSVTNKSGLELKSADLEIFPAENFKVTEQPGCCLPVMEAESTKSFQYSVQIEKPFTEGNLTGFISYHMMDTHSAQLEFSVNLSLLDFIRPLKISSDDFGKLWLSFANDVKQNVKMSESQAALPSALKTLQQKLRLHIIEIIGNEGLLACQLLPSIPCLLHCRVHADVLALWFRSSCSTLPDYLLYQCQKVMEGS

SEQ ID NO: 19 - DNA, 1362 bp, Homo sapiens
  source 1..1362: mol_type = other DNA
atgatttcccaattcttcattctgtcctccaagggggacccgctcatctacaaagacttccgcggggacagtggcggccgggatgtggccgagctcttctaccggaagctgacgggactgccaggagacgagtccccggttgtcatgcatcaccatggccgtcatttcattcacatcagacacagcggcctctatttggtggtcacaacttcagaaaacgtttctcccttcagcctcctagaactgctctccaggttggccacccttctgggcgattactgtggctccctgggcgaggggaccatctcccgcaatgtggctctggtatacgaactcctggatgaagtgctggactatggctatgtacagaccacatccacggagatgctgaggaatttcatccagacggaagctgtggtcagcaagcccttcagcctctttgacctcagcagcgttggcttgtttggggctgagacacaacagagcaaagtggcccccagcagtgcagccagccgccccgtcctgtccagtcgctctgaccagagccaaaagaatgaagtttttttggatgtggtcgagagattgtctgtactgatagcatctaatggatccctgctgaaggtggatgtgcagggagagattcggctcaagagcttccttcctagcggctctgagatgcgcattggcttgacggaagagttttgtgtggggaagtcagagctgagaggttatgggccaggaatccgggtcgatgaagtctcgtttcacagctctgtgaatctggacgaatttgagtctcatcgaatcctccgcttgcaaccacctcagggcgagctgactgtgatgcggtaccaactctccgatgacctcccctcaccgctccccttccggctcttcccctctgtgcagtgggaccgaggctcaggccggctccaggtttatctaaagttgcgatgtgacctgctctcaaagagccaagccctcaatgtcaggctgcacctccccctgcctcgaggggtggtcagcctgtctcaggagctgagcagcccagagcagaaggctgagctggcagagggagcccttcgctgggacctgcctcgggtgcaaggaggctctcaactctcaggccttttccagatggacgtcccagggcccccaggacctcccagccatgggctctccacctcggcctctcctctggggctgggccctgccagtctctccttcgagcttccccggcacacgtgctctggcctccaggtccgattcctcaggctggccttcaggccatgcggcaatgccaacccccacaagtgggtgcgacacctaagccacagcgacgcctatgtcattcggatctga

SEQ ID NO: 20 - protein, 453 aa, Homo sapiens
MISQFFILSSKGDPLIYKDFRGDSGGRDVAELFYRKLTGLPGDESPVVMHHHGRHFIHIRHSGLYLVVTTSENVSPFSLLELLSRLATLLGDYCGSLGEGTISRNVALVYELLDEVLDYGYVQTTSTEMLRNFIQTEAVVSKPFSLFDLSSVGLFGAETQQSKVAPSSAASRPVLSSRSDQSQKNEVFLDVVERLSVLIASNGSLLKVDVQGEIRLKSFLPSGSEMRIGLTEEFCVGKSELRGYGPGIRVDEVSFHSSVNLDEFESHRILRLQPPQGELTVMRYQLSDDLPSPLPFRLFPSVQWDRGSGRLQVYLKLRCDLLSKSQALNVRLHLPLPRGVVSLSQELSSPEQKAELAEGALRWDLPRVQGGSQLSGLFQMDVPGPPGPPSHGLSTSASPLGLGPASLSFELPRHTCSGLQVRFLRLAFRPCGNANPHKWVRHLSHSDAYVIRI

SEQ ID NO: 21 - DNA, 480 bp, Homo sapiens
  source 1..480: mol_type = other DNA
atgataaaatttttcctcatggtgaataaacaagggcagactcgactttctaagtactatgaacatgtggatattaataagcgtacacttctggaaacagaagtcataaagagctgtctctctcgatccaatgaacaatgctctttcattgaatataaggattttaagctgatatatcggcagtatgcagctctcttcattgtggttggagttaatgacactgagaacgagatggctatttatgaattcattcataactttgtggaagttttagatgagtatttcagccgagtgagtgaattagatgtatcctttttcaatactgttttccacagtacttggcaaatgcactctggtccttatcaggaaccaattgatgaacttcccaaaatatgctcagccctggagccccagcagacttgcttttctccagacagttcatcattcaaaggagctgcctccaccacccccatctactga

SEQ ID NO: 22 - protein, 159 aa, Homo sapiens
MIKFFLMVNKQGQTRLSKYYEHVDINKRTLLETEVIKSCLSRSNEQCSFIEYKDFKLIYRQYAALFIVVGVNDTENEMAIYEFIHNFVEVLDEYFSRVSELDVSFFNTVFHSTWQMHSGPYQEPIDELPKICSALEPQQTCFSPDSSSFKGAASTTPIY

SEQ ID NO: 23 - DNA, 6494 bp, synthetic construct
  misc_feature 1..6494: note = vector sequence
  source 1..6494: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtcgaattccccggggatcctctagagtcgacgccaccatgccgtaccttggctccgaggacgtggtgaaggagctgaagaaggctctgtgcaatcctcacattcaagctgataggctgcgctaccggaatgtcatccagcgagtgattaggtacatgactcaaggcttggacatgtctggtgtttttatggaaatggtgaaggccagtgccactgtagatattgtccagaagaagttggtttatctgtacatgtgcacatatgctcccctgaaaccagatctggctctcctggccatcaatacgctgtgcaaagactgctcagaccccaatccaatggtgcgagggctggcgttacggagcatgtgtagcctcaggatgcctggtgtgcaggagtatatacaacagcctattctcaatggtctgcgggataaggcttcatatgtcaggagagtggcagtccttggatgtgccaagatgcataatcttcatggagactctgaagtagatggtgccctggtaaatgaattatacagtttgctgcgtgaccaggatccaattgtagttgtgaactgcttgaggtctctagaggaaattctgaaacaggaaggaggcgttgtcatcaataagcccattgctcaccatctcttaaatcgaatgtcaaaactggaccaatggggccaggctgaagtattgaactttctgctacgctaccaaccccgcagtgaggaagaactatttgacattctcaatctgttggatagtttcctcaagagcagtagcccaggtgtggtgatgggagctaccaaactttttctgatcttggcaaaaatgtttccccacgtacaaactgatgtccttgtgcgggtcaagggacctttgctagctgcctgttcttcagagagccgtgagctctgttttgttgctctttgtcatgtacgccagatcttgcatagtttaccaggtcactttagcagccactacaaaaagtttttttgctcctactcggagccccactacatcaaactacagaaagtggaggtgctgtgtgaactggtgaacgatgagaatgtgcagcaggtgctagaggagcttcgagggtactgcacggatgtgtctgcggactttgcacaggctgccatctttgccataggtggcattgccaggacttacacagatcaatgtgttcagattttaacagagttgctgggtcttcgacaagagcacattaccacagtggtggtgcagactttccgagacctggtttggttgtgtcctcagtgtactgaagctgtatgtcaggccctgcccggctgtgaagagaacattcaagatagtgaggggaagcaagcacttatttggctacttggtgtccatggggaaagaattcctaatgctccttatgtgttagaggactttgttgagaatgtgaagtcggaaacatttccagctgttaagatggagctgctcactgctttgctgcgccttttcctctcccgacctgctgagtgccaggacatgctaggacgtttgttgtattactgcatagaggaagaaaaagatatggctgtacgggaccgaggtctcttctattatcgcctcctcttagttggcattgatgaagttaagcggattctgtgtagccctaaatctgaccctactcttggacttttggaggatccggcagaaagacctgtgaatagctgggcctcagacttcaacacactggtgccagtgtatggcaaagcccactgggcaactatctctaaatgccagggggcagagcgttgtgacccagagcttcctaaaacttcatcctttgccgcatcaggacccttgattcctgaagagaacaaggagagggtacaagaactccctgattctggagccctcatgctagtccccaatcgccagcttactgctgattattttgagaaaacttggcttagccttaaagttgctcatcagcaagtgttgccttggcggggagaattccatcctgacaccctccagatggctcttcaagtagtgaacatccagaccatcgcaatgagtagggctgggtctcggccatggaaagcatacctcagtgctcaggatgatactggctgtctgttcttaacagaactgctattggagcctggaaactcagaaatgcagatctctgtgaaacaaaatgaagcaagaacggagacgctgaatagttttatttctgtattagaaactgtgattggaacaattgaagaaataaaatcataagcggccgcaagcttgcctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaatttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 24 - DNA, 6148 bp, synthetic construct
  misc_feature 1..6148: note = vector sequence
  source 1..6148: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtccagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggttggaccggtcgaattccccggggatcctctagagtcgacgccaccatgccgtaccttggctccgaggacgtggtgaaggagctgaagaaggctctgtgcaatcctcacattcaagctgataggctgcgctaccggaatgtcatccagcgagtgattaggtacatgactcaaggcttggacatgtctggtgtttttatggaaatggtgaaggccagtgccactgtagatattgtccagaagaagttggtttatctgtacatgtgcacatatgctcccctgaaaccagatctggctctcctggccatcaatacgctgtgcaaagactgctcagaccccaatccaatggtgcgagggctggcgttacggagcatgtgtagcctcaggatgcctggtgtgcaggagtatatacaacagcctattctcaatggtctgcgggataaggcttcatatgtcaggagagtggcagtccttggatgtgccaagatgcataatcttcatggagactctgaagtagatggtgccctggtaaatgaattatacagtttgctgcgtgaccaggatccaattgtagttgtgaactgcttgaggtctctagaggaaattctgaaacaggaaggaggcgttgtcatcaataagcccattgctcaccatctcttaaatcgaatgtcaaaactggaccaatggggccaggctgaagtattgaactttctgctacgctaccaaccccgcagtgaggaagaactatttgacattctcaatctgttggatagtttcctcaagagcagtagcccaggtgtggtgatgggagctaccaaactttttctgatcttggcaaaaatgtttccccacgtacaaactgatgtccttgtgcgggtcaagggacctttgctagctgcctgttcttcagagagccgtgagctctgttttgttgctctttgtcatgtacgccagatcttgcatagtttaccaggtcactttagcagccactacaaaaagtttttttgctcctactcggagccccactacatcaaactacagaaagtggaggtgctgtgtgaactggtgaacgatgagaatgtgcagcaggtgctagaggagcttcgagggtactgcacggatgtgtctgcggactttgcacaggctgccatctttgccataggtggcattgccaggacttacacagatcaatgtgttcagattttaacagagttgctgggtcttcgacaagagcacattaccacagtggtggtgcagactttccgagacctggtttggttgtgtcctcagtgtactgaagctgtatgtcaggccctgcccggctgtgaagagaacattcaagatagtgaggggaagcaagcacttatttggctacttggtgtccatggggaaagaattcctaatgctccttatgtgttagaggactttgttgagaatgtgaagtcggaaacatttccagctgttaagatggagctgctcactgctttgctgcgccttttcctctcccgacctgctgagtgccaggacatgctaggacgtttgttgtattactgcatagaggaagaaaaagatatggctgtacgggaccgaggtctcttctattatcgcctcctcttagttggcattgatgaagttaagcggattctgtgtagccctaaatctgaccctactcttggacttttggaggatccggcagaaagacctgtgaatagctgggcctcagacttcaacacactggtgccagtgtatggcaaagcccactgggcaactatctctaaatgccagggggcagagcgttgtgacccagagcttcctaaaacttcatcctttgccgcatcaggacccttgattcctgaagagaacaaggagagggtacaagaactccctgattctggagccctcatgctagtccccaatcgccagcttactgctgattattttgagaaaacttggcttagccttaaagttgctcatcagcaagtgttgccttggcggggagaattccatcctgacaccctccagatggctcttcaagtagtgaacatccagaccatcgcaatgagtagggctgggtctcggccatggaaagcatacctcagtgctcaggatgatactggctgtctgttcttaacagaactgctattggagcctggaaactcagaaatgcagatctctgtgaaacaaaatgaagcaagaacggagacgctgaatagttttatttctgtattagaaactgtgattggaacaattgaagaaataaaatcataagcggccgcaagcttgcctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaatttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 25 - DNA, 6300 bp, synthetic construct
  misc_feature 1..6300: note = vector sequence
  source 1..6300: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtccggcttccacaacggcgtcccgataaaagtcatgctggggcaagaacagggccagagaccctaagggaaagagcccagcagagcaataagttaccagagtgggcagaaaaaggaagtcaccctgcccgctggagcccaaatcgcggagggcgctggacactcccaaccgcgcgggagcggaggccgagccgggcactcaaagcgcgcgctgcagggaggagacaggccaaagtagccgatttccgattgcccgccgcagctccaccagagggagccgcgcagctgcgcctggagagtgagaggttgggggaagggcatcctaaggtcggagtcgggactacgcttagcgctcgctcgcggggagaccggcagcggtcttctgtttggtctcttgacaagggggcgggacttccgcagaaagcaagattgggcctatccgcgatgagaacggcgtggagggggaattaaggctggaactggagagggaggggtctgcggtcagtcggacgcggcgctggaaatccggcggaggacctagaagtagcgggagcgatctgtgggcggggcgaacggcctgccggccgacggtagggcctgaagagttggaccggtcgaattccccggggatcctctagagtcgacgccaccatgccgtaccttggctccgaggacgtggtgaaggagctgaagaaggctctgtgcaatcctcacattcaagctgataggctgcgctaccggaatgtcatccagcgagtgattaggtacatgactcaaggcttggacatgtctggtgtttttatggaaatggtgaaggccagtgccactgtagatattgtccagaagaagttggtttatctgtacatgtgcacatatgctcccctgaaaccagatctggctctcctggccatcaatacgctgtgcaaagactgctcagaccccaatccaatggtgcgagggctggcgttacggagcatgtgtagcctcaggatgcctggtgtgcaggagtatatacaacagcctattctcaatggtctgcgggataaggcttcatatgtcaggagagtggcagtccttggatgtgccaagatgcataatcttcatggagactctgaagtagatggtgccctggtaaatgaattatacagtttgctgcgtgaccaggatccaattgtagttgtgaactgcttgaggtctctagaggaaattctgaaacaggaaggaggcgttgtcatcaataagcccattgctcaccatctcttaaatcgaatgtcaaaactggaccaatggggccaggctgaagtattgaactttctgctacgctaccaaccccgcagtgaggaagaactatttgacattctcaatctgttggatagtttcctcaagagcagtagcccaggtgtggtgatgggagctaccaaactttttctgatcttggcaaaaatgtttccccacgtacaaactgatgtccttgtgcgggtcaagggacctttgctagctgcctgttcttcagagagccgtgagctctgttttgttgctctttgtcatgtacgccagatcttgcatagtttaccaggtcactttagcagccactacaaaaagtttttttgctcctactcggagccccactacatcaaactacagaaagtggaggtgctgtgtgaactggtgaacgatgagaatgtgcagcaggtgctagaggagcttcgagggtactgcacggatgtgtctgcggactttgcacaggctgccatctttgccataggtggcattgccaggacttacacagatcaatgtgttcagattttaacagagttgctgggtcttcgacaagagcacattaccacagtggtggtgcagactttccgagacctggtttggttgtgtcctcagtgtactgaagctgtatgtcaggccctgcccggctgtgaagagaacattcaagatagtgaggggaagcaagcacttatttggctacttggtgtccatggggaaagaattcctaatgctccttatgtgttagaggactttgttgagaatgtgaagtcggaaacatttccagctgttaagatggagctgctcactgctttgctgcgccttttcctctcccgacctgctgagtgccaggacatgctaggacgtttgttgtattactgcatagaggaagaaaaagatatggctgtacgggaccgaggtctcttctattatcgcctcctcttagttggcattgatgaagttaagcggattctgtgtagccctaaatctgaccctactcttggacttttggaggatccggcagaaagacctgtgaatagctgggcctcagacttcaacacactggtgccagtgtatggcaaagcccactgggcaactatctctaaatgccagggggcagagcgttgtgacccagagcttcctaaaacttcatcctttgccgcatcaggacccttgattcctgaagagaacaaggagagggtacaagaactccctgattctggagccctcatgctagtccccaatcgccagcttactgctgattattttgagaaaacttggcttagccttaaagttgctcatcagcaagtgttgccttggcggggagaattccatcctgacaccctccagatggctcttcaagtagtgaacatccagaccatcgcaatgagtagggctgggtctcggccatggaaagcatacctcagtgctcaggatgatactggctgtctgttcttaacagaactgctattggagcctggaaactcagaaatgcagatctctgtgaaacaaaatgaagcaagaacggagacgctgaatagttttatttctgtattagaaactgtgattggaacaattgaagaaataaaatcataagcggccgcaagcttgcctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaatttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 26 - DNA, 5864 bp, synthetic construct
  misc_feature 1..5864: note = vector sequence
  source 1..5864: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtccgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtgttggaccggtcgaattccccggggatcctctagagtcgacgccaccatgccgtaccttggctccgaggacgtggtgaaggagctgaagaaggctctgtgcaatcctcacattcaagctgataggctgcgctaccggaatgtcatccagcgagtgattaggtacatgactcaaggcttggacatgtctggtgtttttatggaaatggtgaaggccagtgccactgtagatattgtccagaagaagttggtttatctgtacatgtgcacatatgctcccctgaaaccagatctggctctcctggccatcaatacgctgtgcaaagactgctcagaccccaatccaatggtgcgagggctggcgttacggagcatgtgtagcctcaggatgcctggtgtgcaggagtatatacaacagcctattctcaatggtctgcgggataaggcttcatatgtcaggagagtggcagtccttggatgtgccaagatgcataatcttcatggagactctgaagtagatggtgccctggtaaatgaattatacagtttgctgcgtgaccaggatccaattgtagttgtgaactgcttgaggtctctagaggaaattctgaaacaggaaggaggcgttgtcatcaataagcccattgctcaccatctcttaaatcgaatgtcaaaactggaccaatggggccaggctgaagtattgaactttctgctacgctaccaaccccgcagtgaggaagaactatttgacattctcaatctgttggatagtttcctcaagagcagtagcccaggtgtggtgatgggagctaccaaactttttctgatcttggcaaaaatgtttccccacgtacaaactgatgtccttgtgcgggtcaagggacctttgctagctgcctgttcttcagagagccgtgagctctgttttgttgctctttgtcatgtacgccagatcttgcatagtttaccaggtcactttagcagccactacaaaaagtttttttgctcctactcggagccccactacatcaaactacagaaagtggaggtgctgtgtgaactggtgaacgatgagaatgtgcagcaggtgctagaggagcttcgagggtactgcacggatgtgtctgcggactttgcacaggctgccatctttgccataggtggcattgccaggacttacacagatcaatgtgttcagattttaacagagttgctgggtcttcgacaagagcacattaccacagtggtggtgcagactttccgagacctggtttggttgtgtcctcagtgtactgaagctgtatgtcaggccctgcccggctgtgaagagaacattcaagatagtgaggggaagcaagcacttatttggctacttggtgtccatggggaaagaattcctaatgctccttatgtgttagaggactttgttgagaatgtgaagtcggaaacatttccagctgttaagatggagctgctcactgctttgctgcgccttttcctctcccgacctgctgagtgccaggacatgctaggacgtttgttgtattactgcatagaggaagaaaaagatatggctgtacgggaccgaggtctcttctattatcgcctcctcttagttggcattgatgaagttaagcggattctgtgtagccctaaatctgaccctactcttggacttttggaggatccggcagaaagacctgtgaatagctgggcctcagacttcaacacactggtgccagtgtatggcaaagcccactgggcaactatctctaaatgccagggggcagagcgttgtgacccagagcttcctaaaacttcatcctttgccgcatcaggacccttgattcctgaagagaacaaggagagggtacaagaactccctgattctggagccctcatgctagtccccaatcgccagcttactgctgattattttgagaaaacttggcttagccttaaagttgctcatcagcaagtgttgccttggcggggagaattccatcctgacaccctccagatggctcttcaagtagtgaacatccagaccatcgcaatgagtagggctgggtctcggccatggaaagcatacctcagtgctcaggatgatactggctgtctgttcttaacagaactgctattggagcctggaaactcagaaatgcagatctctgtgaaacaaaatgaagcaagaacggagacgctgaatagttttatttctgtattagaaactgtgattggaacaattgaagaaataaaatcataagcggccgcaagcttgcctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaatttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 27 - DNA, 5925 bp, synthetic construct
  misc_feature 1..5925: note = vector sequence
  source 1..5925: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtcccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaagttggaccggtcgaattccccggggatcctctagagtcgacgccaccatgccgtaccttggctccgaggacgtggtgaaggagctgaagaaggctctgtgcaatcctcacattcaagctgataggctgcgctaccggaatgtcatccagcgagtgattaggtacatgactcaaggcttggacatgtctggtgtttttatggaaatggtgaaggccagtgccactgtagatattgtccagaagaagttggtttatctgtacatgtgcacatatgctcccctgaaaccagatctggctctcctggccatcaatacgctgtgcaaagactgctcagaccccaatccaatggtgcgagggctggcgttacggagcatgtgtagcctcaggatgcctggtgtgcaggagtatatacaacagcctattctcaatggtctgcgggataaggcttcatatgtcaggagagtggcagtccttggatgtgccaagatgcataatcttcatggagactctgaagtagatggtgccctggtaaatgaattatacagtttgctgcgtgaccaggatccaattgtagttgtgaactgcttgaggtctctagaggaaattctgaaacaggaaggaggcgttgtcatcaataagcccattgctcaccatctcttaaatcgaatgtcaaaactggaccaatggggccaggctgaagtattgaactttctgctacgctaccaaccccgcagtgaggaagaactatttgacattctcaatctgttggatagtttcctcaagagcagtagcccaggtgtggtgatgggagctaccaaactttttctgatcttggcaaaaatgtttccccacgtacaaactgatgtccttgtgcgggtcaagggacctttgctagctgcctgttcttcagagagccgtgagctctgttttgttgctctttgtcatgtacgccagatcttgcatagtttaccaggtcactttagcagccactacaaaaagtttttttgctcctactcggagccccactacatcaaactacagaaagtggaggtgctgtgtgaactggtgaacgatgagaatgtgcagcaggtgctagaggagcttcgagggtactgcacggatgtgtctgcggactttgcacaggctgccatctttgccataggtggcattgccaggacttacacagatcaatgtgttcagattttaacagagttgctgggtcttcgacaagagcacattaccacagtggtggtgcagactttccgagacctggtttggttgtgtcctcagtgtactgaagctgtatgtcaggccctgcccggctgtgaagagaacattcaagatagtgaggggaagcaagcacttatttggctacttggtgtccatggggaaagaattcctaatgctccttatgtgttagaggactttgttgagaatgtgaagtcggaaacatttccagctgttaagatggagctgctcactgctttgctgcgccttttcctctcccgacctgctgagtgccaggacatgctaggacgtttgttgtattactgcatagaggaagaaaaagatatggctgtacgggaccgaggtctcttctattatcgcctcctcttagttggcattgatgaagttaagcggattctgtgtagccctaaatctgaccctactcttggacttttggaggatccggcagaaagacctgtgaatagctgggcctcagacttcaacacactggtgccagtgtatggcaaagcccactgggcaactatctctaaatgccagggggcagagcgttgtgacccagagcttcctaaaacttcatcctttgccgcatcaggacccttgattcctgaagagaacaaggagagggtacaagaactccctgattctggagccctcatgctagtccccaatcgccagcttactgctgattattttgagaaaacttggcttagccttaaagttgctcatcagcaagtgttgccttggcggggagaattccatcctgacaccctccagatggctcttcaagtagtgaacatccagaccatcgcaatgagtagggctgggtctcggccatggaaagcatacctcagtgctcaggatgatactggctgtctgttcttaacagaactgctattggagcctggaaactcagaaatgcagatctctgtgaaacaaaatgaagcaagaacggagacgctgaatagttttatttctgtattagaaactgtgattggaacaattgaagaaataaaatcataagcggccgcaagcttgcctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaatttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 28 - DNA, 794 bp, synthetic construct
  misc_feature 1..794: note = promoter sequence
  source 1..794: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcag

SEQ ID NO: 29 - DNA, 19 bp, synthetic construct
  source 1..19: mol_type = other DNA
tcgcccgaggacccaagaa

SEQ ID NO: 30 - DNA, 27 bp, synthetic construct
  source 1..27: mol_type = other DNA
cctatcagcctgaatatgagggttaca

SEQ ID NO: 31 - DNA, 23 bp, synthetic construct
  source 1..23: mol_type = other DNA
gctggatgacattccggtatatg

SEQ ID NO: 32 - DNA, 20 bp, synthetic construct
  source 1..20: mol_type = other DNA
ctggtgaacgatgagaatgt

SEQ ID NO: 33 - DNA, 20 bp, synthetic construct
  source 1..20: mol_type = other DNA
gacccagcaactctgttaaa

SEQ ID NO: 34 - DNA, 20 bp, synthetic construct
  source 1..20: mol_type = other DNA
ctgtgctaggctcccacatc

SEQ ID NO: 35 - DNA, 20 bp, synthetic construct
  source 1..20: mol_type = other DNA
tggcactggcctttaccatt

SEQ ID NO: 36 - DNA, 19 bp, synthetic construct
  source 1..19: mol_type = other DNA
gtaacccgttgaaccccat

SEQ ID NO: 37 - DNA, 20 bp, synthetic construct
  source 1..20: mol_type = other DNA
ccatccaatcggtagtagcg

SEQ ID NO: 38 - DNA, 144 bp, synthetic construct
  source 1..144: mol_type = other DNA
ttggcgacgatgccataccttggctctgaggacgtggtgaaggaactgaagaaggctctgtgtaaccctcatattcaggctgataggctgcgctaccggaatgtcatccagcgagttattaggtatcaccaacctaccatagaa